(19) United States
(12) Patent Application Publication
(10) Pub. No.: US 2025/0302968 A1
(43) Pub. Date: Oct. 2, 2025

(54) RADIATION CLEAVED DRUG-CONJUGATE LINKERS ENABLE LOCAL PAYLOAD RELEASE SCLEROSIS

(71) Applicant: The General Hospital Corporation, Boston, MA (US)

(72) Inventors: Miles A. Miller, Sudbury, MA (US); Ralph Weissleder, Peabody, MA (US); Jeremy Quintana, Methuen, MA (US)

(21) Appl. No.: 18/864,320

(22) PCT Filed: May 11, 2023
(86) PCT No.: PCT/US2023/021896

§ 371 Date: Nov. 8, 2024

Related U.S. Application Data
(60) Provisional application No. 63/340,970, filed on May 12, 2022.

(51) Int. Cl. A61K 47/54; A61K 41/00; A61P 35/00

(52) U.S. Cl. CPC A61K 47/54; A61K 41/00; A61P 35/00; A61K 2121/00

(57) ABSTRACT

The present disclosure relates to drug-conjugate molecules that release a biologically active payload upon exposure to ionizing radiation. Localized x-ray irradiation releases the payload under normoxic and/or hypoxic conditions that are traditionally associated with radiotherapy resistance.

### CLAIM OF PRIORITY

This application claims the benefit of U.S. Patent Application Ser. No. 63/340,970, filed on May 12, 2022. The entire contents of the foregoing are hereby incorporated by reference.

### TECHNICAL FIELD

The present disclosure relates to drug conjugates to deliver biologically active payloads upon application of ionizing radiation to the conjugate.

### BACKGROUND

Numerous approaches have been developed to improve the therapeutic index of potent small molecule compounds by linking them as inactive prodrugs to biologics or nanoparticles (Liu, H.; Qian, F. Theranostics 2022, 12, 1321-1332; Liu, L.; Kshirsagar, P. G., et al. Theranostics 2022, 12, 1030-1060). For the treatment of metastatic cancers, cytotoxic agents have been bound to serum albumin (Hoogenboezem, E. N.; Duvall, C. L. Adv. Drug Delivery Rev. 2018, 130, 73-89) and nanoparticles (Mitchell, M. J. et al Nat. Rev. Drug Discovery 2021, 20, 101-124) to improve systemic pharmacokinetics and, in principle, to promote tumor accumulation via molecular targeting and/or “enhanced permeability and retention” (EPR) mechanisms of uptake. These mechanisms include oncogene-driven micropinocytosis (Li, R. et al. Nat. Nanotechnol. 2021, 16, 830-839; Commisso, C. et al. Nature 2013, 497, 633-637), permeable tumor vasculature (Inoue, Y. et al. J. Controlled Release 2021, 329, 63-75; Miller, M. A. et al. Sci. Transl. Med. 2017, 9, eaa10225), dysfunctional tumor lymphatics, and other features that contribute to what is collectively referred to as the EPR effect (Fang, J.; Islam, W.; Maeda, H. Adv. Drug Delivery Rev. 2020, 157, 142-160; Shi, Y. et al. Theranostics 2020, 10, 7921-7924; and Nia, H. et al. Science 2020, 370, aaz0868). Alternatively, molecularly-targeted strategies based on antibodies (Chau, C. H. et al. Lancet 2019, 394, 793-804; Beck, A. et al. Nat. Rev. Drug Discovery 2017, 16, 315-337) or functionalized nanoparticles (Liu, L et al. Theranostics 2022, 12, 1030-1060; Bertrand, N. et al. Adv. Drug Delivery Rev. 2014, 66, 2-25; and Fu, Z.; Xiang, J. Int. J. Mol. Sci. 2020, 21, 9123) have been designed to bind surface receptors selectively over-expressed by cancer cells. Multiple antibody drug conjugates (ADCs) and therapeutic nanoparticles (NPs) have received FDA-approval for clinical use. Yet despite their successes, these agents still accumulate in off-target tissues and elicit systemic toxicities (Birrer, M. J. et al. J. Natl. Cancer Inst. 2019, 111, 538-549; Drago, J. Z. et al. Nat. Rev. Clin. Oncol. 2021, 18, 327-344; and Joubert, N. et al. Pharmaceuticals 2020, 13, 245). Drugs receive black box warnings when they exhibit potentially serious and deadly adverse effects, and toxicities affecting the bone marrow, liver, and other organs have led to boxed warnings on the FDA package inserts of nearly all drug-conjugates in oncology.

One strategy to minimize off-target payload activity is to optimize the delivery vehicle by conjugating the drug payload to a biologic or nanoparticle. Vehicle accumulation in clearance organs and the mononuclear phagocyte system can be minimized through PEGylation or FcRn engineering, for instance (Tedeschini, T. et al. J. Controlled Release 2021, 337, 431-447; Lu, S. et al. ACS Appl. Mater. Interfaces 2021, 13, 46291-46302), but is nonetheless difficult to completely eliminate (Blanco, E. et al. Nat. Biotechnol. 2015, 33, 941-951). Choosing appropriate tumor-specific molecular targets can improve selective accumulation, but one would need to first identify a tumor-specific target and often only a subset of tumors or tumor cells may preferentially express such targets (Wang, Y.; Giaccone, G. Frontiers in Oncology 2011, 1, 4; Kinneer, K. et al. Clin. Cancer Res. 2018, 24, 6570-6582). Optimizing the chemistry by which a drug payload attaches to its delivery vehicle represents another set of strategies (Su, D. et al. Bioconjugate Chem. 2018, 29, 1155-1167; Pillow, T. H. et al. Chem. Sci. 2017, 8, 366-370; and Cilliers, C. et al. Cancer Res. 2018, 78, 758-768). Premature or off-target payload-release contributes to non-specific and/or systemic exposure (Shen, B.-Q. et al. Nat. Biotechnol. 2012, 30, 184-189), while non-cleavable linkers may insufficiently yield fully active payloads in tumors (Lambert, J. M.; Berkenblit, A. Annu. Rev. Med. 2018, 69, 191-207). Unfortunately, most current strategies for controlling payload release rely on pH, lysosomal degradation, protease activity and other processes that are not reliably unique to tumor cells (Mckertish, C. M.; Kayser, V. Biomedicines 2021, 9, 872).

Bio-orthogonal approaches aim to overcome limitations of biological specificity by modulating drug activity in a manner that is independent of naturally occurring chemical processes. Previously studied strategies for triggered activation of cancer prodrugs have used electrochemical (Norman, D. J. et al. J. Chem. Soc., Chem, Commun. 2018, 54, 9242-9245), ultrasonic (Bezagu, M. et al. Eur. J. Med. Chem. 2017, 142, 2-7), optical (Lerch, M. M. et al. Angew. Chem. Int. Ed. 2016, 55, 10978-10999; Zang, C. et al. Chem. Sci. 2019, 10, 8973-8980), copper-free click chemical (Wang, Y. et al. Molecules 2020, 25, 5640; Ji, X. et al. Chem. Soc. Rev. 2019, 48, 177-194), and transition-metal catalytic (Weiss, J. T. et al. Nat. Commun. 2014, 5, 3277; Miller, M. A. et al. ACS nano 2018, 12, 12814-12826) activation schemes.

### SUMMARY

Some embodiments provide compound of Formula (I), or a pharmaceutically acceptable salt thereof,

[RSM]-Linker-Drug Moiety (I)
wherein RSM is a radiation-sensitive moiety.

Some embodiments provide a compound of Formula (I-A), or a pharmaceutically acceptable salt thereof,

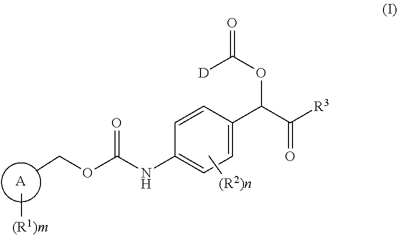

wherein:
Ring A is a 5-6 membered heteroaryl or a phenyl;
each R¹ is independently halogen, azido, or C1-C6 alkoxy, wherein not more than one of R¹ is azido;
m is 2, 3, 4, or 5;
each R² is independently halogen, C1-C6 alkyl, or C1-C6 haloalkyl;
n is 0, 1, 2, 3, or 4;
R³ is —OH, —O(C1-C6 alkyl), —NHR^{A}, or —NHR^{B};
R^{A} is hydrogen or C1-C6 alkyl;
R^{B} is

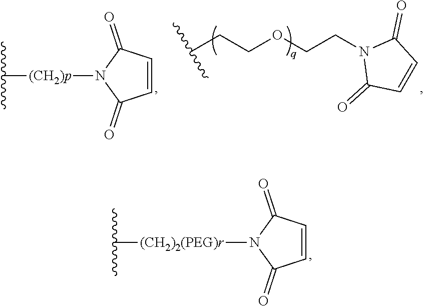

—(CH2)s-X, or -(PEG)t-X;
X is an electrophilic group;
p, q, r, s, and t are each an independently selected integer from 2-20; and
D is a Drug Moiety.

Some embodiments provide a pharmaceutical composition comprising a compound of Formula (I), or a pharmaceutically acceptable salt thereof, and one or more pharmaceutically acceptable excipients.

Some embodiments provide a pharmaceutical composition comprising a compound of Formula (II), or a pharmaceutically acceptable salt thereof, and one or more pharmaceutically acceptable excipients.

Some embodiments provide a method of treating a disease or disorder in a subject in need thereof, comprising administering to the subject a therapeutically effective amount of a compound of Formula (I), o a pharmaceutically acceptable salt thereof, or a pharmaceutical composition as described herein, and administering to the subject an effective amount of radiation. In some embodiments, the disease or disorder is cancer.

The details of one or more embodiments of the present disclosure are set forth in the accompanying drawings and the description below. Other features, objects, and advantages of the present disclosure will be apparent from the description and drawings, and from the claims.

### DESCRIPTION OF DRAWINGS

FIG. 1 Illustrates a radiation cleavable drug-conjugate, wherein drug payloads are conjugated to a therapeutic protein, polymer, peptide, or other material, which is then delivered to a patient, and then released upon exposing the patient to ionizing radiation.

FIG. 2. Illustrates radiation-cleavable linkers and measured release rates of peptide drugs. Left: chemical structures of MMAE prodrugs that are based on the “tri-dendate self-immolative linker” design, but incorporating radiation-labile moieties DMBA, pATFB, and DABA. Right: prodrugs with the DMBA, DABA, or pATFB caging moieties release active drug payload in an oxygen-dependent manner that is enhanced under hypoxia with a vacuum (middle) and especially when oxygen is purged with an inert gas (Argon, right).

FIG. 3. Illustrates drug release initiation via radical hydroxylation under hypoxia. (A) Radiation-induced radical hydroxylation, followed by 1,6-elimination and subsequent loss of SIL caging group, releases the caged drug payload. (B) Measured MMAE release from DMBA-SIL-MMAE following 8 Gy irradiation with either an X-ray or gamma ray source, as a function of oxygen partial pressure. Data are means+/−s.e. (n=3). Pearson's correlation coefficient and two-tailed t-test are reported.

FIG. 4. Illustrates MMAE release from DMBA-SIL-Mal after degassing and X-ray irradiation. (A) Relative amount of MMAE released from irradiated DMBA-SIL-Mal-MMAE prodrug with varying X-ray irradiation, as determined by LC-MS quantification (AUC, area under curve of 3). (B) LC-MS chromatographs (isolated +ESI mass detection of 718.8 Da) of intact prodrug, irradiated prodrug, and free MMAE; (C) Release of free drugs and intact prodrugs after incubation in PBS at 37° C., pH 7.4, in absence of X-ray irradiation. Released drug levels measured 4 hours post-irradiation are shown for reference.

FIG. 5. Illustrates drug payload stability after X-ray irradiation. LC-MS chromatographs demonstrate the lack of byproducts of MMAE (A) and doxorubicin (B) after X-ray irradiation (8 Gy) under degassing. Both drug solutions were prepared to a concentration of 100 uM in PBS and analyzed before and after irradiation. MMAE chromatographs were obtained via ELSD and DOX chromatographs were obtained via UV absorbance scan (200-800 nm).

FIG. 6. Illustrates the identification of release intermediates in a solution of DMBA-SIL-MMAE 1 h after X-ray irradiation (8 Gy). (A) Scheme showing the anticipated intermediates upon radiation-mediated hydroxylation. (B) Representative isolated mass chromatographs (+ESI) with labeled peaks of the corresponding structures. The ion detection signals for the intermediates (1135.8 and 925.8 Da) were graphically increased by a factor of 10 to be more easily observed in comparison to the intact prodrug and the released MMAE. (C) Percent of released MMAE detected by LC-MS 1 hour post-irradiation with 8 Gy X-rays. Data are means+/−s.e., n=3.

FIG. 7. Illustrates dose-response of payload release from drug-albumin conjugates. (A) Percentage of MMAE and DOX released from albumin-conjugates after treatment with varying doses of X-ray irradiation compared to free drug (10 μM). Data are means+/−s.e. (n=3). Representative LC-MS chromatograph (B) MMAE (+ESI, isolated mass of 718.8 Da) and (C) DOX (+ESI, isolated mass of 544.4) released from albumin.

FIG. 8. Illustrates caged and conjugated MMAE is selectively cytotoxic and activated by X-ray irradiation. (A) Chemical structures of Alb-DMBA-SIL-MMAE and other radiation-activated derivatives. (B) Cytotoxicity of un-irradiated and irradiated (8 Gy) MMAE and prodrug derivatives in anaplastic thyroid cancer cells (8505c), measured 72 hours post-treatment by a resazurin-based assay (data are means+/−s.e., n=4); MMAE (triangles pointing down), DMBA-MMAE (triangles pointing up), DMBA-SIL-MMAE (squares), and Alb-DMBA-SIL-MMAE (circles). (C) Half-maximal inhibitory concentration (IC₅₀) of unirradiated or irradiated Alb-DMBA-SIL-MMAE in cancer cell lines of anaplastic thyroid cancer (TBP), oral squamous cell carcinoma (MOC-2), colon adenocarcinoma (MC38), and pancreatic adenocarcinoma (iKRAS).

FIG. 9. Illustrates radiation restores microtubule-disrupting activity of caged MMAE. (A) A reporter cell line for tracking +TIP (microtubule plus-end tracking protein) was imaged over time via confocal microscopy to visualize microtubule dynamics. After drug treatment, microtubule “comets” were automatically detected, computationally tracked, and visualized with pseudo-coloring according to comet speed. (B) Corresponding to representative data in (A), features of microtubule dynamics were averaged across individual cells (n>10 per condition). Prodrug=Alb-DMBA-SIL-MMAE; n.d.=none (no comets) detected.

FIG. 10. Illustrates caged and conjugated DOX is selectively cytotoxic and activated by X-ray irradiation. (A) Structures of DOX prodrug derivatives. (B) Cytotoxicity of each prodrug/conjugate: DOX (triangles pointing down), DMBA-SIL-DOX (squares), and Alb-DMBA-SIL-DOX (circles), +/−8 Gy X-ray irradiation in comparison to free DOX in anaplastic thyroid cancer cells (8505c). Data are means+/−s.e., n=4. (C) Half-maximal inhibitory concentration (IC₅₀) of unirradiated or irradiated Alb-DMBA-SIL-DOX in cancer cell lines of anaplastic thyroid cancer (TBP), oral squamous cell carcinoma (MOC-2), and colon adenocarcinoma (MC38).

FIG. 11 Illustrates intrinsic doxorubicin fluorescence quantifies nuclear uptake following X-ray mediated payload release. (A) Representative images of intrinsic DOX fluorescence in 8505c cells treated with DOX, non-irradiated Alb-DMBA-SIL-DOX, or X-ray irradiated (8 Gy) conjugate for 24 hours; Hoechst 33342 counterstains cell nuclei. (B) The ratio of nuclear to cytoplasmic DOX fluorescence was quantified to evaluate the subcellular drug accumulation. Data are means+/−s.e., n>15 single-cells per condition (One-way ANOVA with Dunnett's T3 multiple comparisons test).

FIG. 12. Illustrates radiation-mediated release of antibody-drug-conjugate (ADC) payloads. (A) Chemical structures of ADCs, here using anti-EGFR mAb as a model tumor-targeted antibody. (B) MMAE and DOX payload release from their respective antibody conjugates after X-ray irradiation (8 Gy), measured by LC-MS. Data are means+/−s.e., n=3. (C) Cytotoxicity of non-irradiated or X-ray irradiated (8 Gy) mAb-DMBA-SIL-MMAE conjugate in comparison to free MMAE in anaplastic thyroid cancer (8505c cell line). (D) The ratio of nuclear to cytoplasmic DOX fluorescence was quantified as in FIG. 8 in anaplastic thyroid cancer cells (8505c) treated for 24 hours with either DOX, non-irradiated conjugate, or irradiated (8 Gy) conjugate. Prodrug=mAb-DMBA-SIL-DOX. Data are means+/−s.e., n>16 single-cells per condition (One-way ANOVA with Dunnett's T3 multiple comparisons test).

FIG. 13. Illustrates radiation-activatable prodrugs of MMAE, DOX, and Gard having a tridentate self-immolative linker. Maleimide-functionalized compounds 11 and 13 were conjugated to serum albumin.

FIG. 14. Illustrates stability of radiation-activatable prodrugs with and without radiation exposure. (A) Stability of prodrugs pATFB-SIL-MMAE, pATFB-SIL-DOX, and pATFB-SIL-Gard while incubating at 37° C. in PBS (pH 7.4) for varying time intervals; (B) Drug released from pATFB-SIL-MMAE in various media including PBS, DMEM, and fetal bovine serum.

FIG. 15. Illustrates prodrug activation via proton beam irradiation. DOX release from pATFB-DOX and DMBA-DOX was measured after proton beam irradiation via either conventional or FLASH dose rates.

FIG. 16. Illustrates prodrug activation via multiple methods. (A) Release of each drug from its respective pATFB-SIL prodrug upon X-ray irradiation; (B) MMAE released from pATFB-SIL-MMAE via various irradiation conditions.

FIG. 17. Illustrates prodrug activation with and without self-immolative-linker (SIL), and using X-ray versus radionuclide. (A) Chemical structures of pATFB-prodrug and pATFB-SIL-prodrug design. (B) MMAE release following X-ray irradiation for pATFB-MMAE and pATFB-SIL-MMAE. (C) MMAE release following incubation with 300 μCi Cu-64.

FIG. 18. Illustrates prodrug activation in live cells and tumor tissues. (A) MMAE released from Alb-pATFB-SIL-MMAE in live HT1080 cancer cells in culture, after 24 hour incubation followed by irradiation, cell lysis, LC-MS quantification. As a control, uncaged MMAE was spiked into cell lysate and quantified. (B) Quantification of MMAE release from Alb-pATFB-SIL-MMAE in irradiated tumors. Female C57Bl/6 mice bearing subcutaneous MC38 tumors were treated i.v. with 10 mg/kg (MMAE eq.) Alb-pATFB-SIL-MMAE. Tumors were irradiated with 10 Gy X-ray, excised, and analyzed for MMAE release by LC/MS. (C) Drug release in cell culture, following 24 hr prodrug treatment and 1 hr after a 10 Gy X-ray exposure.

FIG. 19. Illustrates chemical activation and cellular immunostimulation of caged toll like receptor agonist. (A) Gardiquimod released from Alb-pATFB-SIL-Gard following X-ray irradiation, with TCEP reduction used as a positive control. (B-F) 100 nM Gardiquimod or Compound 10 were added to BMDM isolated from IL12-eYFP expressing mice. After plating, either (A) gardiquimod, (B) prodrug 10, or (C) TCEP activated (10 uM) prodrug 10 were added to the cells, which were then incubated at 37 C for 48 hours. The cells were then fixed and stained for p-NFkB prior to imaging (DV) and the mean fluorescence intensities of both p-NFkB (D) and IL12-eYFP (E) were determined using FIJI, and plotted using Graphpad Prism (n>50). FIG. 20. Illustrates that MMAE conjugate is selectively cytotoxic in multiple cancer cell lines including from anaplastic thyroid cancer (TBP), oral squamous cell carcinoma (MOC2), colon adenocarcinoma (MC38), and pancreatic adenocarcinoma (iKRAS) cancer types. Cytotoxicity was determined at the indicated concentration after a 72 hour treatment using a resazurin-based assay (PrestoBlue). Data are means+/−s.e., n=3.

FIGS. 21A-C. Illustrate DOX conjugate is selectively cytotoxic in multiple cancer cell lines including anaplastic thyroid (TBP), oral (MOC2), and colon (MC38) cancer types. Cytotoxicity was determined at the indicated concentration after a 72 hour treatment using a resazurin-based assay (PrestoBlue). Data are means+/−s.e., n=3.

FIG. 22. Illustrates Subcellular distribution of DOX in 8505c cells treated with either DOX, non-irradiated conjugate, or irradiated conjugate, shown as means (thick line)±s.e.m (shading; n>10 cells), for Hoechst 33342 DNA/nuclear counterstain (blue) and DOX (red). Data correspond to FIG. 11.

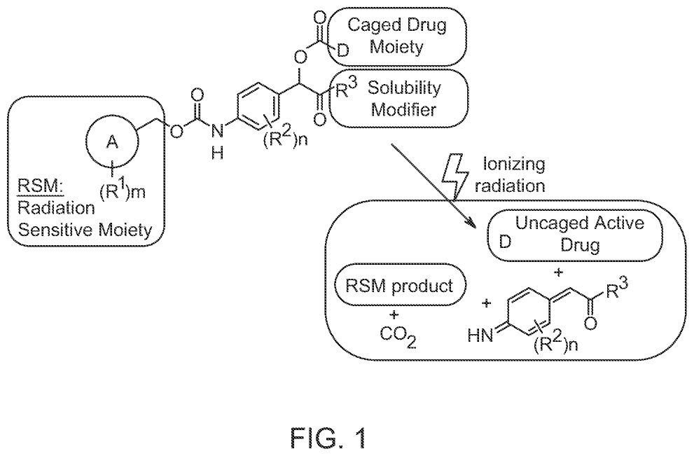

### DETAILED DESCRIPTION

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In case of conflict, the present specification, including definitions, will control.

The term “about” when referring to a number or a numerical range means that the number or numerical range referred to is an approximation, for example, within experimental variability and/or statistical experimental error, and thus the number or numerical range may vary up to +10% of the stated number or numerical range.

The phrase “therapeutically effective amount” means an amount of compound that, when administered to a subject in need of such treatment, is sufficient to (i) treat the indicated disease or disorder, (ii) attenuate, ameliorate, or eliminate one or more symptoms of the particular disease or disorder, or (iii) delay the onset of one or more symptoms of the particular disease or disorder described herein.

An “effective amount” as used herein with respect to an amount of radiation administered to a subject, is an amount of radiation sufficient to induce the breakdown of a compound of Formula (I) to release a drug, as described herein. In some embodiments, an effective amount of radiation is a sub-therapeutic amount. In some embodiments, the effective amount of radiation is a therapeutically effective amount.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halogen” refers to fluoro (F), chloro (Cl), bromo (Br), or iodo (I).

The term “alkyl” refers to a saturated acyclic hydrocarbon radical that may be a straight chain or branched chain, containing the indicated number of carbon atoms. For example, C1-10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of

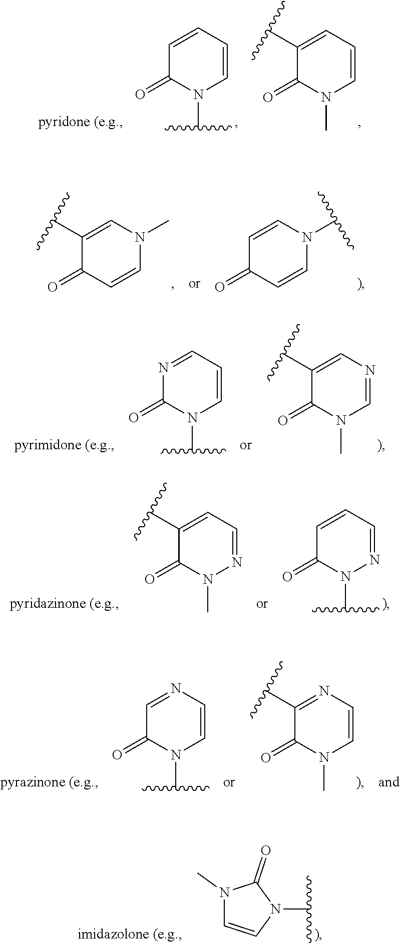

wherein each ring nitrogen adjacent to a carbonyl is tertiary (i.e., the oxo group (i.e., “═O”) herein is a constituent part of the heteroaryl ring).

As used herein, examples of aromatic rings include: benzene, pyridine, pyrimidine, pyrazine, pyridazine, pyridone, pyrrole, pyrazole, oxazole, thioazole, isoxazole, isothiazole, and the like.

For the avoidance of doubt, and unless otherwise specified, for rings and cyclic groups containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

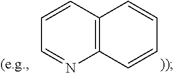

(ii) a single ring atom (spiro-fused ring systems)

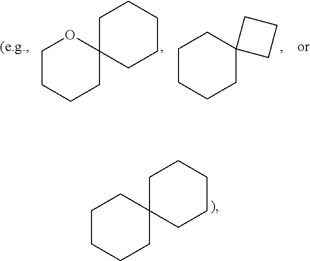

or (iii) a contiguous array of ring atoms (bridged ring systems having all bridge lengths >0)

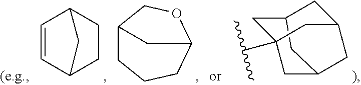

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

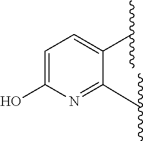 [SMILES: *c1ccc(O)nc1*]

encompasses the tautomeric form containing the moiety:

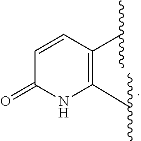 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### Introduction

It has generally been a challenge to achieve highly localized control of drug activation deep through tissue in a non-invasive manner Methods such as heat, ultrasound, and laser irradiation may not easily penetrate deep through tissue, and bio-orthogonal chemical triggers with increased selectivity and compatibility are still in development. Ionizing radiation offers an attractive solution: radiation is routinely delivered deep through tissue via focused beams of gamma, proton, and X-ray radiation.

The present disclosure is directed, inter alia, to compounds with a radiation-activated trigger, a linker, a cytotoxic payload, and optionally a solubility modifier.

The present disclosure relates to a tri-dentate prodrug approach to the use of ionizing radiation as a “trigger” to release active drug. As described and demonstrated in the present disclosure, this linking approach leads to a dramatic impact on caging efficiency compared to the small-molecule prodrug designs previously published, even in vitro. These improvements are anticipated to be more pronounced in vivo, as they will improve drug pharmacokinetics and tumor accumulation (as has been extensively reported for traditional NPs and ADCs). Since radiation treatments can be designed to avoid sites of off-target toxicity, in principle the linking approach described herein offers the possibility of activating drug only at intended target sites: primary and disseminated tumors.

### Radiation-Sensitive Moiety

A radiation-sensitive moiety is one that upon exposure to radiation undergoes a chemical transformation in such a manner as to promote release the Drug Moiety.

In some embodiments, the radiation-sensitive moiety is 4-hydroxymethyl-2,3,5,6-tetrafluoroaryl azide (pATFB), (3,5-bis(dimethylamino)phenyl) methanol (DABA), or 3,5-dimethyloxybenzyl alcohol (DMBA). Alternative Radiation-sensitive Moieties include, for example, quaternary ammonium compounds such as those disclosed in Guo Z, et al. Angew Chem Int Ed Engl. 2022, Vol. 61, which is hereby incorporated by reference in its entirety.

### Solubility Modifier

Solubility Modifiers refer to chemical moieties that may either increase or decrease the solubility of Formula (I), as needed. Exemplary Solubility Modifiers include, but are not limited to chemical groups designed to bind serum albumin, extracellular matrix proteins, or other reactive groups that would form a covalent bond to a peptide, protein, or other cellular component. For example, when a compound of Formula (I) comprising a Solubility Modifier is introduced into an in vitro, in vivo, or animal system (e.g., mouse, human, and the like), the Solubility Modifier can form a covalent bond to a peptide, protein, or other cellular component. In some embodiments, the Solubility Modifier is a maleimide group.

### Drug Moiety

Compounds of Formula (I) can incorporate a diverse range of D-groups, such as drugs, materials, proteins, sugars, nucleic acids, and other biologically active materials.

In some embodiments, the Drug Moiety (D) is a cytotoxic, cytostatic or immunomodulatory agent. The Drug Moiety has a nitrogen atom that can form a bond with the Linker, as described herein.

In some embodiments, the Drug Moiety (D) is selected from antitubulin agents, DNA replication inhibitors, alkylating agents, antifolates, antimetabolites, chemotherapy/radiotherapy sensitizers, topoisomerase inhibitors, and vinca alkaloids.

In some embodiments, the Drug Moiety (D) is selected from DNA replication inhibitors, alkylating agents, and tubulin inhibitors.

In some embodiments, the antitubulin agent (also referred to as a tubulin inhibitor) is, for example,

In some embodiments, the DNA replication inhibitor is, for example, altretamine, bleomycin, cytarabine, dacarbazine, dactinomycin, ellipticine, estramustine, mitomycin, plicamycin, or temozolomide.

In some embodiments, the alkylating agent is, for example, cisplatin, oxaliplatin, carboplatin, cyclophosphamide, nitrogen mustards, bendamustine, carmustine, melphalan, chlorambucil, busulphan, temozolamide, or nitrosoureas.

In some embodiments, the antifolate is, for example, methotrexate, trimethoprim, brodimoprim, tetroxoprim, iclaprim, pemetrexed, ralitrexed, or pralatrexate.

In some embodiments, the antimetabolite is, for example, gemcitabine.

In some embodiments, the chemotherapy/radiotherapy sensitizer is, for example, gemcitabine, interferon-α, 13-cis-retinoic acid, doxorubicin, docetaxel, carboplatin, cisplatin, dactinomycin, methotrexate, 5-fluorouracil, bleomycin, or hydroxyurea.

In some embodiments, the topoisomerase inhibitor is, for example, etoposide, teniposide, amsacrine, topotecan, mitoxantrone, or camptothecin.

In some embodiments, the vinca alkaloid is, for example, vincristine, vinblastine, vindesine, and vinorelbine.

Additional exemplary cytotoxic agents include, for example, auristatins, camptothecins, duocarmycins, etoposides, maytansines and maytansinoids, taxanes, benzodiazepines or benzodiazepine containing drugs (e.g., pyrrolo[1,4]-benzodiazepines (PBDs), indolinobenzodiazepines, and oxazolidinobenzodiazepines) and vinca alkaloids. Select benzodiazepine containing drugs are described in WO 2010/091150, WO 2012/112708, WO 2007/085930, and WO 2011/023883.

In some embodiments, the cytotoxic agent is maytansine or a maytansinoid (e.g., DM1, DM4) another group of anti-tubulin agents.

In some embodiments, the Drug Moiety is a benzodiazepine (including benzodiazepine containing drugs e.g., pyrrolo[1,4]benzodiazepines (PBDs), indolinobenzodiazepines, and oxazolidinobenzodiazepines).

In some embodiments, D is monomethyl auristatin E (MMAE) or doxorubicin (DOX). In some embodiments, D is monomethyl auristatin E (MMAE). In some embodiments, D is doxorubicin (DOX). In some embodiments, D is gardiquimod (Gard).

### Linker

The Linker is a group that bridges the Radiation-sensitive Moiety and the Drug Moiety, and that releases the drug payload upon radiation-induced reaction. In some embodiments, the Linker is a 2-acetoxy-2-(4-aminophenyl) acetyl moiety or a 3,5-dihydroxybenzyl carbamate moiety. Additional linkers include, but are not limited to those disclosed in Miller, M. A. et al. ACS nano 2018, Vol. 12, pp. 12814-12826 and Fu, Q. et al. Angew. Chem. Int. Ed. 2020, Vol. 59, pp. 21546-21552), which are hereby incorporated by reference in their entireties.

### Compounds of Formula (I)

Some embodiments provide a compound of Formula (I), or a pharmaceutically acceptable salt thereof,

[RSM]-Linker-Drug Moiety (I)
wherein RSM is a radiation-sensitive moiety.

In some embodiments, the Linker comprises a carbamate group and an optional solubility modifier.

Some embodiments provide a compound of Formula (I-A), or a pharmaceutically acceptable salt thereof,

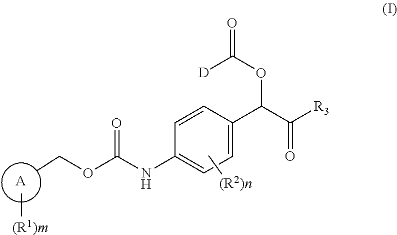

wherein:
Ring A is a 5-6 membered heteroaryl or a phenyl;
each R¹ is independently halogen, azido, or C1-C6 alkoxy, wherein not more than one of R¹ is azido;
m is 2, 3, 4, or 5;
each R² is independently halogen, C1-C6 alkyl, or C1-C6 haloalkyl;
n is 0, 1, 2, 3, or 4;
R³ is —OH, —O(C1-C6 alkyl), —NHR^{A}, or —NHR^{B};
R^{A} is hydrogen or C1-C6 alkyl;
R^{B} is

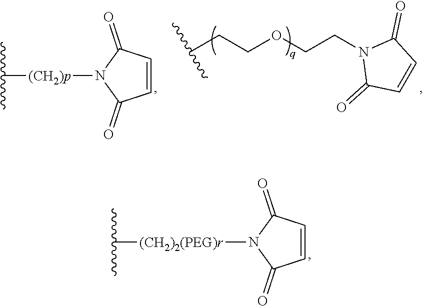

—(CH2)s-X, or -(PEG)t-X;
X is an electrophilic group;
p, q, r, s, and t are each an independently selected integer from 2-20; and D is a Drug Moiety.

In some embodiments, Ring A is phenyl.

In some embodiments, Ring A is a 5-6 membered heteroaryl. In some embodiments, Ring A is a 5 membered heteroaryl. In some embodiments, Ring A is a 6 membered heteroaryl.

In some embodiments, m is 2. In some embodiments, m is 3. In some embodiments, m is 4. In some embodiments, m is 5. In some embodiments, m is 2 or 5.

In some embodiments, m is 2 and each R¹ is independently halogen, azido, or C1-C6 alkoxy, wherein not more than one of R¹ is azido.

In some embodiments, m is 3 and each R¹ is independently halogen, azido, or C1-C6 alkoxy, wherein not more than one of R¹ is azido.

In some embodiments, m is 4 and each R¹ is independently halogen, azido, or C1-C6 alkoxy, wherein not more than one of R¹ is azido.

In some embodiments, m is 5 and each R¹ is independently halogen, azido, or C1-C6 alkoxy, wherein not more than one of R¹ is azido.

In some embodiments, m is 2 and each R¹ is independently halogen, azido, or methoxy, wherein not more than one of R¹ is azido.

In some embodiments, m is 3 and each R¹ is independently halogen, azido, or methoxy, wherein not more than one of R¹ is azido.

In some embodiments, m is 4 and each R¹ is independently halogen, azido, or methoxy, wherein not more than one of R¹ is azido.

In some embodiments, m is 5 and each R¹ is independently halogen, azido, or methoxy, wherein not more than one of R¹ is azido.

In some embodiments, m is 2 and each R¹ is methoxy.

In some embodiments, m is 5, one R¹ is azido, and the remaining R¹ are each fluoro.

In some embodiments, n is 0. In some embodiments, n is 0 or 1. In some embodiments, n is 1. In some embodiments, n is 2. In some embodiments, n is 3. In some embodiments, n is 4.

In some embodiments, each R² is independently halogen.

In some embodiments, n is 1 and R² is halogen, C1-C6 alkyl, or C1-C6 haloalkyl.

In some embodiments, n is 2 and each R² is independently halogen, C1-C6 alkyl, or C1-C6 haloalkyl.

In some embodiments, n is 3 and each R² is independently halogen, C1-C6 alkyl, or C1-C6 haloalkyl.

In some embodiments, n is 4 and each R² is independently halogen, C1-C6 alkyl, or C1-C6 haloalkyl.

In some embodiments, n is 1 and R² is fluoro, chloro, methyl, or trifluoromethyl.

In some embodiments, n is 2 and each R² is independently fluoro, chloro, methyl, or trifluoromethyl.

In some embodiments, n is 3 and each R² is independently fluoro, chloro, methyl, or trifluoromethyl.

In some embodiments, n is 4 and each R² is independently fluoro, chloro, methyl, or trifluoromethyl.

In some embodiments, R³ is —NHR^{B}.

In some embodiments, R^{B} is

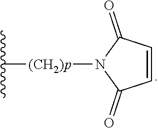

In some embodiments, R^{B} is

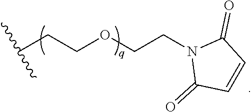

In some embodiments, R^{B} is

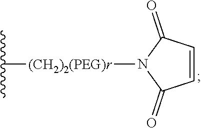

In some embodiments, R^{B} is —(CH2)s-X or -(PEG)t-X. In some embodiments, R^{B} is —(CH2)s-X. In some embodiments, R^{B} is -(PEG)t-X.

In some embodiments, X is an electrophilic group. Exemplary electrophilic groups include, but are not limited to aldehydes, ketones (such as a/β unsaturated, α-halo, and α-haloalkyl ketones), and esters (such as NHS and pentafluorophenyl esters), and the like.

In some embodiments, R³ is —OH.

In some embodiments, R³ is —O(C1-C6 alkyl).

In some embodiments, R³ is —NHR^{A}.

In some embodiments, R^{A} is hydrogen. In some embodiments, R^{A} is C1-C6 alkyl.

In some embodiments, p is an integer from 2-20, from 2-16, from 2-12, from 2-10, from 2-8, or from 2-4. In some embodiments, p is 2, 4, 8, or 12.

In some embodiments, q is an integer from 2-20, from 2-16, from 2-12, from 2-10, from 2-8, or from 2-4. In some embodiments, q is 2, 4, 8, or 12.

In some embodiments, r is an integer from 2-20, from 2-16, from 2-12, from 2-10, from 2-8, or from 2-4. In some embodiments, r is 2, 4, 8, or 12.

In some embodiments, s is an integer from 2-20, from 2-16, from 2-12, from 2-10, from 2-8, or from 2-4. In some embodiments, s is 2, 4, 8, or 12.

In some embodiments, t is an integer from 2-20, from 2-16, from 2-12, from 2-10, from 2-8, or from 2-4. In some embodiments, tis 2, 4, 8, or 12.

### Exemplary Compounds of the Disclosure

Exemplary compounds of Formula (I) include, but are not limited to DMBA-SIL-Mal-MMAE (7), DMBA-SIL-Mal-DOX (9), and pATFB-SIL-Mal-MMAE (11), and pATFB-SIL-Mal-Gard (13).

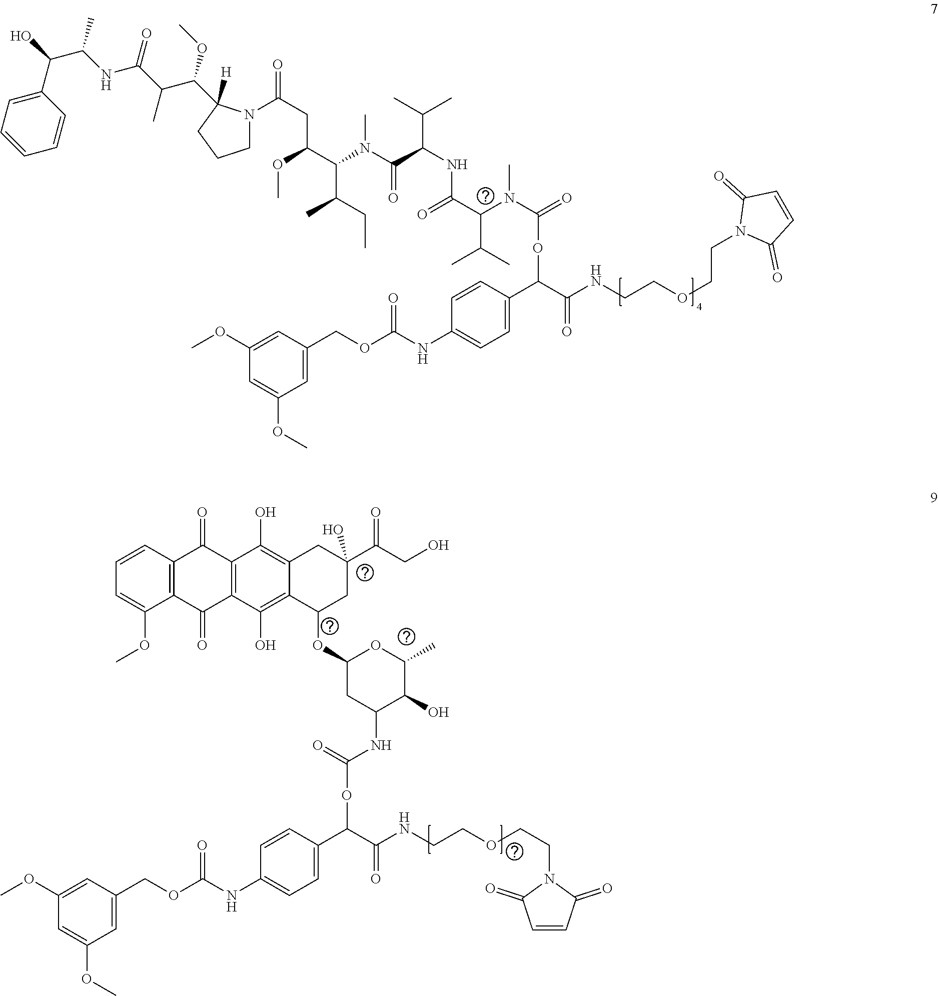

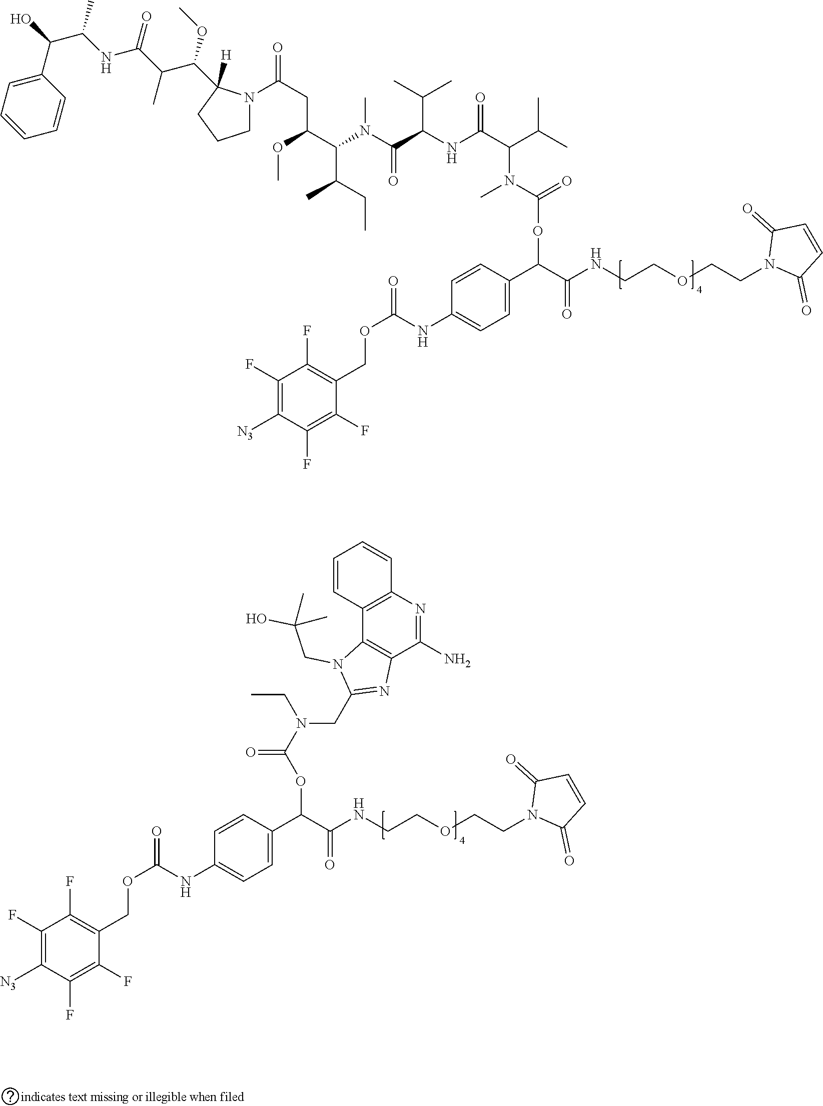

### Radiation

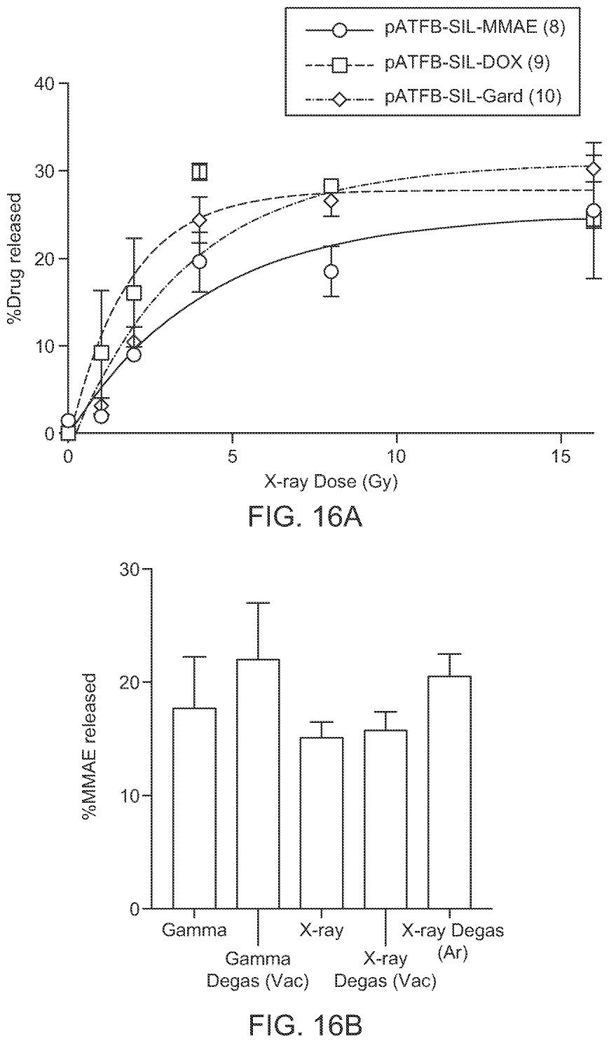

Radiation at the doses described herein will be primarily relevant for oncology, but some non-malignant diseases are also treated with lower doses of radiation. Activation of the compounds described herein can be achieved with various sources of radiation and via multiple methods. As non-limiting examples shown in FIG. 16, drug payloads can be released from pATFB-SIL compounds upon X-ray irradiation under 8 different irradiation conditions.

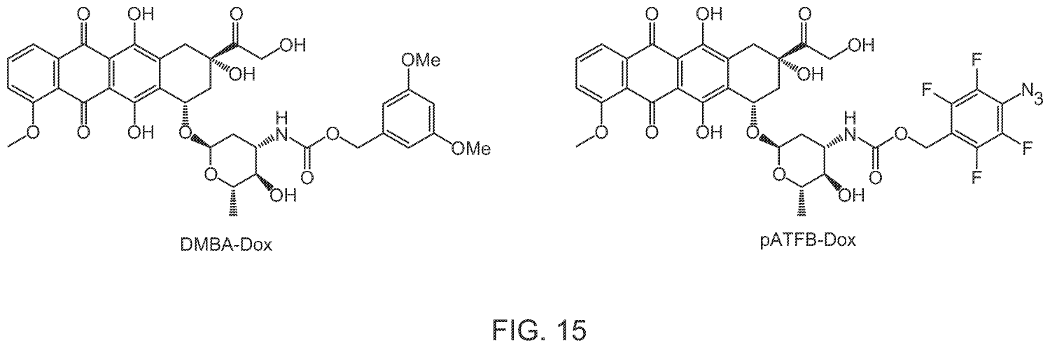

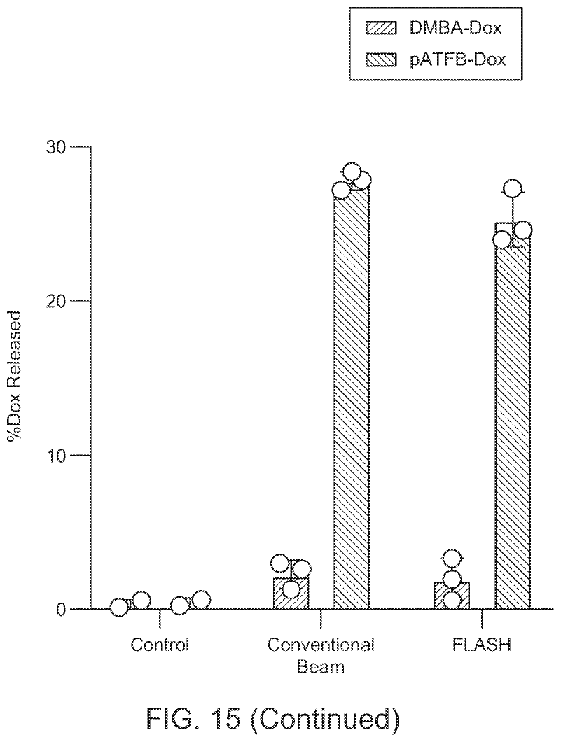

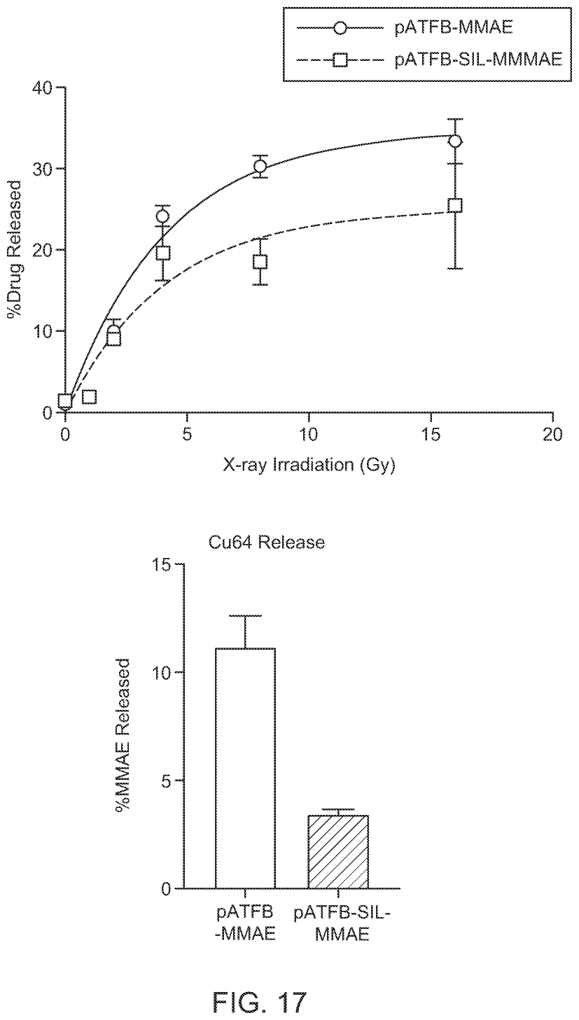

In some embodiments, the radiation is X-rays at a dose of about 8 Gy irradiation. In some embodiments, the radiation is proton beam or FLASH high-dose-rate proton beam (e.g., FIG. 15). In some embodiments, the radiation is by exposure to Cu-64 radionuclide. (e.g., FIG. 17)

### Compounds of Formula (II)

Some embodiments provide a compound of Formula (II), wherein a compound of Formula (I), as described herein, comprises a maleimide group that is conjugated to a protein. For example, a thiol group from a cysteine residue on a protein can form a covalent bond with the maleimide group, linking the compound of Formula (I) to the protein, and thus forming a compound of Formula (II).

The Ring A, R¹, R², R³, m, n, o, p, q, r, R^{A}, R^{B}, and D groups of Formula (II) are identical to those used for Formula (I) as described herein, with the exception that the maleimide group forms a covalent bond with a protein, as explained herein.

In some embodiments, the protein is Serum albumin (Alb).

In some embodiments, the protein is an antibody. In some embodiments, the antibody is a therapeutic antibody. In some embodiments, the antibody is a non-therapeutic antibody. In some embodiments, the protein is an EGFR mAb. In some embodiments, the protein is cetuximab or panitumumab. In some embodiments, the protein is cetuximab. In some embodiments, the protein is panitumumab.

### Pharmaceutical Compositions

Some embodiments provide a pharmaceutical composition comprising a compound of Formula (I), or a pharmaceutically acceptable salt thereof, and one or more pharmaceutically acceptable excipients.

Some embodiments provide a pharmaceutical composition comprising a compound of Formula (II), or a pharmaceutically acceptable salt thereof, and one or more pharmaceutically acceptable excipients.

### Methods of Treatment

Some embodiments provide a method of treating a disease or disorder in a subject in need thereof, comprising administering to the subject a therapeutically effective amount of a compound of Formula (I), or a pharmaceutical composition as described herein, and administering to the subject an effective amount of radiation.

Some embodiments provide a method of treating a disease or disorder in a subject in need thereof, comprising administering to the subject a therapeutically effective amount of a compound of Formula (II), or a pharmaceutical composition as described herein, and administering to the subject an effective amount of radiation.

In some embodiments, the disease or disorder is cancer.

In some embodiments, the effective amount of radiation is a therapeutically effective amount of radiation.

In some embodiments, the radiation is ionizing radiation.

In some embodiments, the radiation comprises exposure to an external beam, for example, a proton beam or photon beam.

In some embodiments, the radiation comprises exposure to a radionuclide. Non-limiting examples of radionuclides are Cu-64, Lu-177, and Ac-225.

### Effect of Dissolved Oxygen Levels on Radiation-Induced Cleavage

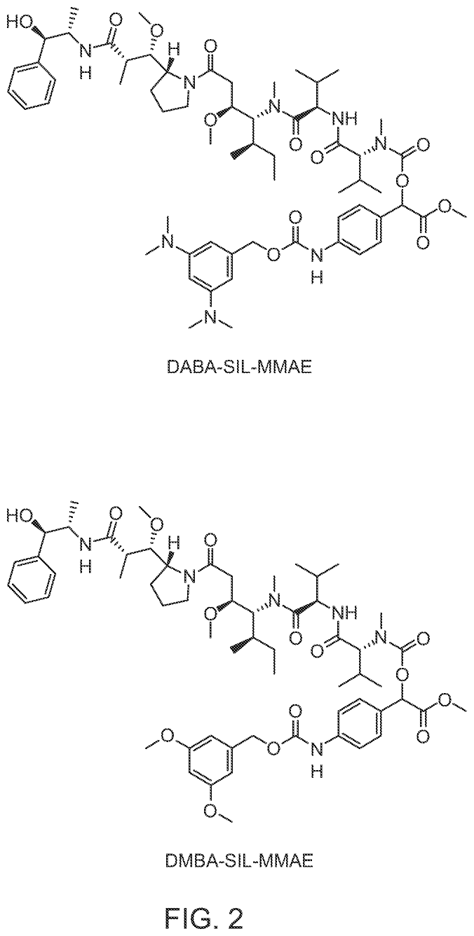

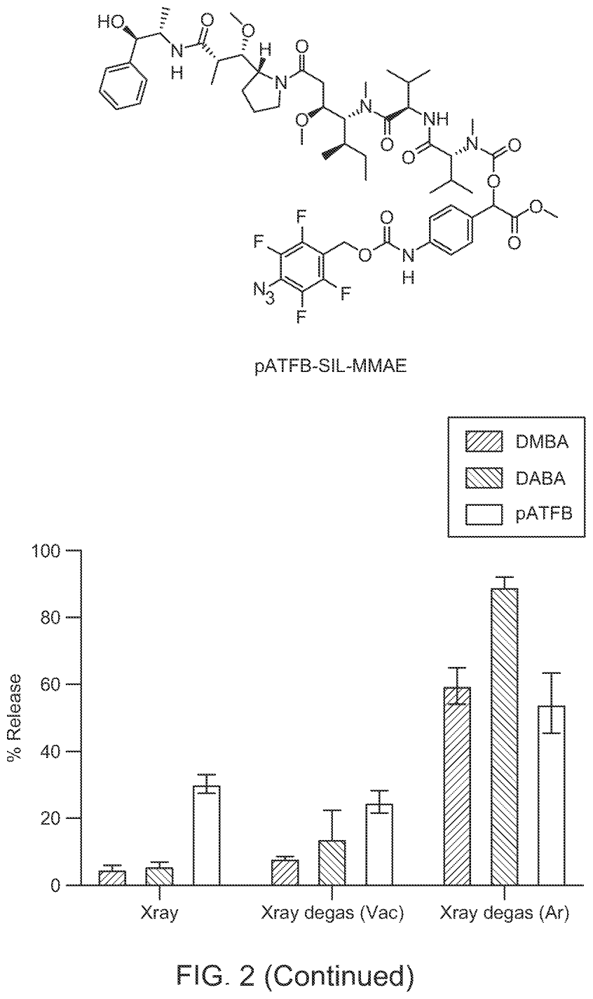

It was discovered that compounds with the DMBA, DABA, or pATFB radiation-sensitive moiety release active drug payload in an oxygen-dependent manner. As shown in FIG. 2, release of drug payload is enhanced under hypoxia. The DMBA and DABA compounds were more sensitive to dissolved oxygen during cleavage compared to pATFB compounds, which suggests that pATFB-containing compounds may perform better under normoxic conditions.

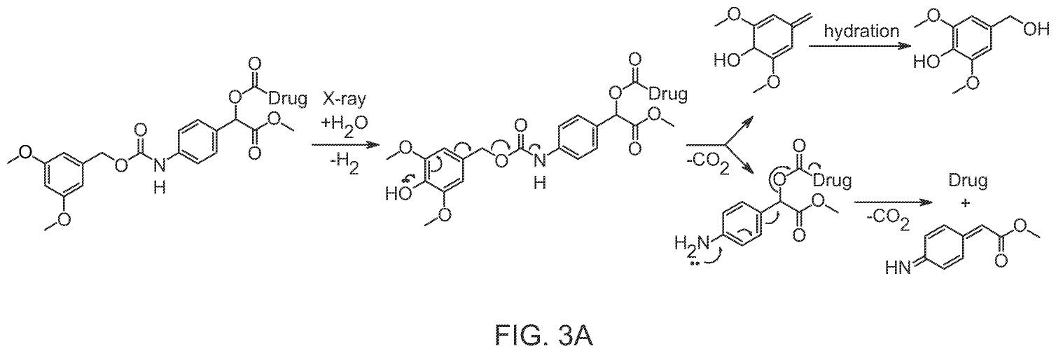

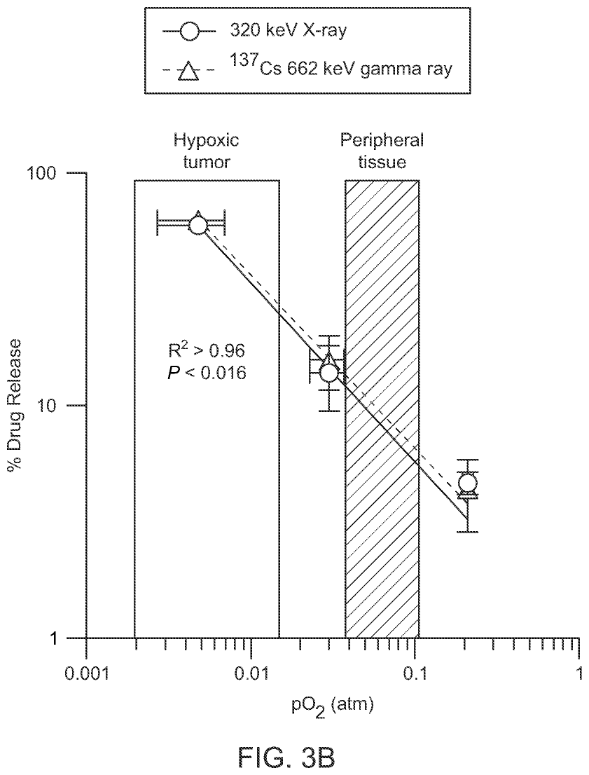

Initial experiments were performed to assess linker cleavage and payload release upon exposure to ionizing radiation. It was hypothesized that the amount of dissolved oxygen in the solution would impact reaction rates, in part due to production of reactive oxygen species (ROS) other than the desired hydroxyl radicals. Although prior literature has hinted at oxygen dependent reaction (Fu, Q. et al. Angew. Chem. Int. Ed. 2020, 59, 21546-21552; Geng, J. et al. Nat. Chem. 2021, 805-810.) there is little data on actual effects. Using previously reported procedures (Butler, I. B. et al. Talanta 1994, 41, 211-215.), dissolved oxygen partial pressure was estimated to be decreased from 0.21 atm under ambient air (21% oxygen), to 0.025 atm with vacuum, to 0.005 atm with inert gas (FIG. 3), as determined by the Winkler titration method. As a reference, dissolved oxygen is proportionate to its partial pressure (pO₂) according to Henry's law, tumor pO₂ values can be >100-fold lower than ambient air pO₂, and some tumor cells and xenografts can survive <0.1% oxygen (Mckeown, S. R. Br. J. Radiol. 2014, 87, 20130676). Thus 0.005 atm oxygen partial pressure achieved by inert gas bubbling (a 40-fold decrease over ambient conditions) is likely relevant to hypoxic tumor tissue.

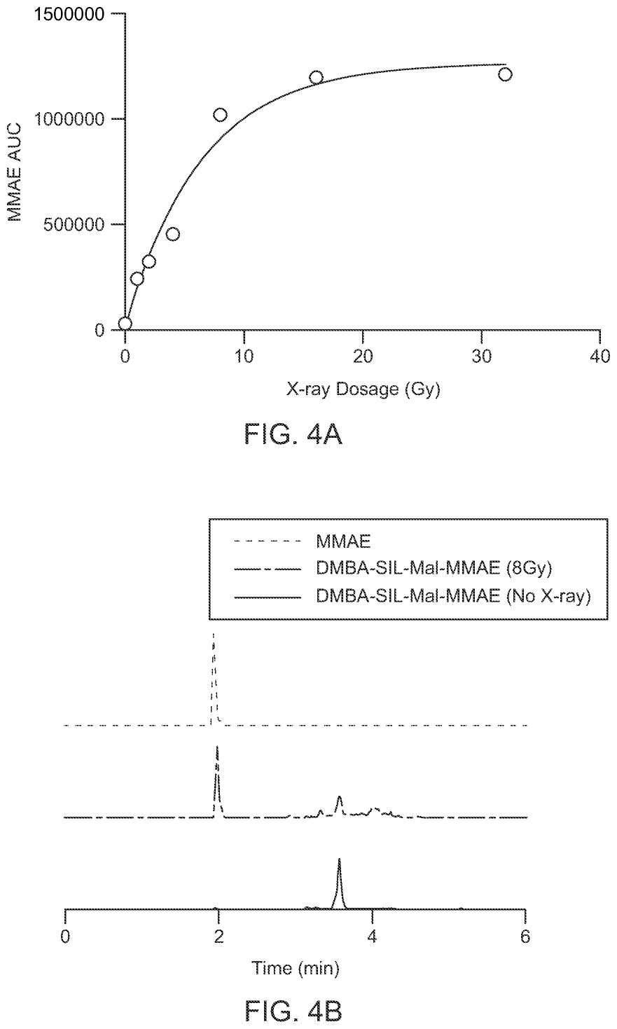

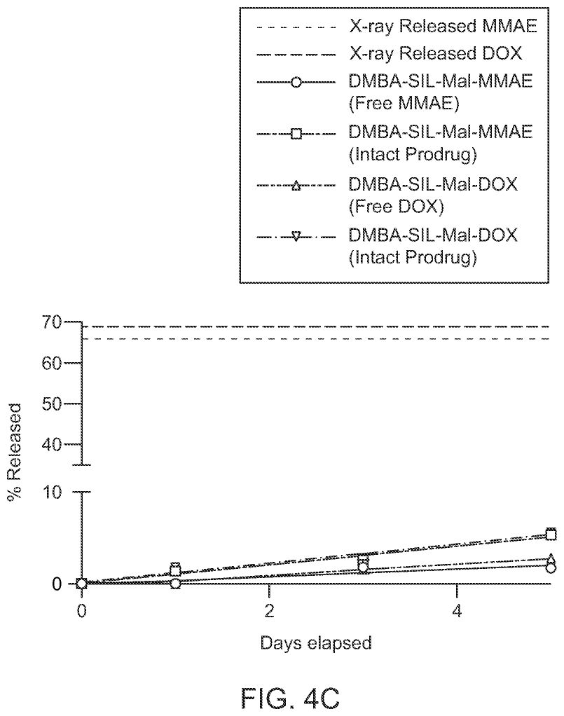

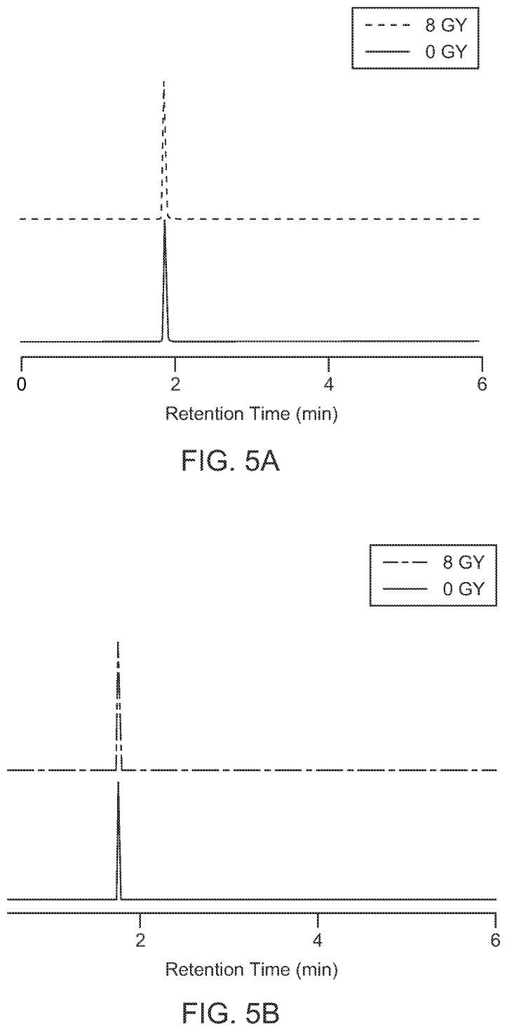

The preceding results indicate a correlation between oxygen levels and the efficiency of linker cleavage (R²>0.97, p=0.02) at a radiation dose of 8 Gy, which is relevant to clinical radiation protocols, especially those using hypofractionated treatment schedules (Rodin, D. et al. Radiother. Oncol. 2021, 157, 32-39; Murray Brunt, A. et al. Lancet 2020, 395, 1613-1626). No difference in cleavage efficiency was found between gamma irradiation from a sealed 137Cs source (primarily 662 keV photons at a dose rate of roughly 0.5 Gy/min), and X-ray irradiation (from a 4000 W tube generating 320 keV photons at 3 Gy/min), and little background release was observed under un-irradiated conditions (FIG. 4). Neither MMAE nor doxorubicin themselves were substantially affected by 8 Gy irradiation, according to LC/MS chromatograph analysis (FIG. 5). These data thus show that radiation can trigger drug-conjugate cleavage in a hypoxia-dependent manner without adversely degrading the drug payload.

The observed dependence on hypoxia may be considered unexpected due to the known role of oxygen in promoting ROS generation. One possible explanation may be that the absence of oxygen enhances the desired hydroxylation mechanism by reducing the quantity of alternative reactive oxygen species being formed, although further exploration of this phenomenon is needed to test this hypothesis and dissect underlying mechanisms.

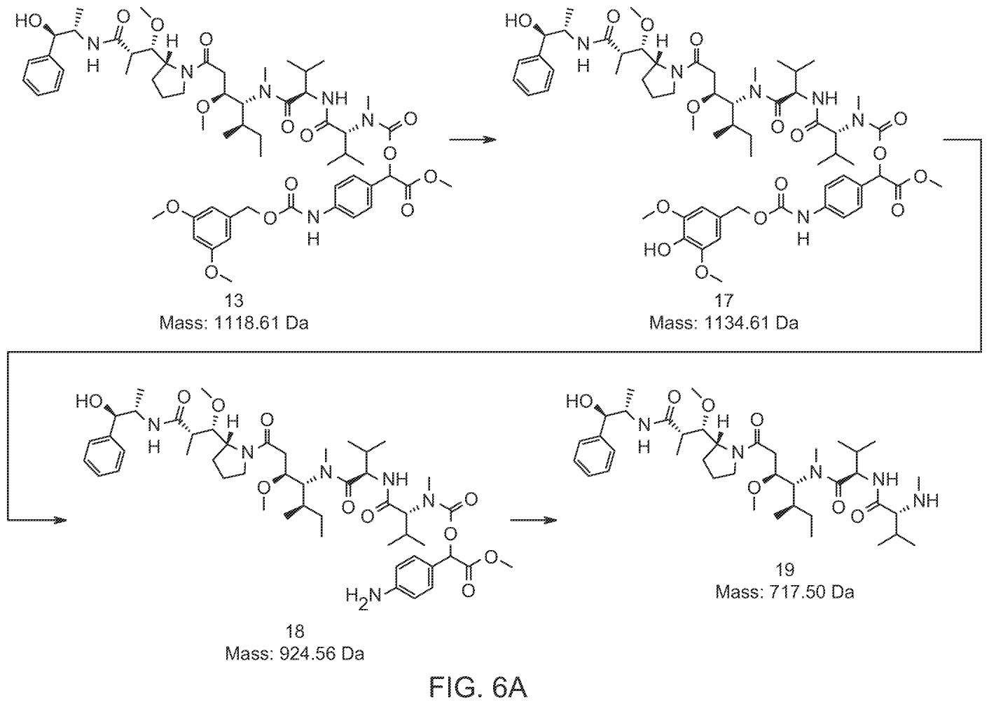

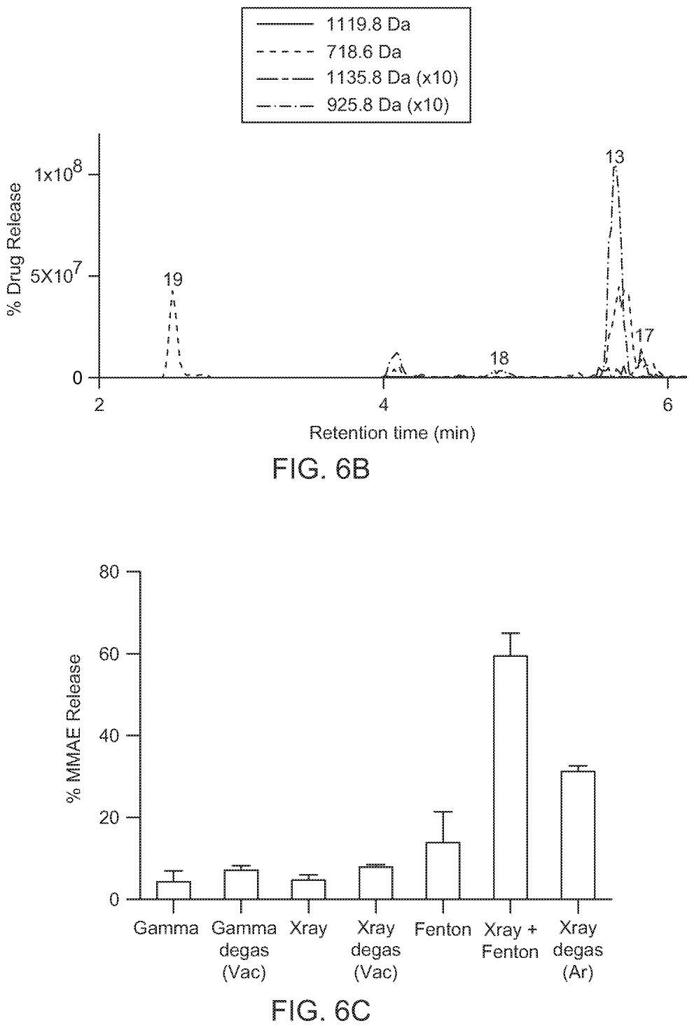

Hydroxyl radicals are thought to be the primary ROS formed from ionizing radiation that activates the DMBA trigger. The mechanisms for the release of doxorubicin from these prodrugs consists of a radical hydroxylation (either position 2 or 4) of the dimethoxybenzyl moiety, followed by a 1-4/1-6 elimination and loss of carbon dioxide. This mechanism is supported by the observed masses of the expected intermediates in our X-ray irradiated prodrug, shortly after irradiation (FIG. 6).

Standard Fenton reaction conditions (100 μM H₂O₂ and 100 μM Fe²⁺) are also known to generate hydroxyl radicals, and therefore can serve as a benchmark comparison to ionizing radiation effects. X-ray irradiation under the most hypoxic conditions was more efficient than Fenton conditions at cleaving the DMBA-based linker (FIG. 6c).

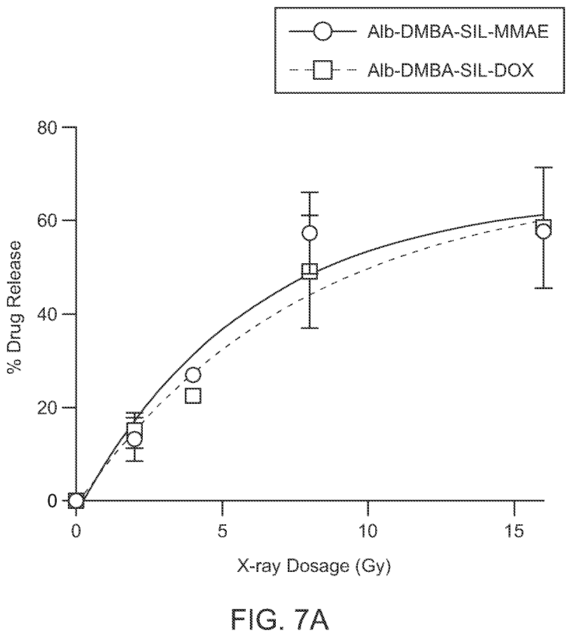

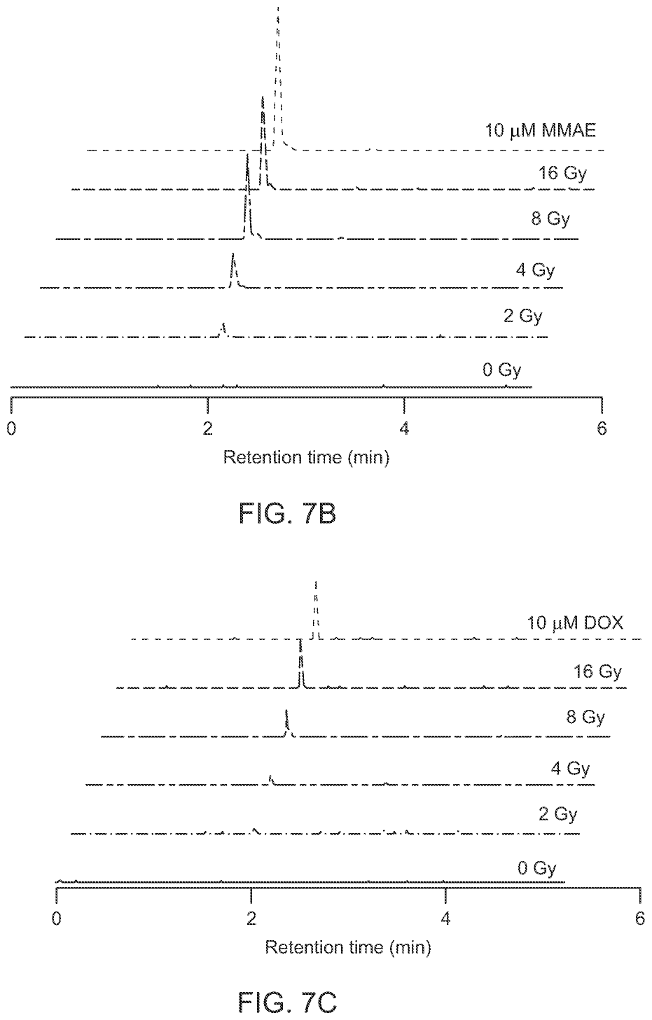

The sensitivity of linker cleavage to varying doses of radiation were examined; Solutions of both conjugates at a concentration of 100 μM were irradiated at an exposure of 1-16 Gy, and LC-MS analysis was performed to determine the released drug concentrations (FIG. 7). Alb-DMBA-SIL-MMAE and Alb-DMBA-SIL-DOX exhibited similar dose-response behaviors, suggesting the approach generalizes to diverse drug payloads. With a standard protocol used in clinical treatments, patients typically receive ˜2 Gy radiation per day, 5 days a week, for ≥5 weeks, therefore yielding cumulative radiation doses of ≥50 Gy. The data presented herein show that even low doses of 1-2 Gy elicit detectable drug release, and higher doses of 8-16 Gy, which are less common but still used clinically in hypofractionated schedules, release 66±6% and 69±8% of MMAE and DOX, respectively. These results thus show release under a range of clinically-relevant doses.

### Cytotoxicity of Compounds of Formula (I) Before and After Cleavage

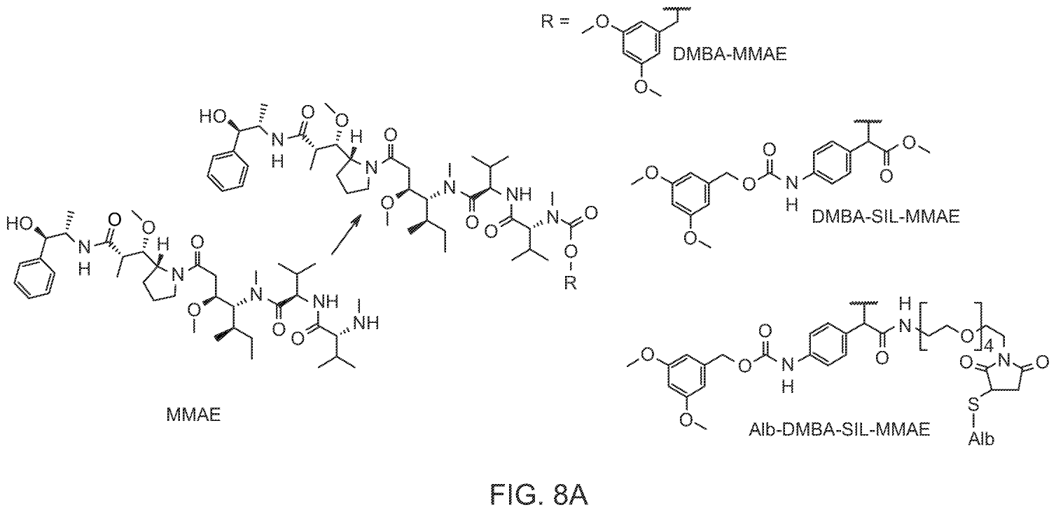

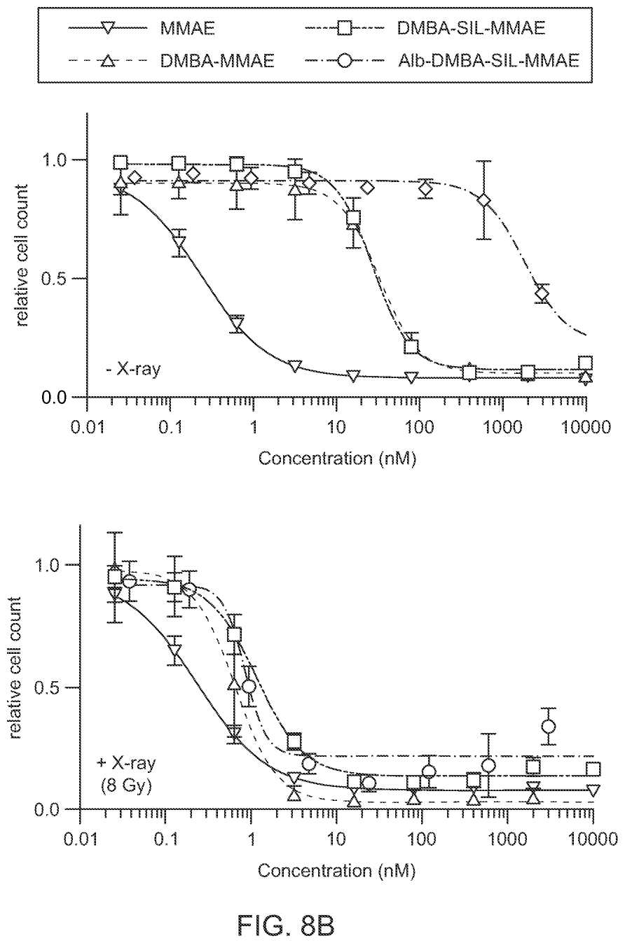

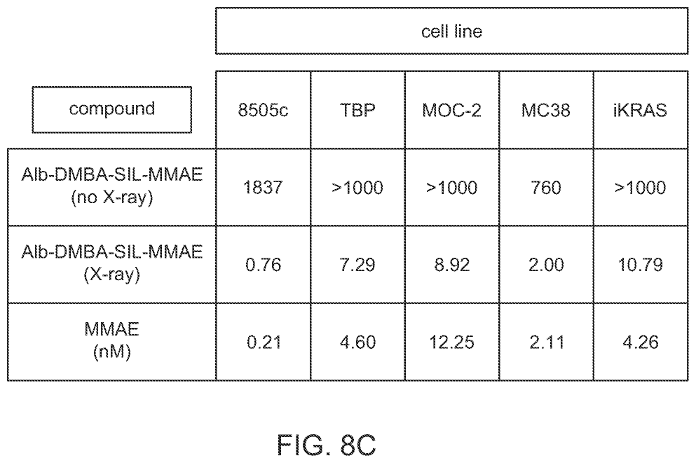

Experiments with cancer cells were performed to assess the degree to which the disclosed linker strategy shields biological activity of the drug payload under un-irradiated conditions, and restore drug activity once irradiated. Cancer cell lines were selected with constitutively active oncogenic signaling in the mitogen activated protein kinase (MAPK) RAS/RAF/MEK/ERK pathway, which has been previously shown as important for oncogene-driven uptake of serum albumin (Li, R. et al. Nat. Nanotechnol. 2021, 16, 830-839); cancer-types that are treated with cytotoxic agents and radiation therapy, including anaplastic thyroid cancer (ATC), a rare but aggressive malignancy that is associated with high degrees of resistance to traditional chemotherapy and radiation therapy were of particular focus. Drugs were irradiated prior to cancer cell treatment to allow assessment of drug action (caging and uncaging) independent of the biological effects of radiation to the cancer cells, which even at <10 Gy doses in cells considered radioresistant, such as human 8505c ATC cells, can substantially impact proliferation in vitro (Oweida, A. et al. Cancer Thyroid 2018, 28, 739-747). Compared to the parent MMAE drug, the drug-conjugate Alb-DMBA-SIL-MMAE exhibited 5,700-fold lower cytotoxicity in 8505c cells (FIG. 8). Anchoring the DMBA-SIL-MMAE to its protein vehicle, Alb, enhanced the caging of drug activity: Alb-DMBA-SIL-MMAE exhibited ˜40-fold lower cytotoxicity than the un-anchored compounds DMBA-SIL-MMAE and MMAE-DMBA (FIG. 8). In contrast, all three prodrugs exhibited similar cytotoxicity following 8 Gy irradiation, consistent with behavior of the parent drug and known efficiency of drug release at this dose (52±9%). Compared to prior DMBA caging approaches (Fu, Q. et al. Angew. Chem. Int. Ed. 2020, 59, 21546-21552), these data show that our linker strategy exhibits superior ability to limit drug activity under un-irradiated conditions, which is likely important for minimizing systemic toxicity. Furthermore, cytotoxicity of the parent drug is restored to a level commensurate with the fraction of released drug as measured by LC/MS, indicating that irradiation appropriately releases the payload in a fully active and intact form. Similar observations were found in cells derived from a genetically engineered mouse model of ATC (the TBP-3743 cell line) and other cancer cell lines (FIG. 8c and FIG. 20).

Given cytotoxicity measurements suggested parent MMAE activity was being restored following irradiation, it was subsequently assessed whether the molecular effects of MMAE were similarly affected. MMAE blocks the polymerization of tubulin into microtubules, which are critical cytoskeletal components that mediate mitotic cell division and metastatic invasion of cancer cells (Luthria, G. et al. Nat. Commun. 2020, 11, 3521; Dumontet, C. et al. Nat. Rev. Drug Discovery 2010, 9, 790-803). To directly visualize microtubule dynamics in live cancer cells, HT1080 EB3-mApple cell line was used, which transgenically expresses the fluorescent protein mApple fused to the protein EB3 (microtubule-associated protein RP/EB family member 3, MAPRE3). EB3 binds plus-end tips of growing microtubules, and time-lapse microscopy allows growing microtubules to be quantified for their abundance, growth velocities, and other features (Miller, M. A. et al. ACS nano 2018, 12, 12814-12826).

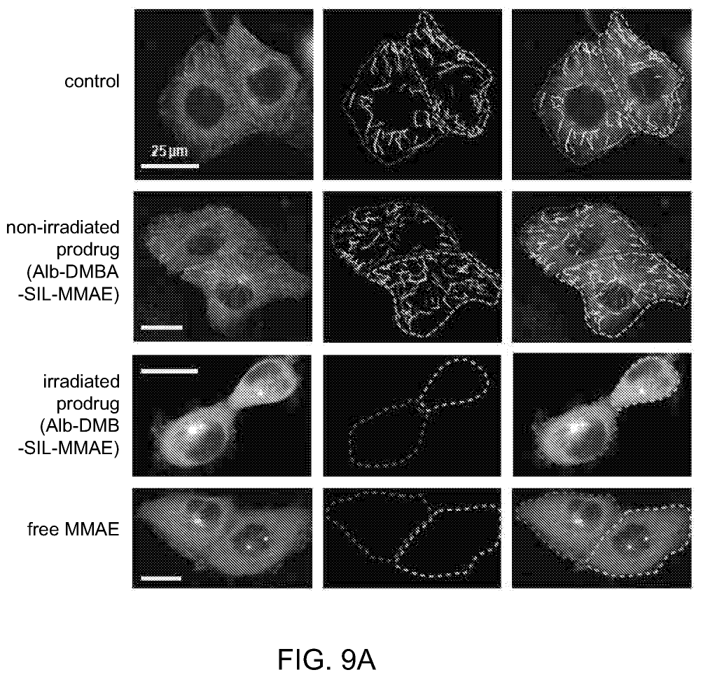

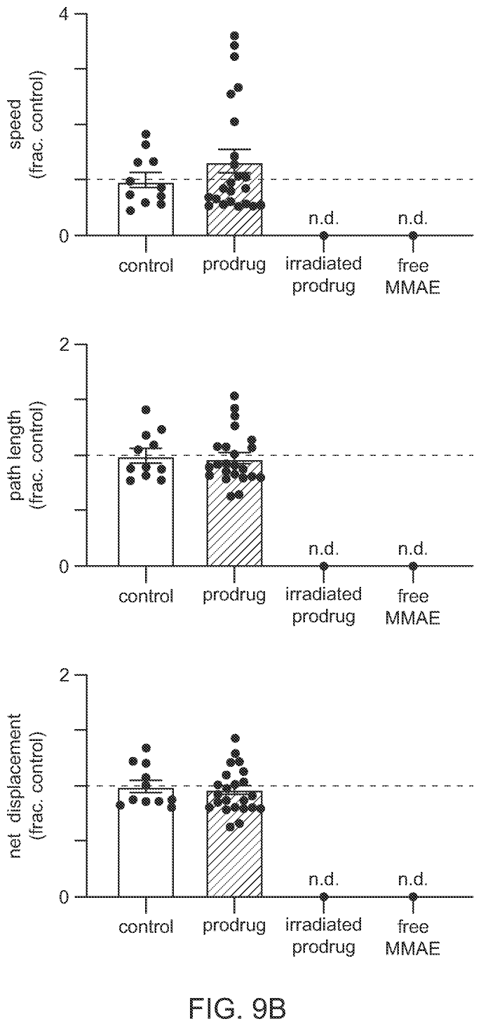

Using this approach, un-irradiated Alb-DMBA-SIL-MMAE elicited no significant impacts on cancer cell microtubule dynamics, in contrast, dynamics were totally eliminated with irradiated drug (FIG. 9). These results confirm that the linker strategy efficiently cages drug activity, and show that the activity of MMAE to disrupt microtubule dynamics is restored upon radiation-mediated drug release.

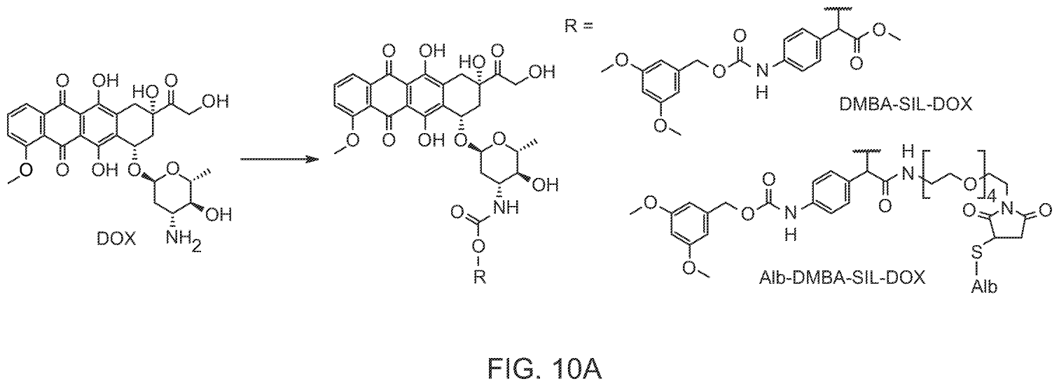

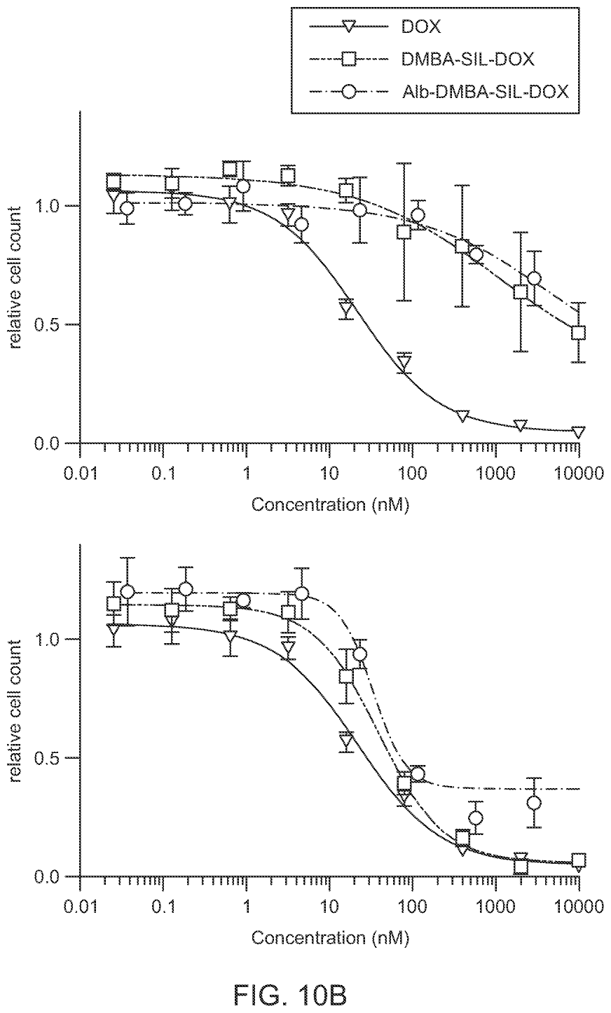

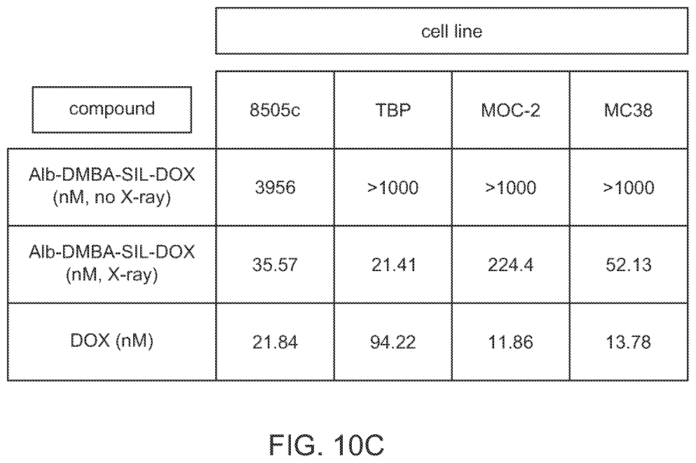

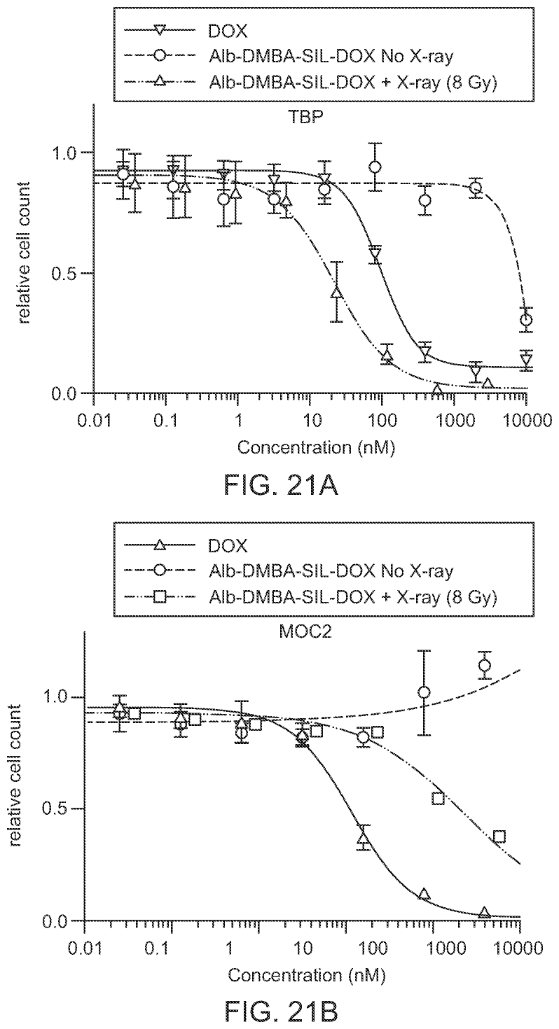

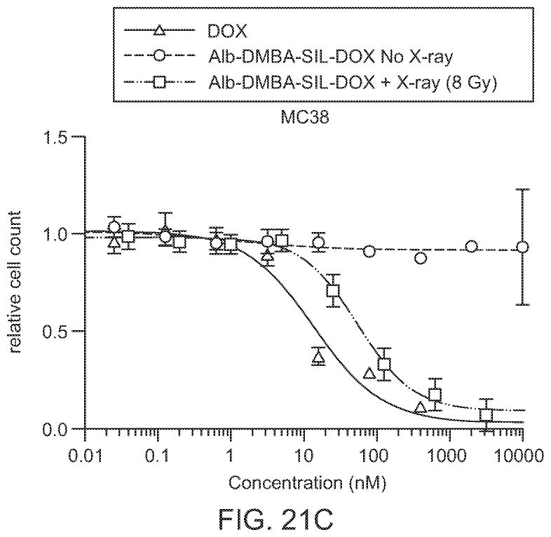

Similar experiments were performed to test the caging and activation efficiencies of Alb-DMBA-SIL-DOX, compared to the parent compound doxorubicin (FIG. 10). The concentration at which drug inhibited 50% of ATC cell growth was >10 μM for un-irradiated Alb-DMBA-SIL-DOX and DMBA-SIL-DOX. This was roughly 100-fold higher than that observed for the parent drug (IC₅₀=94 nM). Irradiation restored prodrug cytotoxicity to a level to be expected based on the fraction of drug released (0.5±0.1). Similar results were observed in other cancer cell lines (FIG. 21). Compared to MMAE, the DMBA-SIL-DOX did not benefit further from conjugation to serum albumin, in terms of limiting the activity of un-irradiated compound. This is potentially due to the distinct DOX mechanism of action compared to MMAE (FIG. 8B).

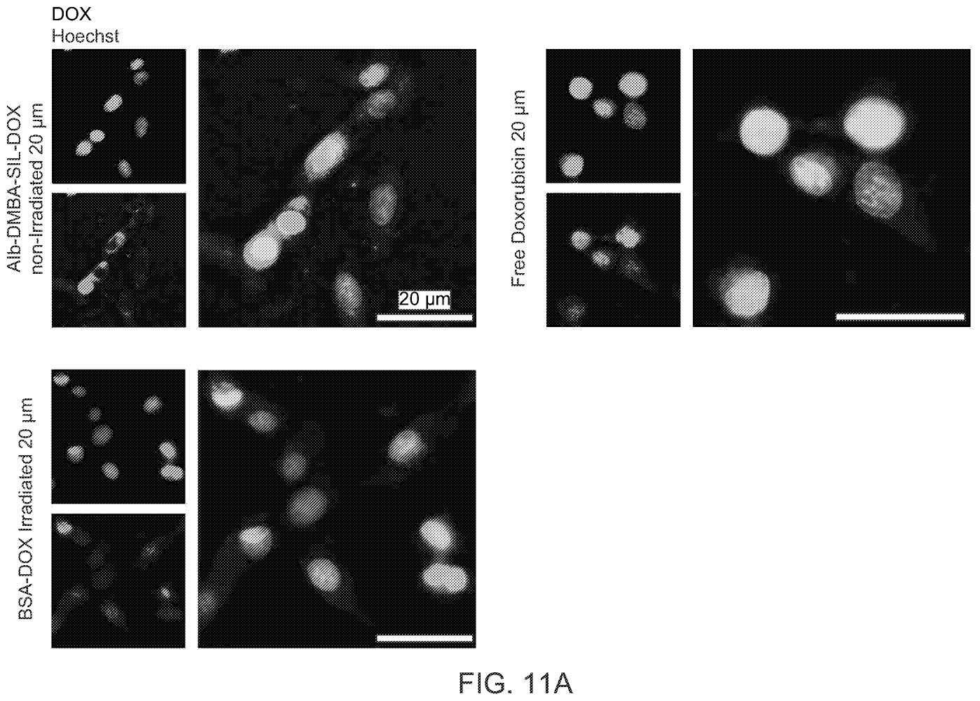

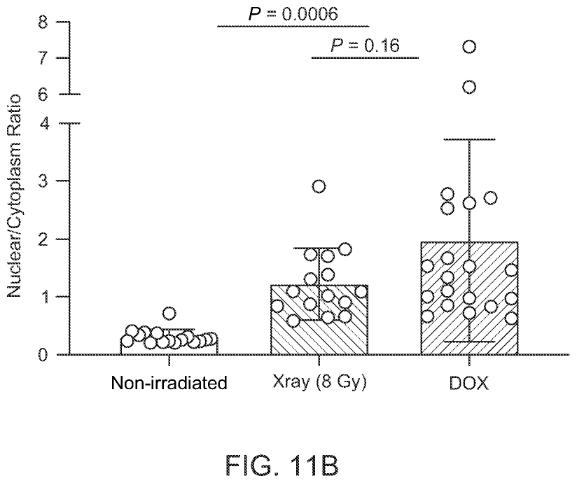

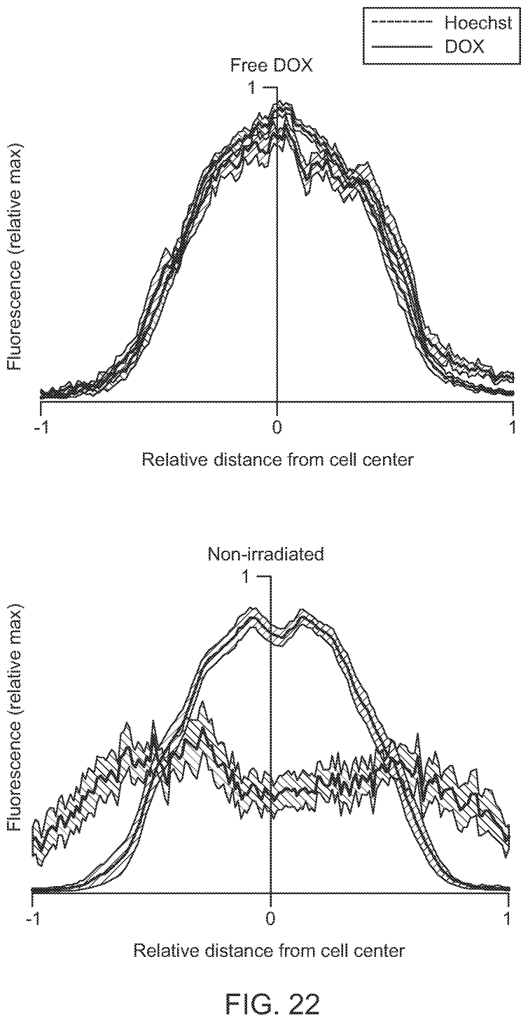

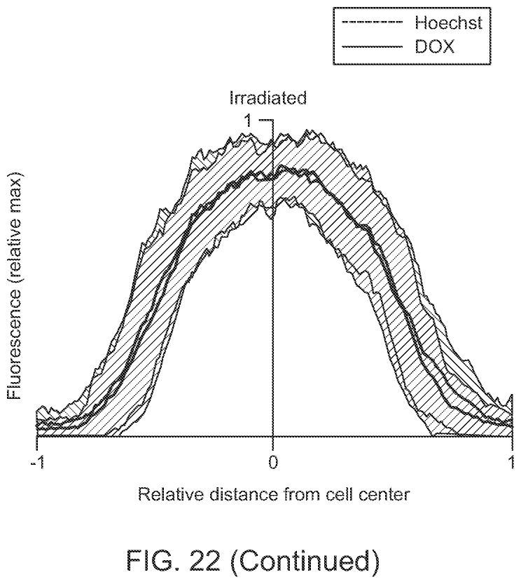

Doxorubicin and prodrug derivatives exhibit intrinsic fluorescence that is visible by confocal microscopy, and imaging can therefore be used to assess drug accumulation and co-localization with its target in the nuclei of live cancer cells (Miller et al. Nat. Common. 2017, 8, 15906). Doxorubicin is an anthracycline that intercalates DNA, inhibits topoisomerase II, and therefore generates DNA damage leading to cell death. The 3′ amino of the daunosamine moiety on doxorubicin forms a covalent bond with the exocyclic amino of guanine, and this site is frequently modified to cage drug activity (Miller, M. A. et al. ACS nano 2018, 12, 12814-12826), as done here as well. ATC cells show nuclear accumulation of doxorubicin and irradiated Alb-DMBA-SIL-DOX (FIG. 11 and FIG. 22). In contrast, unirradiated prodrug is confined to the cytoplasm, therefore suggesting the unirradiated drug-conjugate remains intact, such that doxorubicin is unable to freely enter the nucleus and interact with DNA. Overall, these results indicate that the radiation-cleavable linker blocks the ability of doxorubicin to intercalate DNA and elicit cytotoxic effects, and that drug irradiation can appropriately release it to facilitate nuclear localization and cytotoxicity.

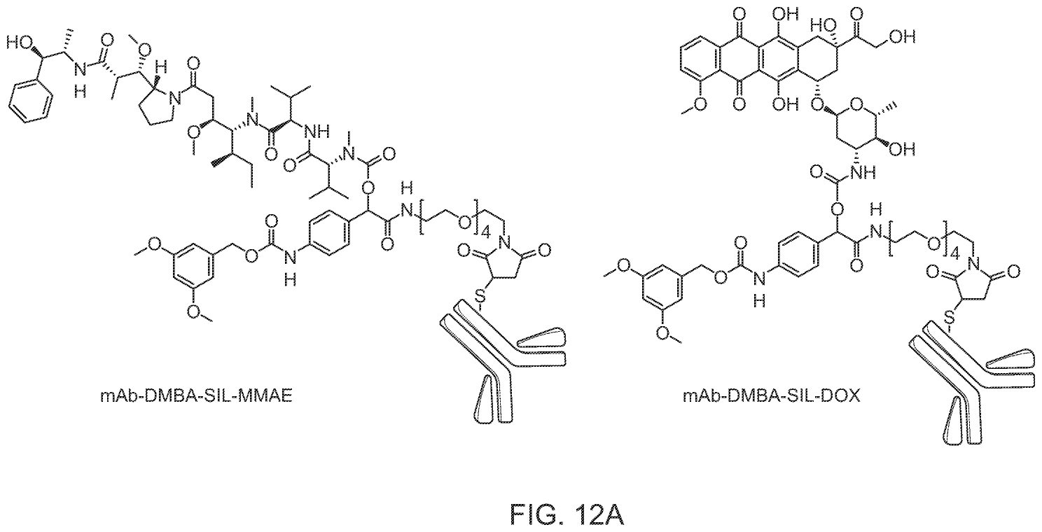

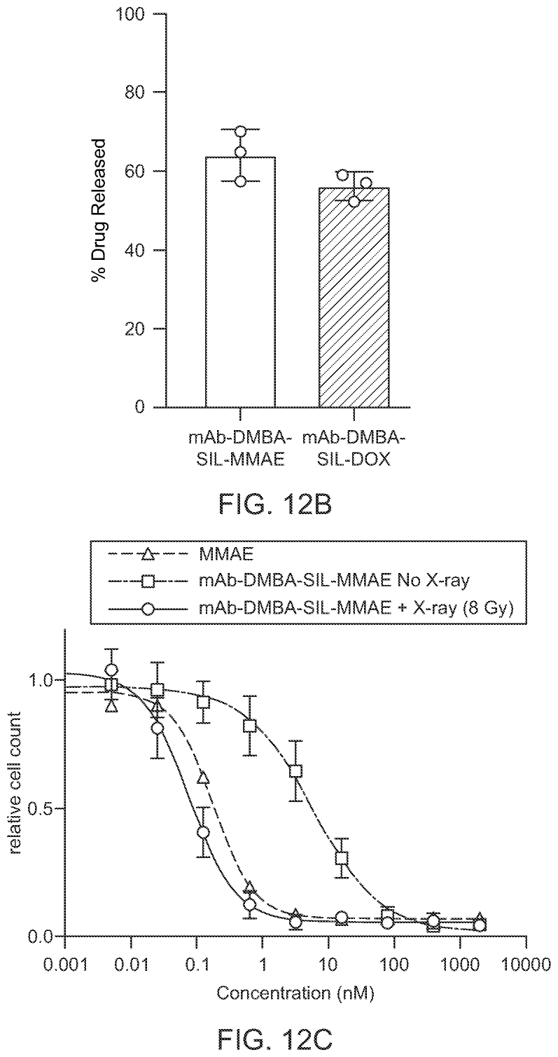

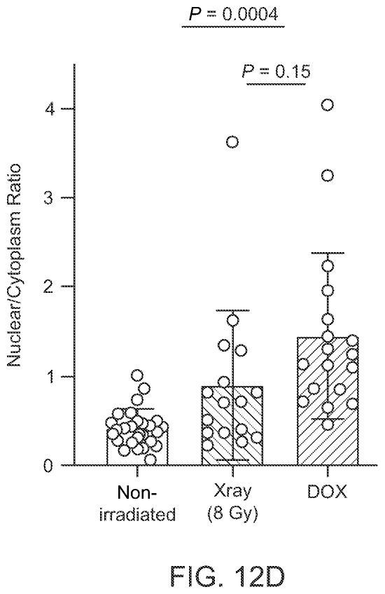

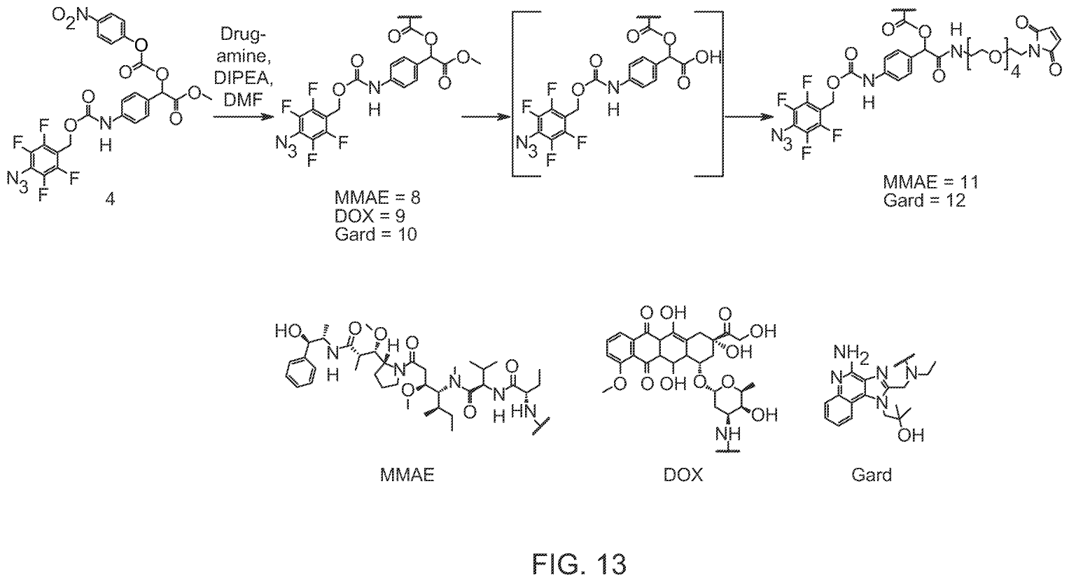

To examine the generalizability of the linker strategy beyond albumin-conjugates, antibody drug conjugate (ADC) with a radiation-cleavable cytotoxic payload were also prepared and tested. ADCs of the DMBA-SIL-DOX/MMAE prodrugs were prepared by conjugating them to a model tumor-targeted monoclonal antibody that binds epidermal growth factor receptor (αEGFR mAb). Multiple αEGFR-mAb are used clinically including cetuximab and panitumumab for a variety of solid tumors, and @EGFR-ADC are under development. The results of cleavage experiments showed that antibody-conjugates performed similarly to albumin-conjugates, releasing with radiation 64±7% and 56±4% of the estimated MMAE and DOX, respectively (FIG. 12A). In a cytotoxicity assay using the 8505c ATC cell line, the mAb-DMBA-SIL-MMAE conjugate demonstrated a 70-fold increase in cytotoxicity after X-ray irradiation (8 Gy) compared to the non-irradiated conjugate (FIG. 12B).

Similarly, irradiated mAb-DMBA-SIL-Dox demonstrated an increased nuclear to cytoplasm ratio in the sub-cellular distribution of the doxorubicin payload relative to its non-irradiated counterpart (FIG. 12C). Taken together, these data show that mAb conjugation is effective in blocking payload activity, and that radiation exposure releases payload from the mAb-conjugate and unleashes payload activity in the cancer cells.

### Chemical Prodrug Activation in Live Cells and In Vivo

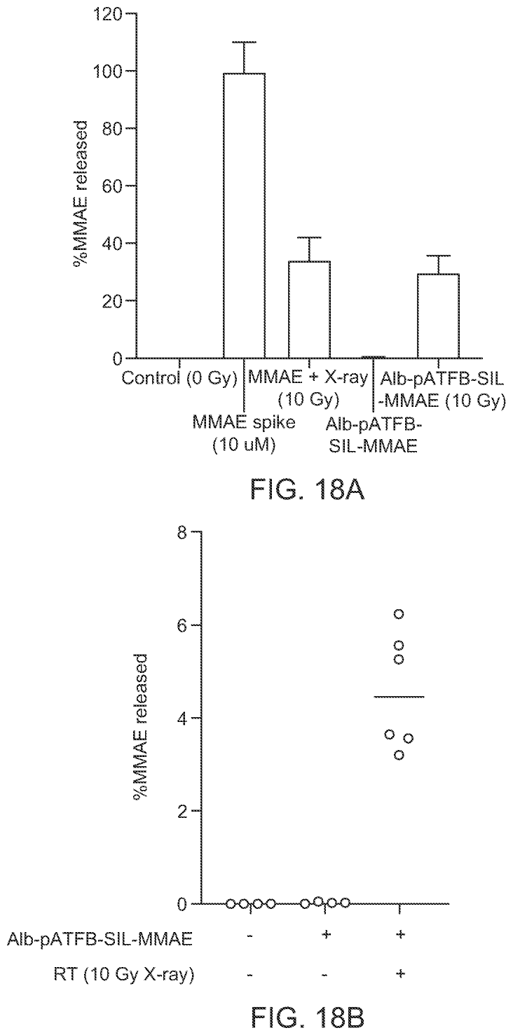

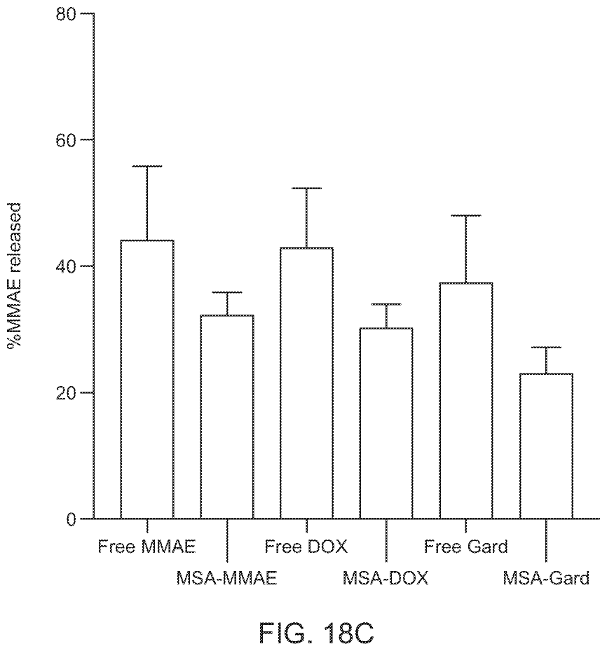

pATFB-caged compounds are chemically activatable in live cell cultures. Cancer cells treated with Alb-pATFB-SIL-MMAE accumulate the albumin vehicle, and subsequent 10 Gy irradiation activates roughly 50% of the drug (FIG. 18A). In a syngeneic mouse model of cancer, C57Bl/6-background mice bearing subcutaneous MC38 allografts show accumulation of chemically activated MMAE payload in irradiated but not non-irradiated tumors, even in non-irradiated contralateral tumors of the same mouse (FIG. 18B). Alb-pATFB-SIL-MMAE prodrug was given intravenously 24 hours prior to 10 Gy irradiation locally at the tumor site. Irradiated and non-irradiated bilateral tumors were then excised after 10 minutes and examined by LC/MS for accumulation of activated uncaged MMAE (FIG. 18B). This data indicates that Alb-pATFB-SIL-MMAE accumulates in tumor tissues and is locally activated by external beam irradiation to yield local, clinically relevant concentrations of the activated drug.

The ability to activate pATFB drugs in live cells is not limited to Alb-pATFB-SIL-MMAE. Drug activation was detected for other pATFB-containing DOX and Gard compounds (FIG. 18C).

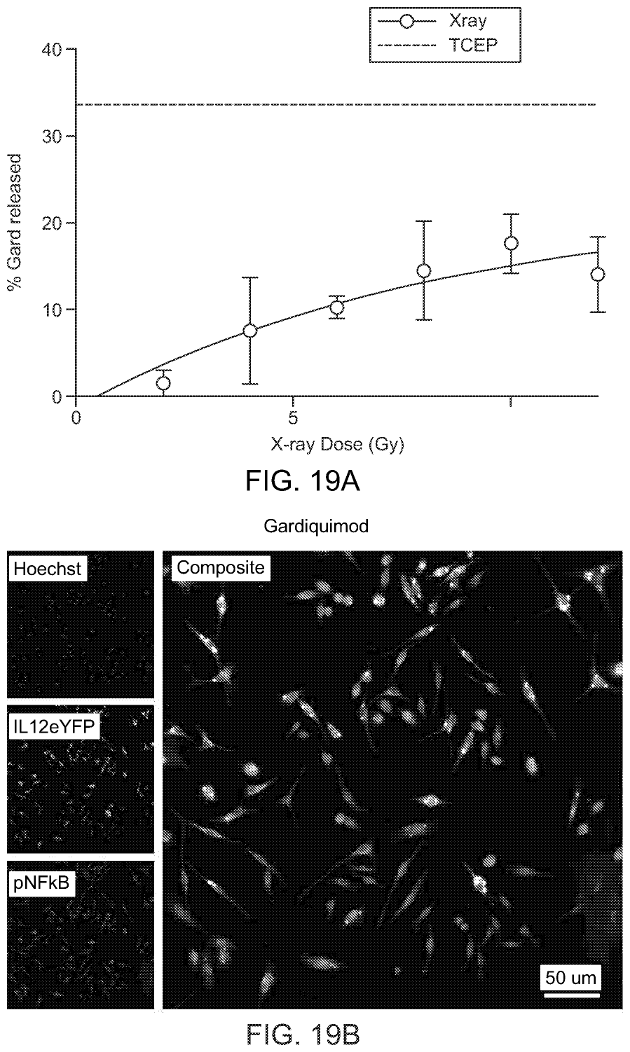

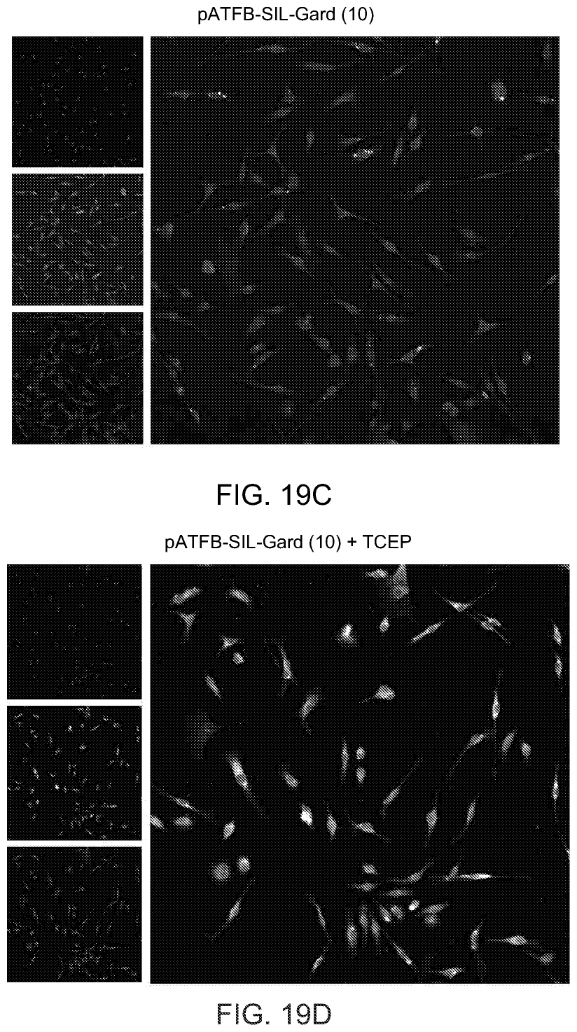

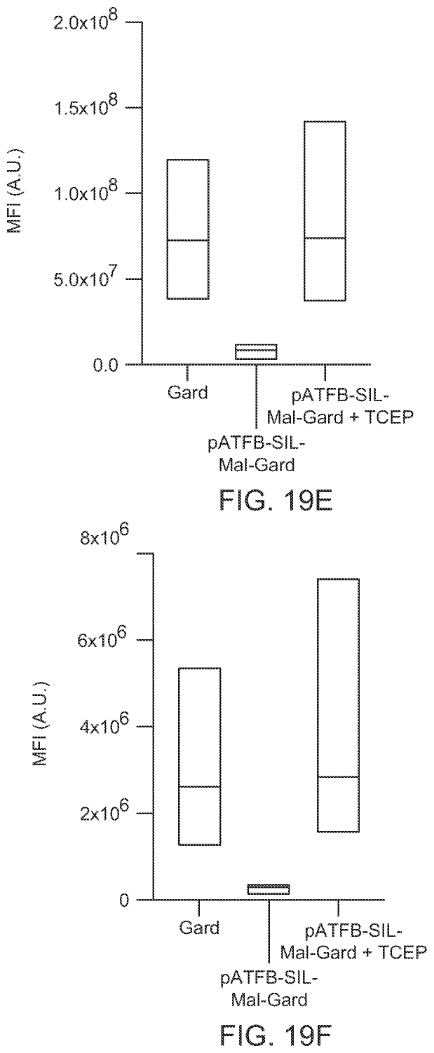

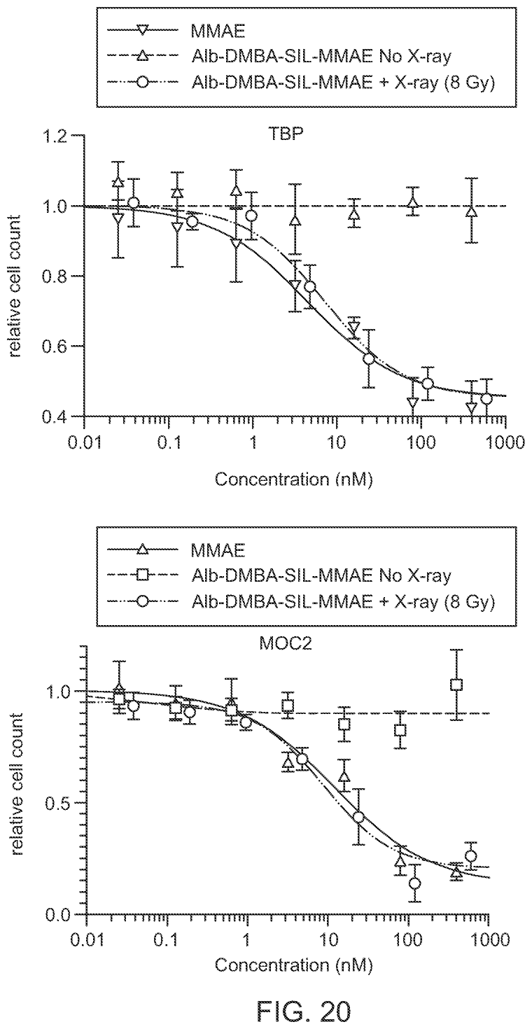

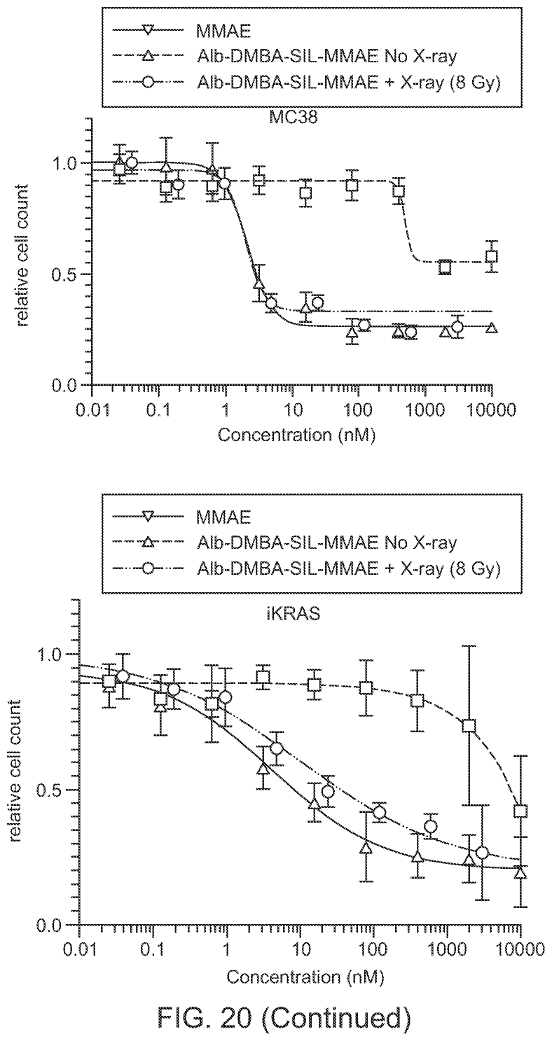

Caged agonist of toll like receptor 7 (TLR7a) based on gardiquimod also showed chemical release following irradiation comparable to the other pATFB-caged compounds (FIG. 19A). The pATFB-SIL-Gard caged TLR7a exhibits attenuated immunostimulatory behavior compared to free gardiquimod, and chemical uncaging restores the immunostimulatory activity (FIG. 19B-F). These data indicate that radiation can chemically release TLR7a, and such chemical cleavage enhances TLR7a biological activity.

In conclusion, this work extends radiation-activated chemistry to support triggered release of biologic drug-conjugates. As with traditional ADC linkers, the approach can be applied to diverse delivery vehicles such as serum albumin or antibodies as we demonstrate here; it furthermore can be applied to diverse drug payloads, including the widely used cytotoxic chemotherapies doxorubicin and MMAE. We show that the linker cleavage can be triggered by multiple forms of ionizing radiation and is accelerated under hypoxic conditions thought to stimulate macropinocytosis of albumin-bound agents (Zhang, M. S. et al. Nat. Commun. 2022, 13, 954). Radiation itself has been shown to improve drug penetration into tumors (Miller, M. A. et al. Sci. Transl. Med 2017, 9, eaa10225; Miller, M. A. et al. ACS nano 2018, 12, 12814-12826; and Kim, H.-Y. et al. ACS Nano 2018, 12, 12015-12029) and therefore offers the possibility of self-amplifying local payload activity in this case. This work sets the stage for future work to investigate mechanisms of in vivo drug activation and potential off-target payload release, which will require i) careful analysis of chemical and biological drug behaviors in tumors and off-target tissue, with and without radiation; ii) comparison with free (non-conjugated) drugs and prodrugs, as well as non-cleavable drug-conjugates; and iii) assessment of drug behavior in orthotopic, patient-derived, and/or autochthonous tumor models that recapitulate the hypoxic tumor microenvironments often found in patients (Ng, T. S. C. et al. Sci. Adv. 2022, 8, eab16339). This work also establishes a foundation from which to assess drug-conjugate payloads, beyond MMAE and doxorubicin, that may synergistically combine with the biological effects of radiation. Although the therapeutic radiation doses used here are known to elicit strong impacts on cancer cell behavior in vitro—for instance by reducing proliferation of the cancer cells used in this work (8505c, MC38, HT1080) by >95% in clonogenic assays—such doses nonetheless fail to eradicate tumors and block disease progression (Miller, M. A. et al. Sci. Transl. Med. 2017, 9, eaa10225; Oweida, A. et al. Thyroid 2018, 28, 739-747; and Jones, K. I et al. EMBO. Mol. Med. 2018, 10, e9342). To this end, the modular design strategy lends itself to further optimization of the X-ray activated trigger, the conjugation anchor, and the drug payload.

The presently disclosed method of prodrug engineering may be applied to a variety of therapeutics including immunomodulatory agents and targeted inhibitors that may be chosen to synergistically combine with radiation administered as part of standard of care. Extensive research has already gone into understanding how DNA- and microtubule-targeted drugs may best combine with radiation therapy, with special focus on optimizing cancer cells to be in the radiosensitive G2/M phase of their cell cycle, and on tumor microenvironment effects that maximize oxygenation. In contrast, the linker approach disclosed herein offers a distinct way of considering synergistic effects under conditions of hypoxia, and local drug delivery may allow higher local drug concentrations or more potent drug payloads to be considered.

### Examples

### Materials and Methods

### Preparation of Compounds

Preparation of 3,5-dimethoxybenzyl alcohol (DMBA) prodrugs containing monomethyl auristatin E (MMAE) and doxorubicin (DOX) is shown in Scheme 1.

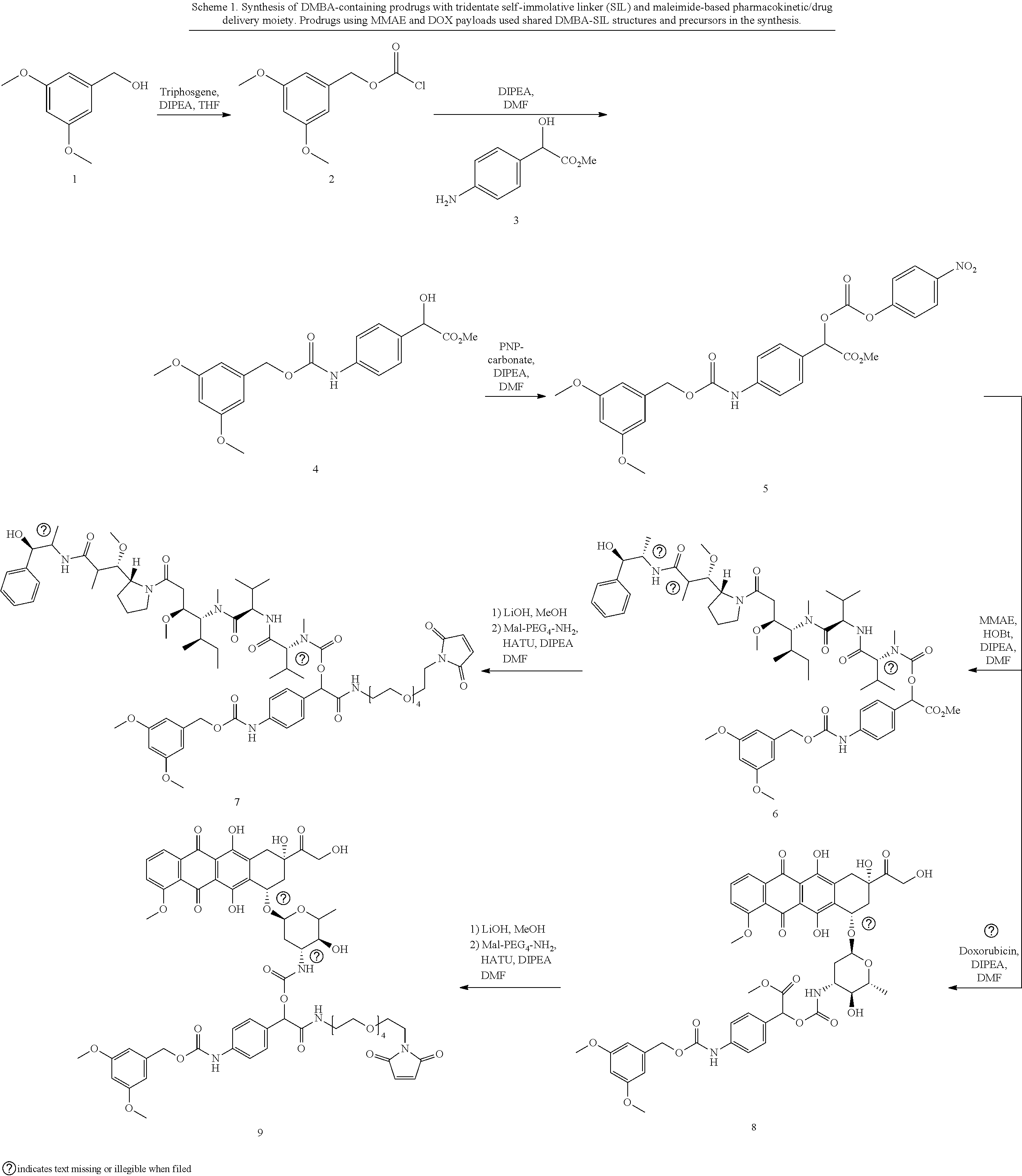

The overall synthesis of the Alb-DMBA-SIL-MMAE or Alb-DMBA-SIL-DOX were performed in 5 steps (Scheme 1). Self-immolative linker 3 was synthesized as previously described (WO 2015/038426, PCT/US2014/054236, Mar. 19, 2015.) and installation of the X-ray activated trigger was achieved by first reacting benzyl alcohol 1 with triphosgene to produce the chloroformate 2, which was then reacted directly with aniline 3 to yield DMBA-SIL 4. The benzylic alcohol on this molecule was activated by reaction with bis(4-nitrophenyl) carbonate under basic conditions with diisopropyl ethyl amine to generate carbonate 5. This carbonate could then be reacted with either MMAE or DOX to yield compounds 6 and 8, respectively. Finally, maleimide was installed via hydrolysis followed by amide coupling to obtain products 7 and 9. Prodrugs containing the radiation-cleavable pATFB moiety were prepared similarly, with full details disclosed in the compound preparation section herein.

Preparation of comparative prodrugs not containing the SIL component are shown in FIG. 4.

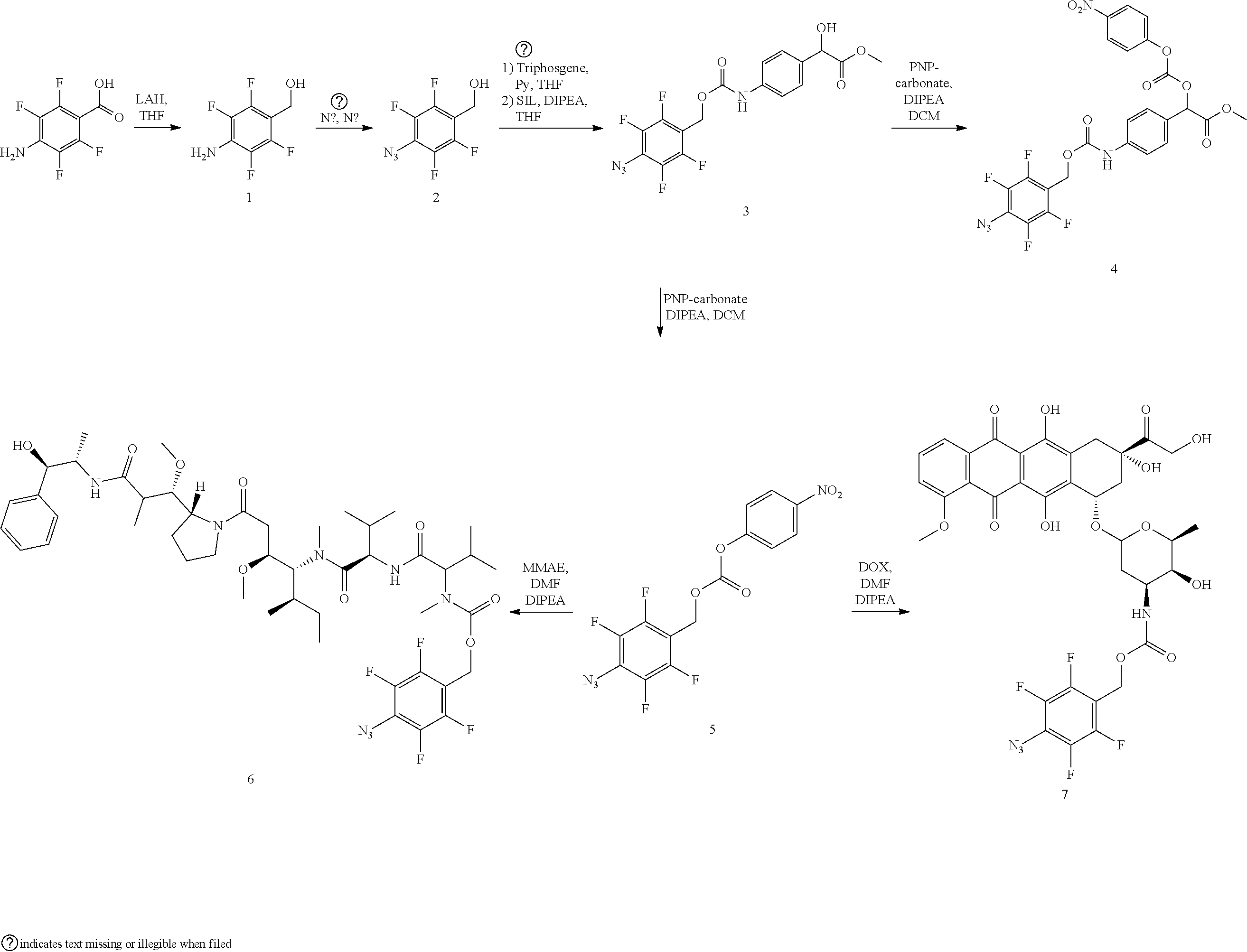

Compounds 6 and 7 were prepared following the steps previously described for compound 7 (Geng J, et al. Nat Chem. 2021 August; 13 (8): 805-810), with full details disclosed in the compound preparation section herein.

### Conjugation to Carriers

The maleimide-containing prodrugs were conjugated to Alb by incubation at room temperature in PBS (pH 7.4, Gibco) for two hours, followed by spin filtration (30 kDa MWCO) to remove unreacted prodrug, and further purification by size exclusion chromatography. For conjugation to an antibody, e.g., a model tumor-targeted monoclonal antibody that binds epidermal growth factor receptor (@EGFR mAb), antibody disulfide bonds were first with tris(2-carboxyethyl) phosphine hydrogen chloride (TCEP-HCl), followed by conjugation with the respective prodrugs via thiol-maleimide Michael addition. The resulting mAb-DMBA-SIL-DOX and mAb-DMBA-SIL-MMAE were then subjected to the same in vitro analyses as the albumin conjugates to demonstrate X-ray activation and determine drug release efficiencies.

### General Information

Unless stated otherwise, all materials were used as received. Trimethylsilyl cyanide (TMS-CN), 4-nitrobenzaldehyde, glacial acetic acid (AcOH), sulfuric acid, palladium on carbon (10 wt. %), zinc iodide, triphosgene, N,N-diisopropylethylamine, tetrahydrofuran, N,N-dimethylformamide, bis(4-nitrophenyl) carbonate, lithium hydroxide, and HATU were purchased from Sigma Aldrich (St. Louis, MO, USA). Hydrochloric acid (HCl), methanol, dichloromethane, and acetonitrile were purchased from VWR International (Radnor, PA, USA), while monomethyl auristatin E and doxorubicin hydrochloride were purchased from MedChem Express (Monmouth Junction, NJ, USA). Maleimide-PEG4-amine trifluoroacetic acid salt was purchased from BroadPharm (San Diego, CA, USA) and 3,5-dimethoxybenzyl alcohol was purchased from Fisher Scientific (Hampton, NH, USA). DMSO-d6, MeOD-d4, and CDCl3 were purchased from Cambridge Isotope Laboratories (Tewksbury, MA, USA). Reaction mixtures were purified using a Biotage SNAP Bio C18 300 A 10 g on a Biotage Isolera with a gradient composed of water (0.1% formic acid) and acetonitrile (0.1% formic acid) for reversed-phase chromatography. 1H and 13C NMR spectra were recorded on a Bruker AC-400 MHz spectrometer. High performance liquid chromatography-mass spectrometry analysis (HPLC-MS, LCMS) was performed on a Waters instrument equipped with a Waters 2424 ELS Detector, Waters 2998 UV-Vis Diode array Detector, Waters 2475 Multi-wavelength Fluorescence Detector, and a Waters 3100 Mass Detector. Separations employed an HPLC-grade water/acetonitrile solvent gradient. Columns: XTerra MS C18 Column, 125., 5 μm, 4.6 mm×50 mm column. HRMS analysis was carried out on a Thermo Scientific Dionex UltiMate 3000 UHPLC coupled to a Thermo Q Exactive Plus mass spectrometer system (Thermo Fisher Scientific Inc, Waltham, MA) equipped with an HESI-II electrospray ionization (ESI) source. Data were acquired with Chromeleon Xpress software for UHPLC and Thermo Xcalibur software version 3.0.63 for mass spectrometry, and processed with Thermo Xcalibur Qual Browser software version 4.0.27.19.

### Preparation of methyl 2-(4-aminophenyl)-2-hydroxyacetate

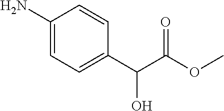

### Step 1: Preparation of 2-Hydroxy-2-(4-nitrophenyl) acetic acid

To a solution of 4-nitrobenzaldehyde (5 g, 33 mmol) in dichloromethane (50 mL) was added zinc iodide (1.05 g, 3.3 mmol) and trimethylsilyl cyanide (TMS-CN) (4.56 g, 46 mmol). The mixture was then heated to reflux for 4 hours before 1M HCl (30 mL) was added and heating continued at 60° C. for 14 hours. The mixture was then cooled to room temperature, diluted with water (50 mL) and extracted with dichloromethane (3×100 mL). The combined organics were then washed with water (200 mL) and brine (200 mL), dried over magnesium sulfate and concentrated. The crude intermediate was then dissolved in acetic acid (40 mL) and 10M HCl (40 mL) was added. The mixture was heated to 100° C. for 16 hours, then cooled to room temperature, concentrated and dried overnight under vacuum to provide the crude product as an orange solid, which was used without further purification.

### Step 2. Preparation of methyl 2-hydroxy-2-(4-nitrophenyl)acetate

The crude 2-Hydroxy-2-(4-nitrophenyl) acetic acid from step 1 was dissolved in methanol (50 mL) and concentrated sulfuric acid (4 mL) was added. The solution was refluxed for 14 hours, then cooled to room temperature and concentrated under reduced pressure. The residue was dissolved in EtOAc (150 mL), washed with water (100 mL) and brine (100 mL), and dried over magnesium sulfate. The solvent was evaporated and the residue was purified by column chromatography (0-50% EtOAc in hexanes) to obtain S3 as a yellow solid (4.19 g, 60% yield). TH NMR (400 MHz, CDCl3): δ 8.17 (d, J=8.8 Hz, 2H), 7.62 (d, J=8.8 Hz, 2H), 5.30 (s, 1H), 3.91 (brs, 1H), 3.75 (s, 3H). ¹³C NMR (101 MHz, CDCl3): δ 172.9, 147.9, 145.1, 127.5, 123.7, 72.1, 53.5.

### Step 3: Preparation of methyl 2-(4-aminophenyl)-2-hydroxyacetate

The crude methyl 2-hydroxy-2-(4-nitrophenyl)acetate obtained from step 2 was dissolved in methanol (50 mL) and the reaction vessel was flushed with argon. Palladium on activated charcoal (10% Pd/C, 1 g) was added to the solution and the mixture was then flushed with H₂ and stirred under positive pressure (balloon) for 24 hours. The mixture was then filtered over Celite and concentrated under reduced pressure to obtain compound 3 (3.52 g, 98% yield). ¹H NMR (400 MHz, MeOD-d4): δ 7.14 (d, J=8.4 Hz, 2H), 6.69 (d, J=8.4 Hz, 2H), 5.03 (s, 1H), 3.68 (s, 3H). ¹³C NMR (101 MHz, CDCl3): δ 175.5, 149.3, 129.5, 129.0, 118.2, 74.2, 52.5.

### Preparation of MMAE-DMBA

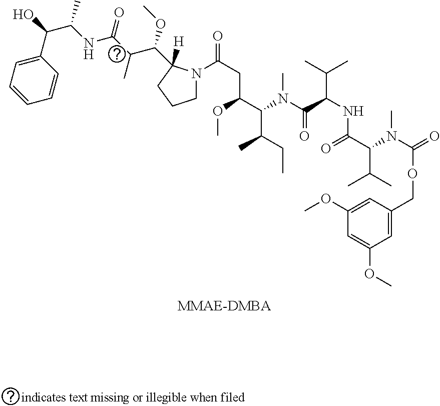 [SMILES: CC[C@@H](C)[C@H]([C@H](CC(=O)N1CCC[C@@H]1[C@@H](OC)C(C)C(=O)N[C@@H](C)[C@H](O)c1ccccc1)OC)N(C)C(=O)[C@H](NC(=O)[C@@H](C(C)C)N(C)C(=O)OCc1cc(OC)cc(OC)c1)C(C)C]

### Step 1. Preparation of 3,5-dimethoxybenzyl (4-nitrophenyl) carbonate

Bis(4-nitrophenyl) carbonate (668 mg, 2.19 mmol) was added to a solution of 3,5-dimethoxybenzyl alcohol (DMBA) (378 mg, 2.25 mmol). DIPEA (400 μL, 2.30 mmol) was added dropwise and the mixture was left to shake for 2.5 hours at room temperature. The mixture was then loaded directly onto a reverse-phase column and purified using a gradient of 5-100% acetonitrile (0.1% formic acid) in water (0.1% formic acid). The product was isolated as a white solid (507.4 mg, 68% yield). ¹H NMR (400 MHz, CDCl3): δ 8.25 (d, J=9.2 Hz, 2H), 7.37 (d, J=9.2 Hz, 2H), 6.56 (d, J=2.4 Hz, 2H), 6.46 (t, J=2.3 Hz, 1H), 5.22 (s, 2H), 3.80 (s, 6H). ¹³C NMR (101 MHz, CDCl3): δ 161.1, 155.6, 152.5, 145.4, 136.4, 125.4, 121.8, 106.3, 100.8, 70.9, 55.5.

### Step 2. Preparation of MMAE-DMBA

MMAE (10.4 mg, 14.5 μmol) and 1-Hydroxybenzotriazole hydrate (5.0 mg, 37 μmol) were dissolved in dry DMF (1 mL) and 3,5-dimethoxybenzyl (4-nitrophenyl) carbonate (DMBA-PNP) (12 mg, 36.0 μmol) was added. Once everything had dissolved, DIPEA (10 μL, 57.4 μmol) was added and the reaction was stirred at 25° C. for 20 hours. The reaction mixture was then loaded directly onto a reverse phase column and purified using a gradient of 5-100% acetonitrile in water (0.1% formic acid). The fractions were then evaporated to obtain the product as a white solid (6.1 mg, 46% yield). ¹H NMR (400 MHz, CDCl3): δ 7.39-7.31 (m, 3H), 6.60-6.49 (m, 3H), 6.42-6.38 (m, 1H), 5.11 (s, 1H), 4.72-4.66 (m, 1H), 4.15 (s, 1H), 4.06 (s, 1H), 3.93 (s, 1H), 3.88 (s, 1H), 3.42 (s, 2H), 3.39 (s, 1H), 3.31 (s, 2H), 3.01 (s, 1H), 2.92 (s, 2H), 2.50-2.41 (m, 1H), 2.28-2.18 (m, 1H), 1.68 (s, 1H), 1.43-1.32 (m, 1H), 1.26 (d, J=7.0 Hz, 2H), 1.05-0.80 (m, 17H). ¹³C NMR (101 MHz, CDCl3): δ 174.8, 161.1, 128.2, 127.4, 126.5, 106.

0, 105.6, 100.7. 82.1, 69.8, 65.2, 60.3, 58.1, 55.5, 51.8, 48.0, 33.6, 29.7, 25.9, 25.2, 19.5, 18.7, 14.0. MS: m/z: calculated for C49H78N5O11 [M+H]+ 912.57 found 913.02.

### Preparation of methyl 2-(4-((((3,5-dimethoxybenzyl)oxy) carbonyl)amino)phenyl)-2-(((4-nitrophenoxy) carbonyl)oxy)acetate (DMBA-SIL-PNP)

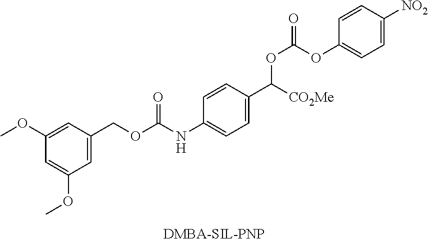 [SMILES: COC(=O)C(OC(=O)Oc1ccc([N+](=O)[O-])cc1)c1ccc(NC(=O)OCc2cc(OC)cc(OC)c2)cc1]

### Step 1: Preparation of 3,5-dimethoxybenzyl carbonochloridate

3,5-dimethoxybenzyl alcohol (DMBA) (0.818 g, 4.87 mmol) was dissolved in anhydrous THF (20 mL) and triphosgene (481.7 mg, 1.623 mmol) was added while stirring. DIPEA (900 μL, 5.17 mmol) was then added and the reaction left to stir at room temperature for 2 hours. The mixture was then filtered to remove the resulting precipitate, then evaporated, and the resulting residue was used without further purification.

### Step 2: Preparation of methyl 2-(4-((((3,5-dimethoxybenzyl)oxy)carbonyl)amino)phenyl)-2-hydroxyacetate (DMBA-SIL)

methyl 2-(4-aminophenyl)-2-hydroxyacetate (102 mg, 564 μmol) and DIPEA (100 μL, 574 μmol) were added to a solution of 3,5-dimethoxybenzyl carbonochloridate (from step 1) in THF (2 mL) and the reaction left on a shaker at 25° C. for 16 hours. The solvent was then removed by rotary evaporation and the resulting residue was dissolved in DMSO (2 mL) and loaded directly onto a reverse phase column. The product was isolated using a gradient of 5-100% acetonitrile (0.1% formic acid) in water (0.1% formic acid) as a white solid (56 mg, 32% yield over 2 steps). ¹H NMR (400 MHz, CDCl3): δ 7.38 (d, J=8.7 Hz, 2H), 7.32 (d, J=8.7 Hz, 2H), 6.97 (s, 1), 6.52 (d, J=3.3 Hz, 2H), 6.41 (t, J=2.3 Hz, 1H), 5.13 (s, 1H), 5.11 (s, 2H), 3.77 (s, 6H), 3.73 (s, 3H), 3.56 (brs, 1H). ¹³C NMR (101 MHz, CDCl3): δ 174.2, 161.1, 153.4, 138.3, 138.2, 133.4, 127.5, 118.9, 106.1, 100.3, 72.55, 67.1, 55.5, 53.11. MS: m/z: calculated for C19H20NO7 [M−H]− 374.12 found 374.24.

### Step 3. Preparation of DMBA-SIL-PNP

Bis(4-nitrophenyl) carbonate (105 mg, 345 μmol) was added to a solution of methyl 2-(4-((((3,5-dimethoxybenzyl)oxy) carbonyl)amino)phenyl)-2-hydroxyacetate (DMBA-SIL) (65.8 mg, 175 μmol) and DIPEA (50 μL, 287 μmol) in dry DMF (2 mL) at 0° C. This mixture was allowed to warm to room temperature and stirred for 4 hours, then added to a 1:1 mixture of water: ethyl acetate. The ethyl acetate layer was collected and the aqueous phase was extracted twice with ethyl acetate. The combined organics were dried over magnesium sulfate followed by rotary evaporation. The resulting residue was dissolved in DMSO (1 mL), loaded onto a reverse-phase column and purified using a gradient of 5-100% acetonitrile (0.1% formic acid) in water (0.1% formic acid). The product was acquired as a white solid (42.3 mg, 51% yield). ¹H NMR (400 MHz, CDCl3): δ 8.28 (d, J=9.2 Hz, 2H), 7.46-7.40 (m, 6H), 6.81 (s, 1H), 6.54 (d, J=2.3 Hz, 2H), 6.43 (t, J=2.3 Hz, 1H), 5.92 (s, 1H), 5.14 (s, 2H), 3.80 (s, 6H), 3.78 (s, 3H). ¹³C NMR (101 MHz, CDCl3): δ 168.6, 161.2, 155.4, 153.1, 152.0, 145.7, 139.4, 138.1, 128.9, 127.4, 125.5, 121.9, 119.0, 106.2, 100.4, 77.9, 67.3, 55.5, 53.2.

### Preparation of DMBA-SIL-MMAE (6)

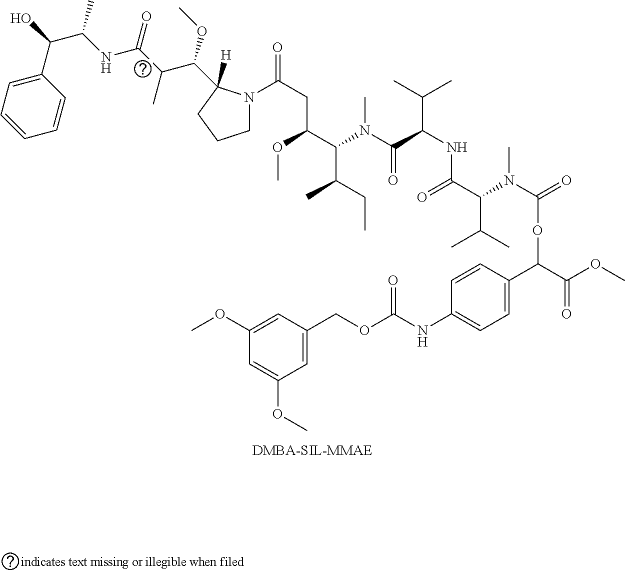 [SMILES: CC[C@@H](C)[C@H]([C@H](CC(=O)N1CCC[C@@H]1[C@@H](OC)C(C)C(=O)N[C@@H](C)[C@H](O)c1ccccc1)OC)N(C)C(=O)[C@H](NC(=O)[C@@H](C(C)C)N(C)C(=O)OC(C(=O)OC)c1ccc(NC(=O)OCc2cc(OC)cc(OC)c2)cc1)C(C)C]

DMBA-SIL-PNP (58.1 mg, 51.9 μmol), MMAE (40.2 mg, 56.0 μmol), and HOBt hydrate (14 mg, 104 μmol) were dissolved in dry DMF (1 mL) and DIPEA (20 μL, 115 μmol) was added. The mixture was shaken at 25° C. for 16 hours. The reaction mixture was purified by loading the reaction mixture directly onto a reverse-phase column and running a gradient of 5-100% acetonitrile (0.1% formic acid) in water (0.1% formic acid). The desired product was obtained as a white solid (25.3 mg, 40% yield, ˜50:50 mixture of diastereomers). (Mixture of diastereomers and rotamers; major peaks reported). ¹H NMR (400 MHz, CDCl3): δ 8.27 (d, J=9.2 Hz, 2H), 8.15 (brs, 3H), 8.01 (s, 1H), 7.48-7.39 (m, 6H), 7.35-7.30 (m, 4H), 6.52 (t, J=2.2 Hz, 3H), 6.42 (q, J=2.5 Hz, 1H), 5.91 (s, 1H), 5.13 (t, J=4.7 Hz, 3H), 4.27-4.14 (m, 1H), 3.87 (s, 1H), 3.79 (s, 6H), 3.77 (s, 3H), 3.74 (s, 1H), 3.54-3.46 (m, 1H), 3.38 (s, 2H), 3.32 (s, 1H), 3.29 (s, 2H), 3.03 (s, 1H), 2.96 (s, 3H), 2.88 (s, 3H), 2.66 (s, 2H), 2.48-2.3 (m, 3H), 2.10-1.98 (m, 3H), 1.88-1.80 (m, 1H), 1.48-1.40 (m, 1H), 1.23 (d, J=7.0 Hz, 3H), 1.05-0.78 (m, 21H). ¹³C NMR (101 MHz, CDCl3): δ 174.9, 168.6, 164.7, 163.1, 161.1, 155.4, 151.9, 145.7, 141.0, 139.5, 138.1, 128.8, 128.2, 127.5, 126.4, 126.2, 125.5, 121.9, 119.0, 115.9, 106.1, 100.3, 82.1, 77.8, 75.7, 72.6, 67.7, 67.2, 61.1, 60.2, 58.0, 55.5, 53.9, 53.2, 51.5, 48.1, 45.1, 37.5, 36.8, 32.8, 31.8, 30.2, 25.9, 25.1, 25.0, 19.0, 17.8, 15.9, 14.5, 14.1, 12.1, 10.7. MS: m/z: calculated for C59H87N6015 [M+H]⁺ 1119.62 found 1119.98.

### Preparation of DMBA-SIL-Mal-MMAE (14)

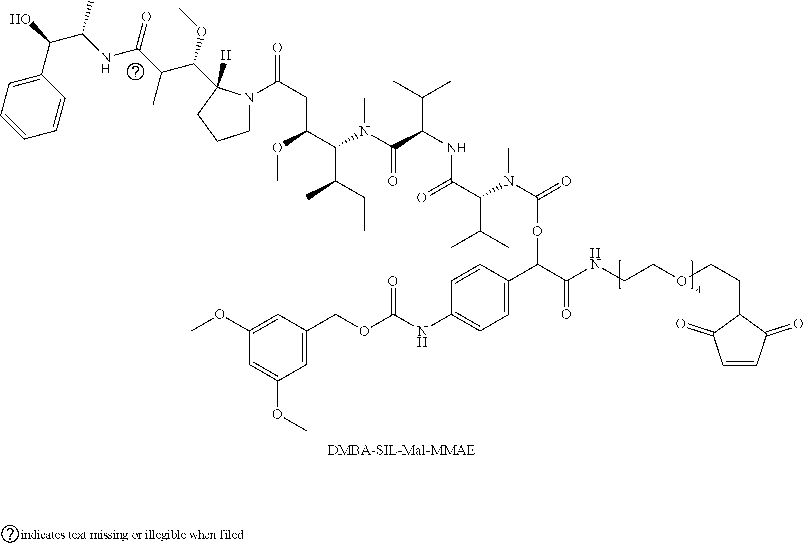

DMBA-SIL-MMAE (12.7 mg, 11.3 μmol) was dissolved in MeOH (2 mL) and an aqueous solution of 0.5 M LiOH (1 mL, 0.5 mmol) was added. This mixture was stirred for 20 minutes, then analyzed by LC-MS to verify the hydrolysis. Amberylst resin (˜100 mg, acidic) was added and the mixture was stirred for 1 minute. The resin was removed by filtration and washed with methanol, which was then removed by rotary evaporation. The resulting residue was dissolved in dry THF (1 mL) and a solution of NH₂-PEG4-Mal TFA in DMF (50 mg/mL, 72 μL, 11.4 μmol) and DIPEA (10 μL, 57.4 μmol) were added. This mixture was then stirred at room temperature for 48 hours. The reaction mixture was purified by reverse-phase chromatography using a gradient of 5-100% acetonitrile (0.1% formic acid) in water (0.1% formic acid). The product was obtained as a colorless, amorphous solid, which was further cleaned by normal phase chromatography using a gradient of 0-15% methanol in DCM. The desired product eluted at a concentration of 12% methanol and the fractions were evaporated by rotary evaporation to provide the product as a white solid (10.6 mg, 67% yield, ˜40:60 mixture of diastereomers). (Mixture of diastereomers and rotamers; major peaks reported). ¹H NMR (400 MHz, CDCl3): δ 7.40-7.32 (m, 10H), 6.66-6.60 (m, 2H), 6.53 (s, 2H), 6.42 (s, 1H), 5.11 (s, 2H), 4.95 (s, 1H), 4.25 (s, 1H), 4.15 (s, 1H), 3.93 (s, 2H), 3.79 (s, 6H), 3.77 (s, 3H), 3.69 (d, J=5.6 Hz, 2H), 3.65-3.45 (m, 24H), 3.42-3.37 (m, 6H), 3.33-3.28 (m, 4H), 3.09 (s, 1H), 3.01-2.92 (m, 5H), 2.92-2.85 (m, 2H), 2.48-2.35 (m, 2H), 2.27-2.15 (m, 1H), 2.10-2.00 (m, 3H), 1.89-1.78 (m, 2H), 1.24 (s, 3H), 1.04-0.75 (m, 21H). HRMS: m/z: calculated for C72H107N8020 [M+H]⁺ 1403.7580 found 1403.7596 (Δ=1.1 ppm).

### Preparation of DMBA-SIL-Dox (8)

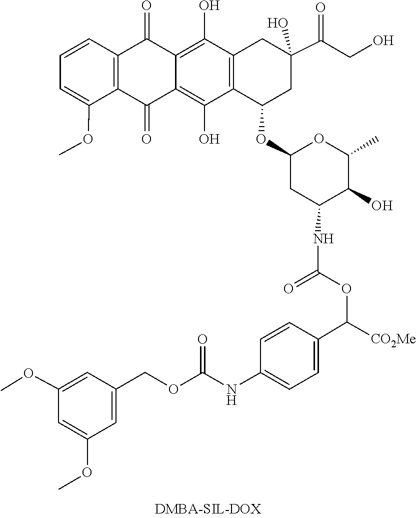 [SMILES: COC(=O)C(OC(=O)N[C@@H]1C[C@@H](O[C@H]2C[C@](O)(C(=O)CO)Cc3c(O)c4c(c(O)c32)C(=O)c2c(OC)cccc2C4=O)O[C@H](C)[C@H]1O)c1ccc(NC(=O)OCc2cc(OC)cc(OC)c2)cc1]

Doxorubicin hydrochloride (100 mg, 172.4 μmol) was added to a solution of DMBA-SIL-PNP (153 mg, 283.1 μmol) and DIPEA (150 μL, 861.1 μmol) in dry DMF (3 mL). The mixture was stirred at room temperature for 18 hours, then diluted with ethyl acetate (200 ml) and washed with 1M HCl, water and brine (200 mL each). The organic phase was then dried with magnesium sulfate and evaporated. The resulting residue was dissolved in DMSO, loaded onto a reverse-phase column, and purified using a gradient of 5-100% acetonitrile (0.1% formic acid) in water (0.1% formic acid). The product was isolated as a red solid (111.6 mg, 69% yield, ˜60:40 mixture of diastereomers). (Mixture of diastereomers and rotamers; major peaks reported). ¹H NMR (400 MHz, CDCl3): δ 13.89 (d, J=11.9, 1H), 13.11 (d, J=3.4 Hz, 1H), 7.92 (s, 1H), 7.72 (s, 1H), 7.41-7.24 (m, 4H), 7.22 (d, J=9.2 Hz, 1H), 6.54-6.46 (m, 3H), 6.39 (d, J=19.19 Hz, 1H), 5.76 (s, 1H), 5.48 (s, 1H), 5.18 (s, 1H), 5.12 (s, 1H), 5.06 (d, J=5.0 Hz, 2H), 4.76 (s, 2H), 4.57-4.52 (m, 1H), 4.15-4.08 (m, 1H), 4.01 (d, J=5.9 Hz, 2H), 3.85 (s, 1H), 3.80-3.72 (m, 10H), 3.66 (s, 1H), 3.61 (s, 1H), 3.16 (d, J=18.7 Hz, 2H), 2.86 (d, J=18.1 Hz, 1H), 2.32 (d, J=13.8 Hz, 1H), 2.12 (d, J=10.2 Hz, 1H), 1.85 (d, J=0.9 Hz, 2H), 1.29 (d, J=12.3 Hz, 3H). ¹³C NMR (101 MHz, CDCl3): δ 214.0, 186.9, 170.1, 169.2, 161.0, 156.2, 155.5, 154.7, 153.3, 138.9, 138.2, 135.8, 135.3, 133.6, 128.6, 128.5, 128.1, 120.7, 119.8, 118.9, 118.5, 111.5, 106.6, 106.0, 100.9, 100.2, 74.4, 69.7, 69.4, 67.4, 67.0, 65.6, 56.6, 55.42, 52.9, 52.8, 47.4, 46.4, 35.6, 33.9, 29.9, 16.9. MS: m/z: calculated for C47H47N2019 [M−H]− 943.28 found 943.64.

### Preparation of DMBA-SIL-Mal-DOX (9)

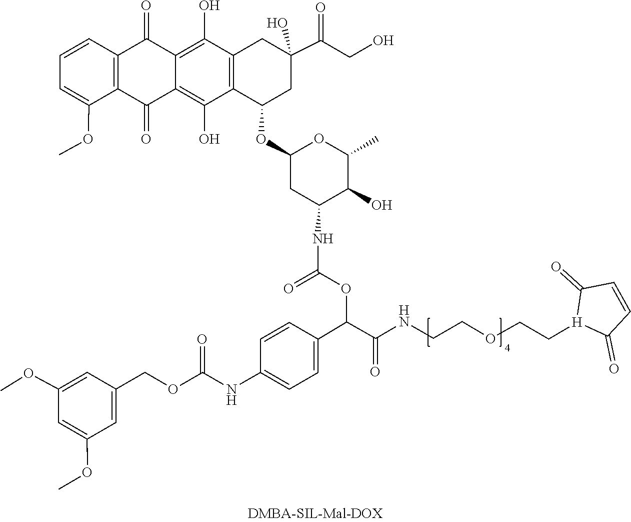

DMBA-SIL-Dox (111.6 mg, 118.2 μmol) was dissolved in MeOH (10 mL) and an aqueous solution of 0.5 M LiOH (5 mL, 0.5 mmol) was added. This mixture was stirred for 25 minutes. Amberylst resin (˜200 mg, acidic) was added and the mixture was stirred for 1 minute. The resin was removed by filtration and washed with methanol, which was then removed by rotary evaporation. The resulting residue was dissolved in dry THF (6 mL) and a solution of NH2-PEG4-Mal TFA in DMF (100 mg/mL, 350 μL, 110.6 μmol), HBTU (63.4 mg, 167.1 μmol) and DIPEA (100 μL, 574 μmol) were added. This mixture was then stirred at room temperature for 18 hours before purification by reverse phase chromatography using a gradient of 5-100% acetonitrile in water (0.1% formic acid). The product was isolated as a red solid (48.5 mg, 33% yield, ˜60:40 mixture of diastereomers). (Mixture of diastereomers and rotamers; major peaks reported). ¹H NMR (400 MHz, CDCl3): δ 13.87 (d, J=10.6 Hz, 1H), 13.12 (d, J=3.8 Hz, 1H), 8.02 (s, 2H), 7.74 (s, 2H), 7.39-7.29 (m, 6H), 7.21 (s, 2H), 6.66 (d, J=8.9 Hz, 1H), 6.62 (s, 1H), 6.52-6.36 (m, 6H), 5.45 (s, 1H), 5.19 (s, 1H), 5.10-5.02 (m, 4H), 4.74 (s, 2H), 4.10-3.96 (m, 6H), 3.84-3.82 (m, 1H), 3.78-3.52 (m, 16H), 3.24-3.09 (m, 2H), 2.94-2.87 (m, 2H), 2.32-2.28 (m, 1H), 2.09 (d, J=11.4 Hz, 1H), 1.99 (s, 1H), 1.91-1.73 (m, 2H), 1.25 (d, J=4.5 Hz, 6H). ¹³C NMR (101 MHz, CDCl3): δ 214.0, 186.9, 186.6, 170.9, 162.7, 161.0, 156.3, 155.6, 153.3, 138.3, 138.3, 135.8, 135.4, 134.2, 133.6, 128.6, 128.5, 128.4, 120.8, 119.8, 118.9, 118.5, 111.5, 111.4, 106.6, 106.0, 100.9, 100.5, 100.3, 100.2, 70.6, 70.5, 70.1, 70.0, 69.8, 69.6, 69.2, 67.9, 67.4, 67.0, 65.6, 56.7, 55.5, 52.7, 47.4, 46.5, 39.3, 37.2, 35.7, 33.9, 29.8, 17.1, 17.0. HRMS: m/z: calculated for C60H72N5024 [M+NH4]+ 1246.4562 found 1246.4565 (Δ=0.2 ppm).

### Preparation of 4-azido-2,3,5,6-tetrafluorobenzyl (4-nitrophenyl) carbonate (pATFB-PNP)

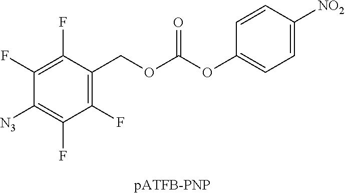 [SMILES: [N-]=[N+]=Nc1c(F)c(F)c(COC(=O)Oc2ccc([N+](=O)[O-])cc2)c(F)c1F]

4-azido-2,3,5,6-fluorobenzyl alcohol (100.2 mg, 0.452 mmol) and bis-4-nitrophenyl carbonate (138 mg, 0.452 mmol) were dissolved in DCM (10 mL). DIPEA (80 μL, 0.454 mmol) was added and the mixture was shaken at room temperature for 18 hours. The crude mixture was evaporated and redissolved in toluene, then purified using a gradient of 0-25% ethyl acetate in hexanes, followed by a flush using 10% MeOH in DCM. The desired compound was isolated as a pale-yellow solid (101.7 mg, 58% yield). ¹H NMR (400 MHz, CDCl3): δ 8.28 (d, J=7.1 Hz, 2H), 7.40 (d, J=9.2 Hz, 2H), 5.39 (s, 2H). ¹³C NMR (101 MHz, CDCl3): δ 155.3, 152.1, 147.1, 145.7, 144.6, 139.3, 125.5, 121.8, 108.2, 57.8. ¹⁹F NMR (376 MHz, CDCl3): δ −142.2, −151.4.

### Preparation of pATFB-MMAE

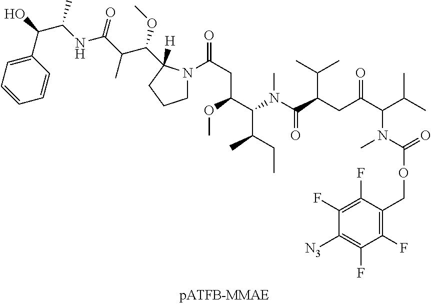 [SMILES: CC[C@@H](C)[C@H]([C@H](CC(=O)N1CCC[C@@H]1[C@@H](OC)C(C)C(=O)N[C@@H](C)[C@H](O)c1ccccc1)OC)N(C)C(=O)[C@H](CC(=O)C(C(C)C)N(C)C(=O)OCc1c(F)c(F)c(N=[N+]=[N-])c(F)c1F)C(C)C]

MMAE (50 mg, 69.6 μmol) and pATFB-PNP (40.3 mg, 104 μmol) were dissolved in dry DMSO (2.5 mL). DIPEA (18 μL, 104 μmol) was added and the mixture was shaken at 30° C. for 19 hours. A further 1.5 eq (18 μL) of DIPEA was added and the reaction was left stirring for a further 20 hours. The reaction mixture was then treated with ethanolamine (20 μL, 331 μmol) to quench unreacted pATFB-PNP. This mixture was stirred for 1 hour, then found to be free of PNP carbonate (by LC-MS) and loaded directly onto a reverse-phase column and purified using a gradient of 5-100% acetonitrile in water (0.1% formic acid). The product was purified as a white solid (32.5 mg, 48% yield). ¹H NMR (400 MHz, CDCl3): δ 7.66 (brs, 2H), 7.32 (s, 3H), 6.65 (s, 1H), 5.29 (q, J=12.1 Hz, 1H), 5.17 (t, J=9.4 Hz, 1H), 5.08-4.85 (m, 1H) 4.80-4.60 (m, 1H), 4.27 (s, 1H), 4.17-4.05 (m, 2H), 3.79 (s, 1H), 3.61-3.31 (m, 5H), 3.19-3.01 (m, 2H), 2.91 (s, 1H), 2.82 (s, 1H), 2.71 (d, J=7.3 Hz, 2H), 2.63-2.53 (m, 1H), 2.21-1.90 (m, 4H), 1.24 (s, 4H), 1.10-0.91 (m, 7H), 0.90-0.75 (m, 10H). ¹³C NMR (101 MHz, CDCl3): δ 175.6, 171.7, 170.0, 159.4, 159.0, 158.6, 156.6, 55.4, 147.0, 144.5, 143.7, 141.7, 140.9, 139.4, 128.3, 127.6, 126.5, 124.9, 120.8, 119.3, 116.4, 114.8, 113.6, 110.4, 81.7, 78.9, 75.6, 65.3, 61.2, 58.3, 57.7, 57.1, 55.1, 54.4, 52.5, 44.4, 40.1, 36.5, 33.2, 31.1, 30.0, 29.8, 26.8, 26.2, 25.8, 25.0, 19.4, 18.6, 17.7, 16.1, 14.7, 13.8, 10.8. ¹⁹F NMR (376 MHz, CDCl3): δ −142.6, −151.8. MS: m/z calculated for C47H69F4N809+ [M+H]⁺ 964.5045, found 965.5118.

### Preparation of pATFB-DOX

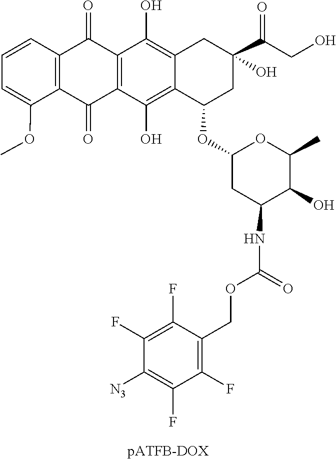 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2c(F)c(F)c(N=[N+]=[N-])c(F)c2F)[C@H](O)[C@H](C)O1]

pATFB-PNP (98.2 mg, 165 μmol) and doxorubicin monohydrate (50 mg, 89.0 μmol) were dissolved in dry DMF (1 mL) and DIPEA (25 UL, 143.5 μmol) added dropwise. After 1 hour shaking at room temperature, LC-MS showed no remaining doxorubicin, so ethanolamine (25 μL, 409.3 μmol) was added and shaking continued for 1 hour to react with any remaining pATFB-PNP. The mixture was then loaded directly onto a reverse-phase column and purified with a gradient of S-100% acetonitrile in water (0.1% formic acid). The product was isolated as a red solid (17.8 mg, 27% yield). ¹H NMR (400 MHz, CDCl₃): δ 13.99 (s, 1H), 13.26 (s, 1H), 8.26 (t, J=9.4 Hz, 1H), 8.04 (s, 2H), 7.81 (t, J=8.4 Hz, 1H), 7.42 (d, J=8.4 Hz, 1H), 5.22 (s, 1H), 5.35-5.17 (m, 4H), 5.13 (s, 2H), 4.77 (s, 2H), 4.16 (d, J=, 6.9 Hz, 1H), 4.10 (s, 3H), 3.88 (brs, 1H), 3.74 (s, 1H), 3.68 (s, 2H), 3.39 (t, J=6.48 Hz, 1H), 3.29 (d,)=18.0 Hz, 1H), 2.99 (s, 2H), 2.91 (s, 2H), 2.34 (d, J=14.6 Hz, 2H), 2.18 (dd, J=14.66, 4.3 Hz, 2H), 2.03 (s, 1H), 1.88 (dd, J=13.1, 3.9 Hz, 1H), 1.80 (td, J=13.4, 4.3 Hz, 1H), 1.54 (d, J=6.6 Hz, 1H), 1.46 (d, J=6.6 Hz, 1H), 1.30 (d, J=7.6 Hz, 4H). ¹³C NMR (101 MHz, CDCl3): δ 213.9, 187.3, 186.8, 171.9, 156.3, 155.8, 154.9, 144.4, 136.0, 125.6, 120.9, 120.0, 118.6, 111.7, 100.8, 69.9, 69.6, 67.3, 65.7, 56.8, 54.0, 47.3, 35.8, 34.1, 30.3, 17.5, 15.9. ¹⁹F NMR (376 MHz, CDCl3): δ −142.33, −151.76. MS: m/z calculated for C35H29F4N4013 [M−H]− 789.17, found 789.72.

### Preparation of methyl tetrafluorobenzyl)oxy) carbonyl)amino)phenyl)-2-(((4-nitrophenoxy) carbonyl)oxy)acetate (pATFB-SIL-PNP)

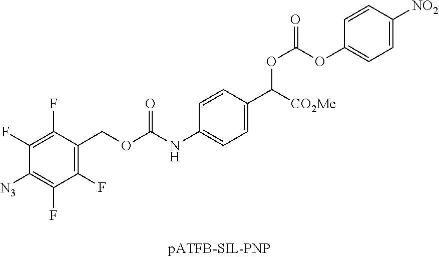 [SMILES: COC(=O)C(OC(=O)Oc1ccc([N+](=O)[O-])cc1)c1ccc(NC(=O)OCc2c(F)c(F)c(N=[N+]=[N-])c(F)c2F)cc1]

### Step 1: Preparation of methyl 2-(4-((((4-azido-2,3,5,6-tetrafluorobenzyl)oxy) carbonyl)amino)phenyl)-2-hydroxyacetate

4-azido-2,3,5,6-tetrafluorobenzyl alcohol (60 mg, 155 μmol) and SIL (28.3 mg, 156.4 μmol) were dissolved in DMF (6 mL) and DIPEA (27 μL, 155 μmol) was added. The reaction mixture was stirred at 30° C. for 48 hours, evaporated by rotary evaporation and redissolved in toluene (5 mL). The product was purified by loading the mixture directly onto at normal phase column and running a gradient of 0-20% EtOAc in Hexanes. The product was isolated as a white solid (20 mg, 30% yield). ¹H NMR (400 MHz, CDCl3): δ 7.36 (s, 4H), 6.70 (s, 1H), 5.28 (s, 2H), 5.14 (s, 1H), 3.75 (s, 1H). ¹³C NMR (101 MHz, CDCl₃): δ 174.1, 152.4, 137.5, 133.7, 127.5, 118.9, 109.8, 72.4, 54.2, 53.1. MS: m/z calculated for C₁₇H₁₁F₄N₄O₉ [M−H]⁻ 427.07 found 427.37.

### Step 1: Preparation of pATFB-SIL-PNP

pATFB-SIL (17.8 mg, 41.6 μmol) and bis(4-nitrophenyl) carbonate (13.0 mg, 42.7 μmol) were dissolved in DCM (1 mL). DIPEA (10 μL, 57.4 μmol) was added and the reaction was stirred at 45° C. for 20 hours. The reaction mixture was then diluted with DCM (50 mL) and washed sequentially with 1M HCl, water, and brine (50 mL each). The organic phase was dried with magnesium sulfate, evaporated, and purified by normal-phase chromatography using a gradient of 0-50% EtOAc in hexanes. The product was obtained as a white solid (13.3 mg, 54% yield). ¹H NMR (400 MHz, CDCl3): δ 8.27 (d, J=7.0 Hz, 2H), 7.48-7.40 (m, 6H), 7.17 (s, 1H), 5.90 (s, 1H), 5.28 (s, 2H), 3.76 (s, 3H). ¹³C NMR (101 MHz, CDCl3): δ 168.5, 155.4, 151.9, 145.7, 139.2, 128.8, 127.6, 125.5, 121.9, 119.1, 77.8, 53.2, 41.0. ¹⁹F NMR (376 MHz, CDCl3): δ −142.1, −151.5.

### Preparation of pATFB-SIL-MMAE

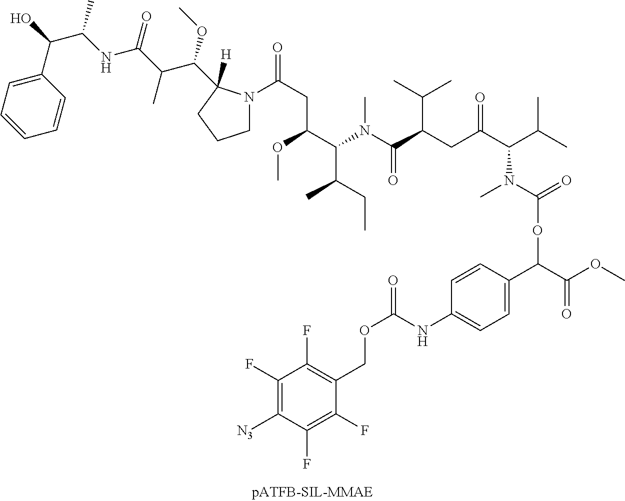 [SMILES: CC[C@@H](C)[C@H]([C@H](CC(=O)N1CCC[C@@H]1[C@@H](OC)C(C)C(=O)N[C@@H](C)[C@H](O)c1ccccc1)OC)N(C)C(=O)[C@H](CC(=O)[C@H](C(C)C)N(C)C(=O)OC(C(=O)OC)c1ccc(NC(=O)OCc2c(F)c(F)c(N=[N+]=[N-])c(F)c2F)cc1)C(C)C]

MMAE (20.6 mg, 28.7 μmol) was dissolved in dry DMF (1 mL) and pATFB-SIL-PNP (15.4 mg, 26.0 μmol) was added. Once everything had dissolved, DIPEA (10 μL, 57.4 μmol) was added and the reaction was stirred at 30° C. for 16 hours. The reaction mixture was then loaded directly onto a reverse phase column and purified using a gradient of 5-100% acetonitrile (0.1% formic acid) in water (0.1% formic acid) to provide the product as a white solid (13.7 mg, 45% yield). (Mixture of diastereomers and rotamers; major peaks reported). ¹H NMR (400 MHz, CDCl3): δ 8.18 (d, J=8.9 Hz, 2H), 8.12 (d, J=8.8 Hz, 1H), 7.62-7.33 (m, 5H), 6.97 (s, 3H), 5.28 (s, 1H), 5.02 (s, 1H), 4.70-4.63 (m, 1H), 4.51 (s, 1H), 4.26 (s, 1H), 3.76 (s, 6H), 3.40 (s, 1H), 3.31 (s, 2H), 3.02 (s, 1H), 2.70 (s, 1H), 2.44 (s, 1H), 2.10-1.75 (m, 3H), 1.24 (s, 1H), 1.05-0.78 (m, 9H). ¹³C NMR (101 MHz, CDCl3): δ 169.4, 168.9, 162.7, 162.0, 161.7, 149.2, 146.7, 144.2, 143.5, 142.6, 142.4, 141.7, 141.2, 139.2, 129.7, 129.4, 129.3, 128.9, 128.5 128.4, 128.1, 128.0, 127.3, 126.3, 126.3, 126.1, 123.8, 120.1, 116.5, 115.9, 115.5, 115.4, 112.0, 78.4, 78.0, 72.2, 58.4, 58.0, 53.5, 53.4, 53.1, 43.7, 35.5. ¹⁹F NMR (376 MHz, CDCl3): δ −142.2, −151.6. MS: m/z calculated for C57H77F4N9013 [M+H]+ 1171.56, found 1172.97.

### Preparation of pATFB-SIL-Mal-MMAE

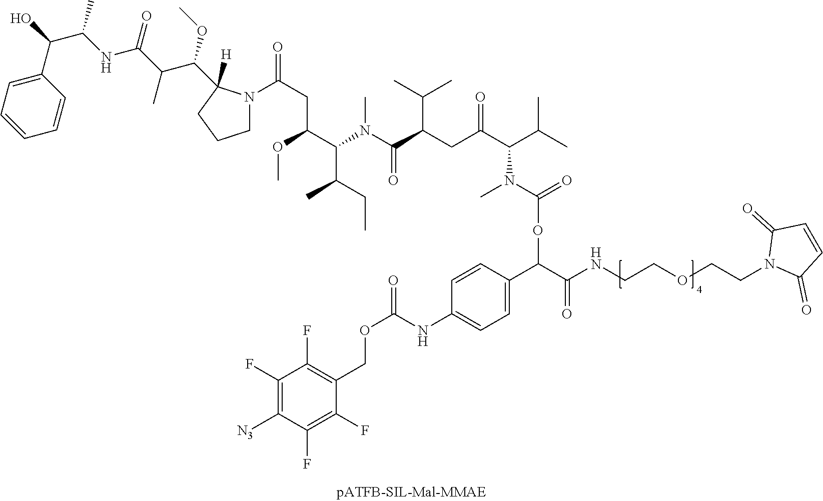

pATFB-SIL-MMAE (28 mg, 23.9 μmol) was dissolved in MeOH (5 mL) and 0.5M LiOH (0.2 mL) was added. The mixture was stirred for 25 minutes, then analyzed by LC-MS. After it was determined that most of the material had formed the hydrolysis intermediate, the mixture was quenched by addition of acidic Amberlyst resin (˜1 g). The suspension containing the resin was filtered and washed with methanol, and the solvent was removed via rotary evaporation. The resulting residue was dissolved in dry DMF (2 mL) and Mal-PEG4-amine TFA salt (10 mg, 31.6 μmol), HBTU (20.2 mg, 53.2 μmol) and DIPEA (15 μL, 86.1 μmol) were added. This mixture was then stirred at room temperature for 16 hours, then added directly to a reverse phase column and purified using a gradient of 5-100% acetonitrile in water (0.1% formic acid). The desired product was obtained as a white solid (12.2 mg, 35% yield). (Mixture of diastereomers and rotamers; major peaks reported). ¹H NMR (400 MHz, CDCl3): δ 7.52 (s, 1H), 7.37-7.30 (m, 8H), 7.04 (s, 2H), 6.69 (s, 1H), 5.32-5.15 (m, 3H), 4.95 (s, 1H), 4.26 (s, 1H), 4.16-4.08 (m, 4H), 3.71 (s, 2H), 3.68-3.50 (m, 14H), 3.41 (s, 4H), 3.31 (s, 3H), 3.10-2.85 (m, 5H), 2.80 (d, J=7.6 Hz, 1H), 2.5-2.3 (m, 2H), 2.24 (s, 1H), 2.02 (s, 2H), 1.85 (s, 2H), 1.11 (s, 1H), 1.03-0.78 (m, 24H). ¹⁹F NMR (376 MHz, CDCl3): δ −144.6, −154.0. MS: m/z calculated for C70H98F4N11O18 [M+H]+ 1456.7022, found 1457.16.

### Preparation of pATFB-SIL-DOX

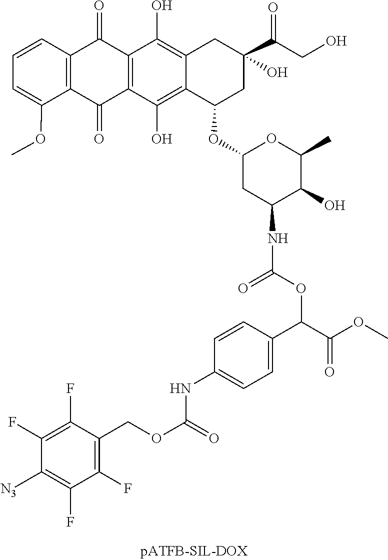

Doxorubicin (45 mg, 82.6 μmol) and pATFB-SIL-PNP (35 mg, 59.0 μmol) were dissolved in dry DMF (4 mL) and DIPEA (20 μL, 115.0 μmol) was added. The mixture was then shaken for 18 hours on a rotary shaker at room temperature. Ethanolamine (20 μL, 327 μmol) was added to react with any remaining pATFB-SIL-PNP and the mixtures were incubated for an hour at room temperature before they were diluted with DCM (50 mL each) and washed with 1M HCl (50 mL each). The organic phase was then dried over magnesium sulfate and evaporated. Both crude residues were then dissolved in DMSO (1 mL), loaded onto a reverse phase column, and purified using a gradient of 5-100% acetonitrile (0.1% formic acid). Fractions containing the desired product were evaporated to provide the product as a red solid (21.4 mg, 26% yield). (Mixture of diastereomers and rotamers; major peaks reported). ¹H NMR (400 MHz, CDCl3): δ 13.90 (d, J=8.1 Hz, 1H), 13.16 (dd, J=9.3, 3.8 Hz, 1H), 8.03 (s, 1H), 7.97 (dd, J=7.7, 4.7 Hz, 1H), 7.75 (t, J=7.8 Hz, 1H), 7.39-7.30 (m, 4H), 7.24-20 (m, 1H), 5.74 (t, J=8.7 Hz, 1H), 5.65-5.55 (m, 1H), 5.47 (s, 1H), 5.23 (t, J=7.2 Hz, 3H), 4.76 (s, 1H), 4.74 (s, 1H), 4.32 (d, J=13.8 Hz, 1H), 4.03 (s, 4H), 3.86-3.78 (m, 1H), 3.69-3.61 (m, 2H), 3.21 (dd, J=18.7, 5.8 Hz, 1H), 2.95 (d, J=18.3 Hz, 1H), 2.45-2.29 (m, 1H), 2.14 (t, J=14.7 Hz, 1H), 2.00 (s, 1H), 1.90-1.80 (m, 2H), 1.28 (dd, J=13.0, 6.5 Hz, 3H), 1.20-1.09 (m, 1H). ¹³C NMR (101 MHz, CDCl3): δ 213.7, 186.6, 162.9, 161.0, 156.2, 155.6, 135.8, 133.5, 128.5, 127.4, 127.2, 119.8, 118.5, 111.5, 100.6, 65.5, 56.6, 54.11, 47.3, 35.7, 33.8, 29.9, 16.8. ¹⁹F NMR (376 MHz, CDCl3): δ −142.2, −151.6. MS: m/z calculated for C45H39F4N5017 [M−H]− 996.2204, found.

### Preparation of pATFB-SIL-Gard

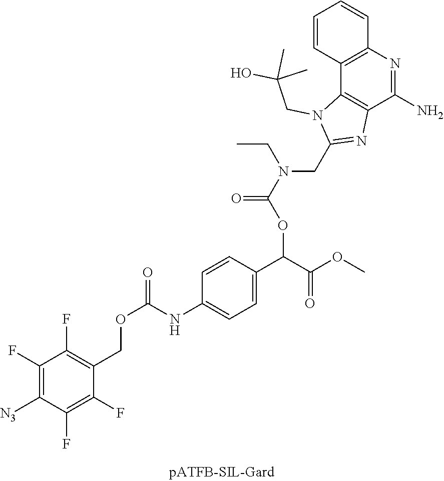 [SMILES: CCN(Cc1nc2c(N)nc3ccccc3c2n1CC(C)(C)O)C(=O)OC(C(=O)OC)c1ccc(NC(=O)OCc2c(F)c(F)c(N=[N+]=[N-])c(F)c2F)cc1]

pATFB-SIL-PNP (10 mg, 16.8 μmol) and gardiquimod diTFA (15 mg, 27.7 μmol) were dissolved in dry DMF (0.5 mL) and DIPEA (10 μL, 57.4 μmol) were added. The mixture was shaken at room temperature for 16 hours, diluted in EtOAc (10 mL), and washed with 0.1M NaOH and brine (10 mL each). The organic phase was then evaporated and the resulting residue was dissolved in DMSO, loaded onto a reverse phase column and purified using a gradient of 5-100% acetonitrile in water (0.1% formic acid). The desired product was obtained as a white solid (6.2 mg, 48% yield). ¹H NMR (400 MHz, MeOD-d4): δ 8.39 (s, 2H), 7.78 (d, J=7.8 Hz, 1H), 7.71 (d, J=7.3 Hz, 1H), 7.51 (s, 1H), 7.41-7.32 (m, 1H), 5.32 (s, 1H), 4.62 (s, 1H), 3.75-3.50 (m, 12H), 3.15 (s, 1H), 3.02 (s, 1H), 2.88 (s, 1H), 2.68 (s, 2H), 1.40-1.31 (m, 10H). ¹⁹F NMR (376 MHz, MeOD-d4): δ −74.1, −75.9. MS: m/z calculated for C35H32F4N9O7 [M−H]− 766.2366, found 766.47.

### Preparation of pATFB-SIL-Mal-Gard

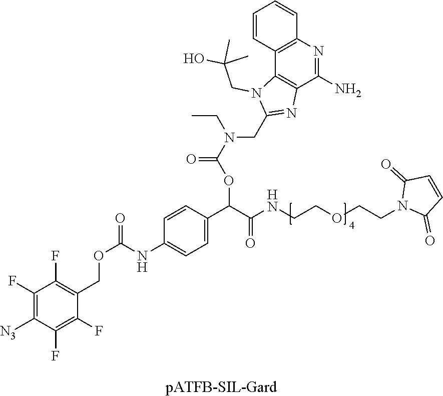

pATFB-SIL-Gard (6.2 mg, 8 μmol) was dissolved in MeOH (1 mL) and 0.5M LiOH (0.2 mL) was added. The mixture was stirred for 25 minutes, then analyzed by LC-MS. After it was determined that most of the material had formed the hydrolysis product, the mixture was quenched by addition of acidic Amberlyst resin. The mixture was then stirred for 1 minute before the resin was removed by filtration and the solvent removed by evaporation. The resulting residue was dissolved in dry DMF (2 mL) and Mal-PEG4-amine (4 mg, 12.6 μmol), HBTU (5 mg, 13.2 μmol) and DIPEA (5 μL, 28.7 μmol) were added. This mixture was then stirred at room temperature for 16 hours. The reaction mixture was added directly to a reverse phase column and purified using a gradient of 5-100% acetonitrile in water (0.1% formic acid). The desired product was obtained as a white solid (1.5 mg, 18% yield). ¹H NMR (400 MHz, MeOD-d4): δ 8.48 (s, 2H), 7.72 (s, 2H), 7.61 (s, 1H), 7.47 (s, 2H), 7.32 (t, J=, 1H), 7.08 (s, 2H), 6.78 (s, 1H), 5.30 (s, 1H), 5.23 (s, 1H), 4.63 (s, 1H), 3.76-3.51 (m, 22H), 3.42-3.38 (m, 1H), 3.16 (s, 1H), 3.02 (s, 1H), 2.68 (s, 2H). ¹⁹F NMR (376 MHz, MeOD-d4): δ −74.0, −75.9. MS: m/z calculated for C48H54F4N11012 [M+H]+ 1052.3884, found 1052.66.

### Preparation of Conjugates

BSA: To a solution of bovine serum albumin (BSA, 47.1 mg in 1 mL PBS) was added 100 μL of a 10 mg/mL solution of Mal-containing prodrug (9 or 11) in DMF. These mixtures were then incubated at room temperature with gentle mixing for 3 hours, followed by removal of the unreacted prodrugs by spin filtration (20,000×g for 5 minutes, ×3) using 30,000 kDa MWCO spin filters (Amicon, 0.5 mL). The conjugates were further purified by size-exclusion chromatography in PBS using a SEC column on an Agilent 1260 HPLC. Collected fractions eluting between 8.5-10.5 minutes were concentrated by further spin filtration to provide a final conjugate concentration of 10 mg/mL in PBS. Alb is generally well tolerated in patients, given at high doses, but can undergo accelerated clearance if extensively modified; therefore, we performed conjugation with a low degree of labeling, achieving on average 0.12 drug molecules per Alb molecule (Table S1).

Antibody: Conjugates of the DMBA-SIL-DOX/MMAE prodrugs to an anti-epidermal growth factor receptor (EGFR) antibody (BioXcell, mab225) were prepared by first reducing the disulfide bonds in the antibody by incubating in 100 μM tris(2-carboxyethyl) phosphine hydrogen chloride (TCEP-HCl) for one hour at room temperature, followed by removal of the reducing agent by spin filtration (10,000×g, 50,000 kDa MWCO, Amicon/Sigma). While leaving a small fraction of the TCEP-HCl (˜50 μL) to maintain the reduced state of the antibody, the prodrugs were added to separate aliquots and incubated at room temperature for 2 hours, after which unreacted prodrug was removed by further spin filtration (10,000×g, x 3, 50,000 kDa MWCO). The degree of labeling was estimated to be on average 5.6 drug molecules per antibody calculating following manufacturer guidelines and using a NanoDrop spectrophotometer (Table S1).

TABLE S1
Values used to estimate the drug loading of BSA-DMBA-SIL-
DOX and IgG-DMBA- SIL-DOX. The absorbance values shown here
were measured using a NanoDrop 1000 (Thermo Scientific).

| | BSA | IgG |
|---|---|---|
| A₂₈₀ | 0.63 | 0.83 |
| A₄₈₀ | 0.39 | 0.11 |
| ε BSA | 43824 | 210000 |
| ε′ Dox | 13500 | 13500 |
| Correction Factor | 0.753 | 0.753 |
| [BSA] (M) | 3.94E−05 | 1.46E−06 |
| DOL | 0.118 | 5.57 |

### Drug Release Studies

Determination of drug release efficiency was performed by preparing solutions of each prodrug in deionized water (0.1% DMF) to a final concentration of 10 μM. Vacuum degassing was performed by gently stirring the samples at ˜200 mbar for thirty minutes at room temperature, while degassing by argon was performed by bubbling ultra-high purity grade gas (Airgas, Lynn, MA, 01902, USA) into the solution at a rate of approximately 5 mL s-1. After these preparations, X-ray irradiation was performed using a Precision (Madison, CT, USA) X-Rad320 at a rate of 385±10 cGy/min until the desired dosage was achieved. Gamma irradiation was similarly performed on a dual source 137Cs Gammacell 40 Exactor (Best Theratronics) with a dose rate of roughly 50 cGy/min. After irradiation or other treatment, the concentration of the released drug and the intact prodrug were determined by LC-MS on a Waters instrument equipped with a Waters 2424 ELS Detector, Waters 2998 UV-Vis Diode array Detector, and a Waters 3100 Mass Detector. Drug concentrations were determined by comparing the AUC from the ELSD or isolated mass chromatographs (+ESI, 718.8 Da for MMAE, 544.4 Da for DOX) of the analyzed sample to a standard calibration curve.

For the measurement of released products relative to the X-ray dosage, DMBA-SIL-MMAE protein conjugate was prepared to a concentration of 50 μM in PBS (0.1% DMF, 5 mL), then purged with ultra-pure grade argon (5 mL s-1) for 15 minutes. The solution was then X-ray irradiated using the X-Rad320 to deliver 8 Gy irradiation and analyzed by LC-MS as described above.

Determination of the oxygen concentrations for each degassing condition was achieved through the use of the Winkler titration method 46 and is reported as parts per million (ppm, mg O₂ per kg dI H₂O). Briefly, water samples were degassed either by vacuum or argon purging as described above, with untreated deionized water was used as a control. To the samples were then added solutions of manganese sulfate monohydrate (100 μL, 2 mM) and alkaline potassium iodide (100 μL, 12.5 M NaOH, 0.8 M KI, 0.15 M NaN₃). The solutions were mixed and let stand for 15 minutes before concentrated sulfuric acid (100 μL) was added and again mixed. Addition of 100 μL of a starch solution (50 mg mL⁻¹) turned the solution blue, and the mixture was then titrated with a solution of sodium thiosulfate (2.5 mM) until it reached a colorless endpoint. The volume of the thiosulfate solution added (in mL) was equivalent to the initial concentration of dissolved oxygen in ppm.

### Cytotoxicity Assays

TBP-3743 (derived from a genetically engineered mouse model of anaplastic thyroid cancer) and 8505c (human anaplastic thyroid cancer) are described previously 59, as are HT1080-EB3-mApple cells 52. Raw264.7 (mouse macrophage model, Raw-MΦ) were from ATCC. MC38 cells provided by M. Smyth (Peter MacCallum Cancer Centre, Victoria, Australia). iKras cells were derived from a genetically engineered mouse model of KrasG12D pancreatic adenocarcinoma and were routinely cultured in Dulbecco's Modified Eagle Medium/Nutrient Mixture F-12 media (DMEM/F12, Invitrogen) supplemented with 2 μg mL-1 doxycycline (Sigma) to maintain mutant Kras expression (provided as a gift from H. Ying, MD Anderson Cancer Center, by way of N. Bardeesy, Massachusetts General Hospital (MGH)) 60. MOC2 (mouse oral cavity squamous cell cancer-Aggressive growth phenotype) (RRID:CVCL_ZD33) cell lines from Kerafast and were cultured according to provider guidelines using IMDM/F12, supplemented with 5 ng/mL EGF (EMD Millipore), 400 ng/mL hydrocortisone (Sigma Aldrich) and 5 mg/mL insulin (Sigma Aldrich). All cells were routinely evaluated for mycoplasma contamination and cultured following provider guidelines using 10% FBS (Bio-Techne Sales), 100 IU mL-1 penicillin, 100 μg mL-1 streptomycin (Invitrogen), with incubation at 37° C. and 5% CO₂. Cytotoxicity experiments were performed by seeding 5,000 cells per well overnight in a 96-well plate (Corning) before the addition of each drug/conjugate. Empty wells with only media or vehicle-treated cells were used as controls. After addition of the corresponding drugs and a 72 hour incubation, the number of live cells was determined by PrestoBlue (ThermoFisher, USA) staining according to the provider's protocols.

### Cell Imaging

Subcellular localization of DOX in the 8505c cell line was measured using a modified BX63 (Olympus) inverted microscopy system equipped with an environmental chamber and robotic stage. DOX images were collected using a 40× air objective (PLAPO 40×/0.95 numerical aperture), with excitation and emission wavelengths of 489 nm and 508 nm, respectively. All images were processed using cellSens Dimension 3.1.1 (Olympus, USA) and ImageJ 1.53k (NIH, USA) software. Nuclear to cytoplasm fluorescence ratios were calculated and plotted using Excel (Microsoft) and Prism (GraphPad). 20 μM concentration of free (or released) DOX was used, and unirradiated prodrug used the same total (caged and uncaged) DOX concentration as used in the irradiated sample. Following treatment for 72 hours, live cells were immediately imaged by confocal microscopy.

EB3 imaging was performed on an FV1000 confocal laser scanning microscope equipped with a 37° C. heated stage, XLUMPLFLN 20× (NA 1.0) water-immersion objective, 559 nm diode laser, and BA575-620 emission filter (all Olympus America). Cells were treated with 1 μM MMAE, Alb-DMBA-SIL-MMAE, or Alb-DMBA-SIL-MMAE after argon purging and X-ray irradiation (8 Gy) 1 hour prior to imaging. MT tracks were obtained by detecting and linking EB3 comets using the U-track software. 61 Cell masks were constructed using ImageJ. For MT tracks to be included in the downstream analyses, they must pass a strict set of filters: (1) located within a cell boundary, (2) be present in a minimum of 3 consecutive frames, (3) must have a path length less than 10 μm, (4) track persistence must be greater than 0.5 (measured on a scale of 0 to 1, with 1 indicating a line). Additionally tracks with outlier speeds (greater than or less than 1.5*IQR) within their respective cells were not included in the analysis.

### Stability Studies

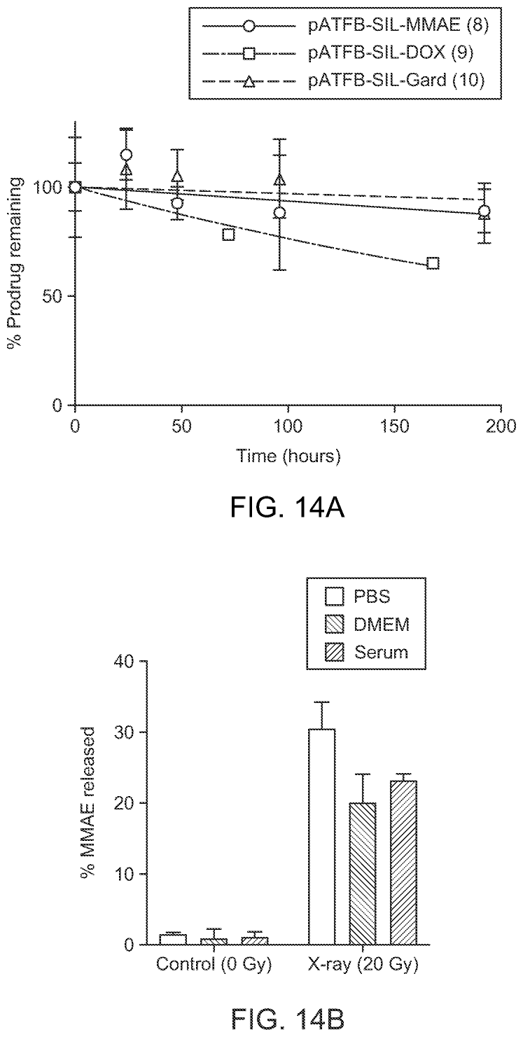

Prodrugs were prepared to concentrations of 10 μM in PBS (pH 7.4, <2% DMF) and incubated at 37° C. to determine the relative stability under physiological conditions (FIG. 14). Aliquots of these solutions were removed after varying time intervals and analyzed via LC-MS on a Waters 3100 Mass Detector with an Xterra MS C18 Column to measure the amount of each prodrug remaining in solution. All measurements were made in triplicate and analyzed in Graphpad Prism to determine the half-lives (991, 257, and 2252 hours were determined for compounds 8, 9, and 10 respectively).

For release experiments in multiple media (FIG. 14B), the prodrug solutions were prepared to a concentration of 10 μM in PBS, DMEM, and FBS Each of these solutions were split into 2 equal aliquots, which were then either X-ray irradiated (20 Gy) or incubated at room temperature 320 KeV X-ray irradiation was performed using a Precision (Madison, CT, USA) X-Rad320 at a rate of 330±10 cGy/min until the desired dosage was achieved. Irradiated samples were incubated at room temperature for 1 hour following irradiation to allow for the drug to fully release. All samples were then analyzed via LC-MS and compared to a previously prepared calibration curve (5 point curve utilizing the [M+H]+ ESI signal) to determine the amount of free drug generated.

### Proton Beam Cleavage

Gamma irradiation was performed on a dual source 137 Cs Gammacell 40 Exactor (Best Theratronics) with a dose rate of roughly 50 cGy/min. Similarly, 225 MeV proton beam irradiation was performed using either conventional or FLASH (120 Gy/s) dose rates on a C230 isochronous cyclotron (Ion Beam Applications SA, Louvain-la-neuve, Belgium) as previously described by Qixian et al. [Qixian et al., 2020, Radiation Research, 194, 656-664] The samples were then analyzed by LC-MS. Results are shown in FIG. 15.

Prodrug activation by various X-ray sources

For radiation release experiments (FIG. 16), 10 μM solutions of each prodrug were prepared in PBS (pH 7.4, <2% DMF) unless otherwise described. Prodrug solutions were split into aliquots of irradiated samples and non-irradiated controls, which would be incubated at room temperature until further analysis. For samples requiring degassing, vacuum degassing was performed by gently stirring the samples at 200 mbar for 30 minutes prior to irradiation, while argon degassing was performed by bubbling ultrahigh-purity grade gas (Airgas, Lynn, MA, USA) into the solution for 15 minutes. X-ray irradiation was performed using a Precision (Madison, CT, USA) X-Rad320 at a rate of 330±10 cGy/min until the desired dosage was achieved and the following LC-MS analysis was performed as described above.

Prodrug Activation with and without SIL and Using X-Ray Versus Radionuclide

For radiation release experiments (results shown in FIG. 17), 10 μM solutions of each prodrug were prepared in PBS (pH 7.4, <2% DMF) unless otherwise described. Prodrug solutions were split into aliquots of irradiated samples and non-irradiated controls, which would be incubated at room temperature until further analysis. X-ray irradiation was performed using a Precision (Madison, CT, USA) X-Rad320 at a rate of 330±10 cGy/min until the desired dosage was achieved. For radioisotope incubation, Cu-64 was obtained from the University of Wisconsin Department of Medical Physics and diluted to a concentration of 30 mCi/mL in sodium citrate buffer (pH 5). From this solution, 300 μCi aliquots were added to the corresponding prodrug solutions, which were then incubated at room temperature while protected from light. After 10 half-lives had passed the samples were analyzed by LC-MS as described above.

### Prodrug Activation in Live Cells and Tumor Tissues

HT1080 cells stably expressing H2B-mApple were prepared as previously described (HT1080 H2B-mApple [Dubach et al., 2017, Nature Chemical Biology, 13, 168-173]). Intracellular release of MMAE from the MSA conjugate was measured by preparing confluent 15 cm culture plates of HT1080-mApple cells and adding the conjugate to a final concentration of 10 μM. These plates were incubated for 24 hours, then X-ray irradiated (10 Gy) and the cells were washed 3 times with PBS. Cell lysis buffer (Invitrogen, 100 μL) was then added to each plate and a cell scraper was used to dislodge the cells. The resulting cell suspension was kept on ice for 30 minutes before a solution of 10% silver nitrate in acetonitrile was added (5 fold dilution). After incubating for a further 10 minutes on ice, the suspensions were centrifuged (10,000 g for 5 min) and the supernatant was analyzed via LC-MS to determine the percentage of MMAE released. A comparison of the various prodrug conjugates was performed by preparing confluent HT1080-mApple cells in a 6-well plate. The cells were then treated with either free drug or the corresponding MSA conjugate, lysed and analyzed as described above (results shown in FIG. 18).

All animal research was performed in accordance with guidelines from the Institutional Subcommittee on Research Animal Care. MC38 tumors were generated by injecting 106 cells into C57BL/6 mice on either flank. These mice were then treated with MSA-pATFB-SIL-MMAE conjugate via tail vein injection (0.2 mg prodrug per mouse). After 24 hours, these mice were X-ray irradiated (10 Gy) on one of their tumors, leaving the contralateral tumor as a negative control. The tumors were then harvested and minced in 100 μL lysis buffer (Invitrogen). The lysate was stored on ice for 1 hour before it was diluted 5-fold with 10% silver nitrate in acetonitrile. This suspension was incubated on ice for 10 minutes, then centrifuged (10,000 g for 5 min) and the supernatant was analyzed by LC-MS to determine the total drug in solution. This amount (in moles) was divided by the estimated volume of each tumor to determine the approximate intratumoral concentration.

### Chemical Activation and Cellular Immunostimulation

Bone marrow-derived macrophages (BMDM) were generated from IL12-eYFP reporter mice by dissecting the femurs, flushing out the bone marrow, and culturing isolate with M-CSF supplemented growth media (Luthria G, et al. Nat Commun. 2020; 11:3521]. BMDM were plated in a 96 well plate at a concentration of 5,000 cells per well. These wells were then treated with either free gardiquimod, pATFB-SIL-Mal-Gard (13), or prodrug 13 that had been treated with 10 μM tris(2-carboxyethyl) phosphine) to chemically release the free gardiquimod (all drugs at a concentration of 100 nM). The cells were then incubated for 48 hours then fixed with 4% PFA and stained with Phospho-NF-κB p65 (Ser536) (93H1) Rabbit mAb and AF647 Goat anti-Rabbit IgG. Images were captured on a modified BX63 (Olympus) inverted microscopy system equipped with an environmental chamber and robotic stage, using excitation/emission wavelengths of 513/527 and 640/670 for eYFP and Cy5, respectively. The images were then quantified using ImageJ software (NIH, USA) to determine relative protein expression.

For radiation release experiments, 10 μM solutions of the albumin-gardiquimod conjugate were prepared in PBS (pH 7.4). X-ray irradiation was performed using a Precision (Madison, CT, USA) X-Rad320 at a rate of 330±10 cGy/min until the desired dosage was achieved. Irradiated samples were incubated at room temperature for 1 hour following irradiation to allow for the drug to fully release. All samples were then analyzed via LC-MS and compared to a previously prepared calibration curve (5 point curve utilizing the M+H+ ESI signal) to determine the amount of free drug generated (results shown in FIG. 19)

A number of embodiments of the present disclosure have been described. Nevertheless, it will be understood that various modifications may be made without departing from the spirit and scope of the present disclosure.

1. A compound of Formula (I), or a pharmaceutically acceptable salt thereof,
[RSM]-Linker-Drug Moiety (I)
wherein RSM is a radiation-sensitive moiety.

2. The compound of Formula (I), wherein the Linker comprises a carbamate group and an optional Solubility Modifier.

3. A compound of Formula (I-A), or a pharmaceutically acceptable salt thereof, 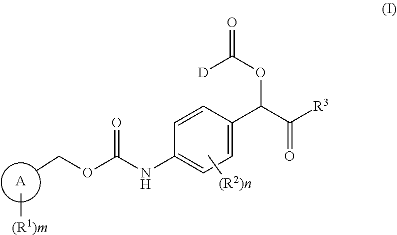
wherein:
Ring A is a 5-6 membered heteroaryl or a phenyl;
each R¹ is independently halogen, azido, or C1-C6 alkoxy, wherein not more than one of R¹ is azido;
m is 2, 3, 4, or 5;
each R² is independently halogen, C1-C6 alkyl, or C1-C6 haloalkyl;
n is 0, 1, 2, 3, or 4;
R³ is —OH, —O(C1-C6 alkyl), —NHR^{A}, or —NHR^{B};
R^{A} is hydrogen or C1-C6 alkyl;
R^{B} is 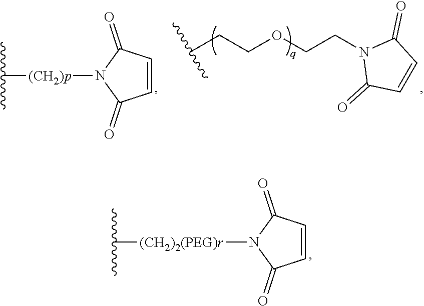
—(CH2)s-X, or -(PEG)t-X;
X is an electrophilic group;
p, q, r, s, and t are each an independently selected integer from 2-20; and
D is a Drug Moiety.

4. The compound of claim 1, wherein Ring A is phenyl.

5. The compound of claim 1, wherein m is 2 or 5.

6. The compound of claim 1, wherein m is 2 and each R¹ is methoxy.

7. The compound of claim 1, wherein m is 5, one R¹ is azido, and the remaining R¹ are each fluoro.

8. The compound of claim 1, wherein n is 0.

9. The compound of claim 1, wherein R³ is —NHR^{B}.

10. The compound of claim 1, wherein R^{B} is R^{B} is

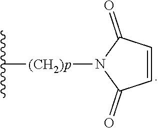

11. The compound of claim 1, wherein R^{B} is R^{B} is

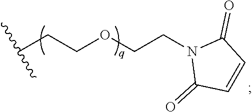

12. The compound of claim 1, wherein R^{B} is R^{B} is

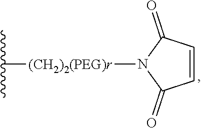

13. The compound of claim 1, wherein R³ is —OH.

14. The compound of claim 1, wherein R³ is —O(C1-C6 alkyl).

15. The compound of claim 1, wherein R³ is —NHR^{A}.

16. The compound of claim 1, wherein R^{A} is hydrogen.

17. The compound of claim 1, wherein R^{A} is C1-C6 alkyl.

18. The compound of claim 1, wherein D is a cytotoxic, cytostatic or immunomodulatory agent.

19. The compound of claim 1, wherein D is selected from antitubulin agents, DNA replication inhibitors, alkylating agents, antifolates, antimetabolites, chemotherapy sensitizers, topoisomerase inhibitors, and vinca alkaloids.

20. The compound of claim 1, wherein D is selected from MMAE, doxorubicin, and gardiquimod.

21. A pharmaceutical composition comprising a compound of claim 1, or a pharmaceutically acceptable salt thereof, and one or more pharmaceutically acceptable excipients.

22. A method of treating a disease or disorder in a subject in need thereof, comprising administering to the subject a therapeutically effective amount of a compound of claim 1, or the pharmaceutical composition of claim 21, and administering to the subject an effective amount of radiation.

23. The method of claim 22, wherein the disease or disorder is cancer.

24. The method of claim 22, wherein the effective amount of radiation is a therapeutically effective amount of radiation.

25. The method of claim 22, wherein the radiation is ionizing radiation.
